(19) United States
(12) Patent Application Publication
(10) Pub. No.: US 2020/0000831 A1
(43) Pub. Date: Jan. 2, 2020

(54) ANIMAL THERAPEUTIC AND FEED COMPOSITIONS AND METHODS OF USE

(71) Applicant: Cadena Bio, Inc., Cambridge, MA (US)

(72) Inventors: John M. GEREMIA, Cambridge, MA (US); Anastasia V. MURPHY, Cambridge, MA (US)

(21) Appl. No.: 16/293,140

(22) Filed: Mar. 5, 2019

Related U.S. Application Data
(63) Continuation of application No. 15/775,501, now abandoned.
(60) Provisional application No. 62/255,348, filed on Nov. 13, 2015.
(60) Provisional application No. 62/255,352, filed on Nov. 13, 2015.

(51) Int. Cl. A61K 31/702; A23K 20/163; A23K 50/75; A23K 50/30; A61P 1/00; A61K 9/00

(52) U.S. Cl. CPC A61K 31/702; A23K 20/163; A23K 50/75; A61K 9/0056; A61P 1/00; A61K 9/0095; A23K 50/30

(57) ABSTRACT

Provided herein are oligosaccharide compositions for administration to animals suitable for improving animal health, including, for example, to treat diseases or disorders or to enhance animal growth.

### FIELD

The present disclosure relates generally to therapeutic compositions that are made up of oligosaccharide compositions, and methods of formulating and using such compositions (e.g., as animal feed compositions or animal feed pre-mixes) to improve animal health.

### BACKGROUND

Many factors can affect the growth and activity of gastrointestinal microorganisms in an animal, and which in turn can affect animal health. For example, certain carbohydrates can undergo selective fermentation by beneficial bacteria, increasing their presence in the gut relative to other bacteria. The use of carbohydrates presents various opportunities for animal health, particularly for treating or preventing metabolic, immune and infectious diseases in animals. Currently, diseases of the digestive system are typically treated with antibiotics or corticosteroids, which may often have undesired side effects. Thus, there is a need in the art for compositions that can be used to improve animal health.

### BRIEF SUMMARY

Provided herein are compositions suitable for use to improve animal health, including (i) enhancing growth in animals, (ii) reducing occurrence of a disease or disorder in animals, and/or (iii) treating a disease or disorder in animals. Thus, in some aspects, provided herein is a method of enhancing growth in an animal by administering a therapeutic composition to the animal. In other aspects, provided herein is a method of reducing occurrence of a disease or disorder in an animal by administering a therapeutic composition to the animal. In yet other aspects, provided herein is a method of treating a disease or disorder in an animal by administering a therapeutic composition to the animal.

The compositions administered to the animals are formulated to target certain regions of the gastrointestinal tract in the animals and/or modulate at least a portion of the gut microbiome in the animals to improve animal health. Thus, in some aspects, provided herein is a method of targeting a region of the gastrointestinal tract in an animal by administering a therapeutic composition disclosed herein to the animal. In some variations, the therapeutic composition targets the ileum and/or cecum in the gastrointestinal tract in the animal. In other aspects, provided is a method of modulating the gut microbiome of an animal by administering a therapeutic composition to the animal.

In some embodiments of any of the methods disclosed herein, the therapeutic composition comprises an oligosaccharide composition, and optionally at least one pharmaceutically acceptable vehicle. In some variations, the oligosaccharide composition has a glycosidic bond type distribution of at least 10 mol % α-(1,3) glycosidic linkages, and at least 10 mol % β-(1,3) glycosidic linkages; and wherein at least 10 dry wt % of the oligosaccharide composition has a degree of polymerization of at least 3. In other variations, the oligosaccharide composition has a glycosidic bond type distribution of less than 9 mol % α-(1,4) glycosidic linkages and less than 19 mol % α-(1,6) glycosidic linkages; and wherein at least 10 dry wt % of the oligosaccharide composition has a degree of polymerization of at least 3. In yet other variations, the oligosaccharide composition has a glycosidic bond type distribution of at least 15 mol % β-(1,2) glycosidic linkages. In yet other variations, at least 50 dry wt % of the oligosaccharide composition has a degree of polymerization of at least 3. In one variation, the oligosaccharide composition is produced according to the methods described herein.

In some embodiments of any of the methods disclosed herein, the therapeutic composition comprises:
(a) at least one C5 carbohydrate, at least one C6 carbohydrate, at least one C5 deoxy sugar, at least one C6 deoxy sugar, at least one C5 amino sugar, at least one C6 amino sugar, at least one C5 sugar alcohol, at least one C6 sugar alcohol, at least one C5 sugar acid, at least one C6 sugar acid, at least one C5 phosphate sugar, at least one C6 phosphate sugar, at least one C5 sulfate sugar, or at least one C6 sulfate sugar, or
   a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or
   any combinations thereof; and
(b) a delivery vehicle.

In other aspects, provided is a method of producing a therapeutic composition, by: combining feed sugar with a catalyst to form a reaction mixture; and producing an oligosaccharide composition from at least a portion of the reaction mixture; and optionally combining the oligosaccharide composition with a pharmaceutically acceptable vehicle. In embodiments of the foregoing, the catalyst is a polymeric catalyst that includes acidic monomers and ionic monomers connected to form a polymeric backbone; or the catalyst is a solid-supported catalyst that includes a solid support, acidic moieties attached to the solid support, and ionic moieties attached to the solid support.

The therapeutic composition described above may be incorporated into an animal's diet to improve health of the animal. Thus, in some variations, provided is an animal feed composition comprising:
(a) a base feed; and
(b) any of the therapeutic compositions described herein.

In other variations, provided is an animal feed pre-mix comprising:
(a) a carrier material; and
(b) any of the therapeutic compositions described herein.

Provided herein are also the use of such therapeutic compositions, animal fees compositions, and animal feed pre-mixes to improve animal health, including treating diseases and disorders in animals.

### DESCRIPTION OF THE FIGURES

The present application can be understood by reference to the following description taken in conjunction with the accompanying figures.

FIG. 1 depicts an exemplary process to produce an oligosaccharide composition from sugars in the presence of a catalyst.

FIG. 2A illustrates a portion of a catalyst with a polymeric backbone and side chains.

FIG. 2B illustrates a portion of an exemplary catalyst, in which a side chain with the acidic group is connected to the polymeric backbone by a linker and in which a side chain with the cationic group is connected directly to the polymeric backbone.

FIG. 3 depicts a reaction scheme to prepare a dual-functionalized catalyst from an activated carbon support, in which the catalyst has both acidic and ionic moieties.

FIG. 4 illustrates a portion of a polymeric catalyst, in which the monomers are arranged in blocks of monomers, and the block of acidic monomers alternates with the block of ionic monomers.

FIG. 5A illustrates a portion of a polymeric catalyst with cross-linking within a given polymeric chain.

FIG. 5B illustrates a portion of a polymeric catalyst with cross-linking within a given polymeric chain.

FIG. 6A illustrates a portion of a polymeric catalyst with cross-linking between two polymeric chains.

FIG. 6B illustrates a portion of a polymeric catalyst with cross-linking between two polymeric chains.

FIG. 6C illustrates a portion of a polymeric catalyst with cross-linking between two polymeric chains.

FIG. 6D illustrates a portion of a polymeric catalyst with cross-linking between two polymeric chains.

FIG. 7 illustrates a portion of a polymeric catalyst with a polyethylene backbone.

FIG. 8 illustrates a portion of a polymeric catalyst with a polyvinylalcohol backbone.

FIG. 9 illustrates a portion of a polymeric catalyst, in which the monomers are randomly arranged in an alternating sequence.

FIG. 10 illustrates two side chains in a polymeric catalyst, in which there are three carbon atoms between the side chain with the Bronsted-Lowry acid and the side chain with the cationic group.

FIG. 11 illustrates two side chains in a polymeric catalyst, in which there are zero carbons between the side chain with the Bronsted-Lowry acid and the side chain with the cationic group.

FIG. 12 illustrates a portion of a polymeric catalyst with an ionomeric backbone.

FIG. 13 depicts an exemplary process to produce a functionalized oligosaccharide composition, wherein a portion of an oligosaccharide comprising pendant functional groups and bridging functional groups is shown.

FIG. 14 is a diagram showing selective growth of various gut microflora on carbohydrate sources.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

Provided herein are compositions (e.g., therapeutic compositions, animal feed compositions, and animal feed pre-mixes) suitable for administration to animals. The administration of such therapeutic compositions to animals can improve the overall health of the animals. For example, in some aspects, the administration of such therapeutic compositions to animals can reduce the occurrence of a disease or disorder in animals. In other aspects, the administration of such therapeutic compositions to animals can treat a disease or disorder in animals.

Types of Animals

Various types of animals may be administered the therapeutic compositions described herein. In some embodiments, the animal is poultry, such as a chicken, a duck, a turkey, a goose, a quail, or a Cornish game hen. In some embodiments, the poultry is a layer hen, a broiler chicken, or a turkey. In some embodiments, the animal is a mammal, such as a cow, a pig, a goat, a sheep, a deer, a bison, a rabbit, an alpaca, a llama, a mule, a horse, a reindeer, a water buffalo, a yak, a guinea pig, a rat, a mouse, an alpaca, a dog, or a cat. In some embodiments, the animal is an aquatic animal, such as a trout, a salmon, a bass, a tilapia, a shrimp, an oyster, a mussel, a clam, a lobster, or a crayfish. In some embodiments, the animal is monogastric (i.e., having a single-chambered stomach). In some embodiments, the animal is a ruminant (i.e., having a multi-chambered stomach). In some embodiments, the animal is a ruminant in the pre-ruminant phase, such as nursery calves.

In some variations, the animal is other than a human (or is a non-human animal). In other variations, the animal is other than a laboratory animal, e.g., whose primary use is for research and testing purposes.

The animals administered the therapeutic compositions described herein may include livestock, as well as companion animals and pets. For example, in one variation, the compositions described herein may be formulated as an animal feed composition suitable for feeding to livestock. In other variations, the therapeutic compositions described herein may be formulated as a medicament suitable for administering to a pet to treat certain diseases or disorders. In yet other variations, the compositions described herein may be formulated for us in aquaculture.

The compositions described herein may be administered to a single animal, or to an animal population or a subset thereof.

The therapeutic compositions comprise an oligosaccharide composition, and optionally at least one pharmaceutically acceptable vehicle and optionally other compounds and agents.

In some variations, the therapeutic compositions described herein target specific regions of the gastrointestinal tract in the animals and/or modulate at least a portion of the gut microbiome in the animals to improve animal health. Thus, in some aspects, provided is a therapeutic composition comprising any of the oligosaccharide compositions described herein; and optionally at least one pharmaceutically acceptable vehicle. In certain variations, the therapeutic compositions target specific regions of the gastrointestinal tract in the animals where digestibility of the oligosaccharide compositions are maximized. For example, such specific regions of the gastrointestinal tract in animals include the ileum and/or cecum.

The therapeutic compositions and their uses are described herein further detail below.

### Compositions

Oligosaccharide Compositions

The compositions (e.g., therapeutic compositions, animal feed compositions, or animal feed pre-mixes) comprise oligosaccharide compositions, and are suitable for non-human consumption. The oligosaccharide compositions produced according to the methods described herein and the properties of such compositions may vary, depending on the type of sugars as well as the reaction conditions used. The oligosaccharide compositions may be characterized based on the type of oligosaccharides present, degree of polymerization, glass transition temperature, and hygroscopicity.

Types of Oligosaccharides

In some embodiments, the oligosaccharide compositions include an oligosaccharide comprising one type of sugar monomer. For example, in some embodiments, the oligosaccharide compositions may include a gluco-oligosaccharide, a galacto-oligosaccharide, a fructo-oligosaccharide, a manno-oligosaccharide, an arabino-oligosaccharide, or a xylo-oligosaccharide, or any combinations thereof. In some embodiments, the oligosaccharide compositions include an oligosaccharide comprising two different types of sugar monomers. For example, in some embodiments, the oligosaccharide compositions may include a gluco-galacto-oligosaccharide, a gluco-fructo-oligosaccharide, a gluco-manno-oligosaccharide, a gluco-arabino-oligosaccharide, a gluco-xylo-oligosaccharide, a galacto-fructo-oligosaccharide, a galacto-manno-oligosaccharide, a galacto-arabino-oligosaccharide, a galacto-xylo-oligosaccharide, a fructo-manno-oligosaccharide, a fructo-arabino-oligosaccharide, a fructo-xylo-oligosaccharide, a manno-arabino-oligosaccharide, a manno-xylo-oligosaccharide, or an arabino-xylo-oligosaccharide, or any combinations thereof. In some embodiments, the oligosaccharide compositions include an oligosaccharide comprising more than two different types of sugar monomers. In some variations, the oligosaccharide compositions include an oligosaccharide comprising 3, 4, 5, 6, 7, 8, 9, or 10 different types of sugar monomers. For example, in certain variations the oligosaccharide compositions include an oligosaccharide comprising a galacto-arabino-xylo-oligosaccharide, a fructo-galacto-xylo-oligosaccharide, a arabino-fructo-manno-xylo-oligosaccharide, a gluco-fructo-galacto-arabino-oligosaccharide, a fructo-gluco-arabino-manno-xylo oligosaccharide, or a gluco-galacto-fructo-manno-arabino-xylo-oligosaccharide.

As used herein, “oligosaccharide” refers to a compound containing two or more monosaccharide units linked by glycosidic bonds.

As used herein, “gluco-oligosaccharide” refers to a compound containing two or more glucose monosaccharide units linked by glycosidic bonds. Similarly, “galacto-oligosaccharide” refers to a compound containing two or more galactose monosaccharide units linked by glycosidic bonds.

As used herein, “gluco-galacto-oligosaccharide” refers to a compound containing one or more glucose monosaccharide units linked by glycosidic bonds, and one or more galactose monosaccharide units linked by glycosidic bonds. In some embodiments, the ratio of glucose to galactose on a dry mass basis is between 10:1 glucose to galactose to 0.1:1 glucose to galactose, 5:1 glucose to galactose to 0.2:1 glucose to galactose, 2:1 glucose to galactose to 0.5:1 glucose to galactose. In one embodiment, the ratio of glucose to galactose is 1:1.

In one variation, the oligosaccharide composition is a long oligosaccharide composition, while in another variation the oligosaccharide composition is a short oligosaccharide composition. As used herein, the term “long oligosaccharide composition” refers to an oligosaccharide composition with an average degree of polymerization (DP) of about 8, about 9, about 10, about 11, about 12, about 13, about 14, about 15, about 16, about 17, about 18, about 19, or about 20. As used herein, the term “short oligosaccharide composition” refers to oligosaccharide composition with an average DP of about 2, about 3, about 4, about 5, about 6, or about 7.

In some variations, the compositions described herein comprise:

at least one carbohydrate, or

at least one deoxy sugar, or

at least one amino sugar, or

at least one sugar alcohol, or

at least one sugar acid, or

at least one phosphate sugar, or

at least one sulfate sugar, or

a compound comprising 2 to 5 units, wherein each unit is independently a carbohydrate unit, a deoxy sugar unit, an amino sugar unit, a sugar alcohol unit, a sugar acid unit, a phosphate sugar unit, or a sulfate sugar unit, or

any combinations of the foregoing.

In some embodiments, a carbohydrate is a molecule that consists of carbon, hydrogen and oxygen atoms. The empirical formula for carbohydrate may be expressed as Cₘ(H₂O)ₙ, where m and n are integers and may be different or the same.

In some embodiments, a deoxy sugar is a carbohydrate in which at least one —OH moiety has been replaced with a hydrogen. Examples of deoxy sugars include fucose and rhamnose.

In some embodiments, an amino sugar is a carbohydrate in which at least one —OH moiety has been replaced with an amine group. Examples of amino sugars include glucosamine and galactosamine.

In some embodiments, a sugar alcohol is a carbohydrate in which at least one —C═O moiety has been replaced with a —HC—OH. In other embodiments, a sugar alcohol is a compound having the formula HOCH₂(CHOH)ₚCH₂OH, wherein p is an integer. Examples of sugar alcohols include glucitol, sorbitol, xylitol, lactitol, arabinatol, arabitol, glycerol, erythritol, mannitol, galacitol, fucitol, iditol, inositol, and volemitol.

In some embodiments, a sugar acid is a carbohydrate in which at least one —COH or —HC═O moiety has been replaced with a —COOH. Examples of sugar acids include gluconic acid and glucuronic acid.

In some embodiments, a phosphate sugar is a carbohydrate in which at least one —OH moiety has been replaced with a phosphate group.

In some embodiments, a sulfate sugar is a carbohydrate in which at least one —OH moiety has been replaced with a sulfate group.

It should be understood that a compound may be described by one or more of the terms above. For example, a compound may be both a sugar acid and an amino sugar. One such example is N-acetylneuraminic acid.

In some variations of the foregoing, the carbohydrate, deoxy sugar, amino sugar, sugar alcohol, sugar acid, phosphate sugar, and sulfate sugar may be unsubstituted. In other variations, the carbohydrate, deoxy sugar, amino sugar, sugar alcohol, sugar acid, phosphate sugar, and sulfate sugar may substituted with one or more substituents. In one variation, the one or more substituents are independently selected from acyl, amino, hydroxyl, carboxylic acid, sulfur trioxide, sulfate, and phosphate. In another variation, the one or more substituents are independently selected from acyl, amino, alcohol, carboxylic acid, sulfate, phosphate, and sulfur oxide. In yet another variation, the one or more substituents are independently selected from acyl, amino, alcohol, carboxylic acid, sulfate and phosphate.

In one variation, the carboxylic acid substituent includes lactic acid, acetic acid, formic acid, citric acid, pyruvic acid, succinic acid, glutamic acid, aspartic acid, butyric acid, itaconic acid, malic acid, maleic acid, propionic acid, butanoic acid, pentanoic acid, hexanoic acid, adipic acid, isobutyric acid, formic acid, levulinic acid, valeric acid, or isovaleric acid.

In another variation, the alcohol substituent includes ethanol, propanol, butanol, pentanol, hexanol, propanediol, butanediol, and pentanediol.

For example, in one variation, the composition comprises neuraminic acid. Neuraminic acid is a compound that can be described as an amino sugar, a sugar acid, or a deoxy sugar that is substituted with an alcohol, which in this instance is a polyol. In other examples, in other variations, the composition comprises a sialic acid. Sialic acids are a class of compounds that are N- or O-substituted derivatives of neuraminic acid.

In other embodiments, the composition comprises a compound comprising 2 to 5 units, wherein each unit is independently a carbohydrate unit, a deoxy sugar unit, an amino sugar unit, a sugar alcohol unit, a sugar acid unit, a phosphate sugar unit, or a sulfate sugar unit.

It should be generally understood that the 2 to 5 units of the compound are connected together by at least one bond. The units may be the same or different. In some variations, 2 to 5 units of the compound are connected together by at least one glycosidic bond. The glycosidic bonds may be the same type of glycosidic bond or different types of glycosidic bonds. Examples of glycosidic bond types includes α-1,4 bonds, α-1,2 bonds, β-1,2 bonds, α-1,3 bonds, β-1,3 bonds, β-1,4 bonds, α-1,6 bonds, and α-1,6 bonds.

For example, in some embodiments, the compound comprises 2 units. In some variations, both units are the same type of unit. For example, in one variation, both units are carbohydrate units. In other variations, both units are different types of unit. For example, in one variation, one unit is a deoxy sugar unit and the other unit is an amino sugar unit.

In other embodiments, the compound comprises 3, 4, or 5 units. In some variations, one or more of the units are the same types of units. In other variations, one or more of the units are different types of units. In yet other variations, the 3, 4, or 5 units are connected by the same or different types of bonds. For example, in certain variations, the compound comprises 3 units, which are connected together by glycosidic bonds. In one variation of such compound, the glycosidic bonds are the same type of glycosidic bond. In other variations of such compound, at least one of the glycosidic bonds are different types of glycosidic bonds.

In certain variations, the compositions described herein comprise:

at least one C5 carbohydrate, or

at least one C6 carbohydrate, or

at least one C5 deoxy sugar, or

at least one C6 deoxy sugar, or

at least one C5 amino sugar, or

at least one C6 amino sugar, or

at least one C5 sugar alcohol, or

at least one C6 sugar alcohol, or

at least one C5 sugar acid, or

at least one C6 sugar acid, or

at least one C5 phosphate sugar, or

at least one C6 phosphate sugar, or

at least one C5 sulfate sugar, or

at least one C6 sulfate sugar, or

a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or

any combinations of the foregoing.

In some embodiments, a C5 carbohydrate is a molecule that consists of five carbon atoms, as well as hydrogen and oxygen atoms. The empirical formula for a C5 carbohydrate may be expressed as C₅(H₂O)ₙ, wherein n is an integer. Examples of C5 carbohydrates include ribose, xylose, and arabinose.

In some embodiments, a C6 carbohydrate is a molecule that consists of six carbon atoms, as well as hydrogen and oxygen atoms. The empirical formula for a C6 carbohydrate may be expressed as C₆(H₂O)ₙ, wherein n is an integer. Examples of C6 carbohydrates include allose, fructose, glucose, mannose, and galactose.

It should be understood that a C5 deoxy sugar or a C6 deoxy sugar is a C5 carbohydrate or a C6 carbohydrate, respectively, in which at least one —OH moiety has been replaced with a hydrogen. Similarly, a C5 amino sugar or a C6 amino sugar is a C5 carbohydrate or C6 carbohydrate in which at least one —OH moiety has been replaced with an amine group; a C5 sugar alcohol or a C6 sugar alcohol is a C5 carbohydrate or C6 carbohydrate in which at least one —C═O moiety has been replaced with a —HC—OH; a C5 sugar acid or a C6 sugar acid is a C5 carbohydrate or C6 carbohydrate in which at least one —COH or —HC═O moiety has been replaced with a —COOH; a C5 phosphate sugar or a C6 phosphate sugar is a C5 carbohydrate or C6 carbohydrate in which at least one —OH moiety has been replaced with a phosphate group; and a C5 sulfate sugar or C6 sulfate sugar is a C5 carbohydrate or C6 carbohydrate in which at least one —OH moiety has been replaced with a sulfate group.

In certain variations, the compositions comprise cellobiose, isomaltulose, lactose, maltose, melibiose, sucrose, acarviosin, n-acetyllactosamine, allolactose, chitobiose, glactose-alpha-1,3-galactose, gentiobiose, isomalt, isomaltulose, kojibiose, lactitol, lactobionic acid, lactulose, laminaribiose, maltitol, mannobiose, melibiulose, neohesperidose, nigerose, robinose, rutinose, sambubuise, sophorose, sucralfate, sucralose, sucrose acetate isobutyrate, sucrose octaacetate, trehalose, turanose, vicianose, xylobiose, isomaltotriose, nigerotriose, maltotriose, melezitose, maltotriulose, raffinose, kestose, lychnose, maltotetraose, nigerotetraose, nystose, sesamose, or stachyose, or any combinations thereof.

The compositions may comprise any combinations of the carbohydrates and sugars described above.

Functionalized Oligosaccharide Compositions

In some variations, the oligosaccharide compositions described herein are functionalized oligosaccharide compositions. Functionalized oligosaccharide compositions may be produced by, for example, combining one or more sugars (e.g., feed sugars) with one or more functionalizing compounds in the presence of a catalyst, including, for example, polymeric catalysts and solid-supported catalysts as described in WO 2012/118767 and WO 2014/031956. In certain variations, a functionalized oligosaccharide is a compound comprising two or more monosaccharide units linked by glycosidic bonds in which one or more hydroxyl groups in the monosaccharide units are independently replaced by a functionalizing compound, or comprise a linkage to a functionalizing compound. The functionalizing compound may be a compound that can attach to the oligosaccharide through an ether, ester, oxygen-sulfur, amine, or oxygen-phosphorous bond, and which does not contain a monosaccharide unit.

Functionalizing Compounds

In certain variations, the functionalizing compound comprises one or more functional groups independently selected from amine, hydroxyl, carboxylic acid, sulfur trioxide, sulfate, and phosphate. In some variations, one or more functionalizing compounds are independently selected from the group consisting of amines, alcohols, carboxylic acids, sulfates, phosphates, or sulfur oxides.

In some variations, the functionalizing compound has one or more hydroxyl groups. In some variations, the functionalizing compound with one or more hydroxyl groups is an alcohol. Such alcohols may include, for example, alkanols and sugar alcohols.

In certain variations, the functionalizing compound is an alkanol with one hydroxyl group. For example, in some variations, the functionalizing compound is selected from ethanol, propanol, butanol, pentanol, and hexanol. In other variations, the functionalizing compound has two or more hydroxyl groups. For example, in some variations, the functionalizing compound is selected from propanediol, butanediol, and pentanediol.

For example, in one variation, one or more sugars (e.g., feed sugars) may be combined with a sugar alcohol in the presence of a polymeric catalyst to produce a functionalized oligosaccharide composition. Suitable sugar alcohols may include, for example, sorbitol (also known as glucitol), xylitol, lacitol, arabinatol (also known as arabitol), glycerol, erythritol, mannitol, galacitol, fucitol, iditol, inositol, or volemitol, or any combinations thereof.

In another variation, wherein the functionalizing compound comprises a hydroxyl group, the functionalizing compound may become attached to the monosaccharide unit through an ether bond. The oxygen of the ether bond may be derived from the monosaccharide unit, or from the functionalizing compound.

In yet other variations, the functionalizing compound comprises one or more carboxylic acid functional groups. For example, in some variations, the functionalizing compound is selected from lactic acid, acetic acid, citric acid, pyruvic acid, succinic acid, glutamic acid, itaconic acid, malic acid, maleic acid, propionic acid, butanoic acid, pentanoic acid, hexanoic acid, adipic acid, isobutyric acid, formic acid, levulinic acid, valeric acid, and isovaleric acid. In other variations, the functionalizing compound is a sugar acid. For example, in one embodiment, the functionalizing compound is gluconic acid. In certain variations, wherein the functionalizing compound comprises a carboxylic acid group, the functionalizing compound may become attached to the monosaccharide unit through an ester bond. The non-carbonyl oxygen of the ester bond may be derived from the monosaccharide unit, or from the functionalizing compound.

In still other variations, the functionalizing compound comprises one or more amine groups. For example, in some variations, the functionalizing compound is an amino acid, while in other variations the functionalizing compound is an amino sugar. In one variation, the functionalizing compound is selected from glutamic acid, aspartic acid, glucosamine and galactosamine. In certain variations, wherein the functionalizing compound comprises an amine group, the functionalizing compound may become attached to the monosaccharide unit through an amine bond.

In yet other variations, the functionalizing compound comprises a sulfur trioxide group or a sulfate group. For example, in one variation, the functionalizing compound is dimethylformamide sulfur trioxide complex. In another variation, the functionalizing compound is sulfate. In one embodiment, the sulfate is produced in situ, from, for example, sulfur trioxide. In certain variations wherein the functionalizing compound comprises a sulfur trioxide or sulfate group, the functionalizing compound may become attached to the monosaccharide unit through an oxygen-sulfur bond.

In still other variations, the functionalizing compound comprises a phosphate group. In certain variations wherein the functionalizing compound comprises a phosphate group, the functionalizing compound may become attached to the monosaccharide unit through an oxygen-phosphorous bond.

It should be understood that the functionalizing compounds described herein may contain a combination of functional groups. For example, the functionalizing compound may comprise one or more hydroxyl groups and one or more amine groups (for example, amino sugars). In other embodiments, the functionalizing compound may comprise one or more hydroxyl groups and one or more carboxylic acid groups (for example, sugar acids). In yet other embodiments, the functionalizing compound may comprise one or more amine groups and one or more carboxylic acid groups (for example, amino acids). In still other embodiments, the functionalizing compound comprises one or more additional functional groups, such as esters, amides, and/or ethers. For example, in certain embodiments, the functionalizing compound is a sialic acid (for example, N-acetylneuraminic acid, 2-keto-3-deoxynonic acid, and other N- or O-substituted derivatives of neuraminic acid).

It should further be understood that a functionalizing compound may belong to one or more of the groups described above. For example, a glutamic acid is both an amine and a carboxylic acid, and a gluconic acid is both a carboxylic acid and an alcohol.

In some variations, the functionalizing compound forms a pendant group on the oligosaccharide. In other variations, the functionalizing compound forms a bridging group between an oligomer backbone and a second oligomer backbone; wherein each oligomer backbone independently comprises two or more monosaccharide units linked by glycosidic bonds; and the functionalizing compound is attached to both backbones. In other variations, the functionalizing compound forms a bridging group between an oligomer backbone and a monosaccharide; wherein the oligomer backbone comprises two or more monosaccharide units linked by glycosidic bonds; and the functionalizing compound is attached to the backbone and the monosaccharide.

Pendant Functional Groups

In certain variations, combining one or more sugars (e.g., feed sugars) and one or more functionalizing compounds in the presence of a catalyst, including polymeric catalysts and solid-supported catalysts as described in WO 2012/118767 and WO 2014/031956, produces a functionalized oligosaccharide composition. In certain embodiments, a functionalizing compound is attached to a monosaccharide subunit as a pendant functional group.

A pendant functional group may include a functionalization compound attached to one monosaccharide unit, and not attached to any other monosaccharide units. In some variations, the pendant functional group is a single functionalization compound attached to one monosaccharide unit. For example, in one variation, the functionalizing compound is acetic acid, and the pendant functional group is acetate bonded to a monosaccharide through an ester linkage. In another variation, the functionalizing compound in propionic acid, and the pendant functional group is propionate bonded to a monosaccharide through an ester linkage. In yet another variation, the functionalizing compound is butanoic acid, and the pendant functional group is butanoate bonded to a monosaccharide through an ester linkage. In other variations, a pendant functional group is formed from linking multiple functionalization compounds together. For example, in some embodiments, the functionalization compound is glutamic acid, and the pendant functional group is a peptide chain of two, three, four, five, six, seven, or eight glutamic acid residues, wherein the chain is attached to a monosaccharide through an ester linkage. In other embodiments, the peptide chain is attached to the monosaccharide through an amine linkage.

The pendant functional group may comprise a single linkage to the monosaccharide, or multiple linkages to the monosaccharide. For example, in one embodiment, the functionalization compound is ethanediol, and the pendant functional group is ethyl connected to a monosaccharide through two ether linkages.

Referring to FIG. 13, process 1300 depicts an exemplary scheme to produce an oligosaccharide containing different pendant functional groups. In process 1300, monosaccharides 1302 (represented symbolically) are combined with the functionalizing compound ethane diol 1304 in the presence of catalyst 1306 to produce an oligosaccharide. Portion 1310 of the oligosaccharide is shown in FIG. 13, wherein the monosaccharides linked through glycosidic bonds are represented symbolically by circles and lines. The oligosaccharide comprises three different pendant functional groups, as indicated by the labeled section. These pendant functional groups include a single functionalization compound attached to a single monosaccharide unit through one linkage; two functionalization compounds linked together to form a pendant functional group, wherein the pendant functional group is linked to a single monosaccharide unit through one linkage; and a single functionalization compound attached to a single monosaccharide unit through two linkages. It should be understood that while the functionalization compound used in process 1300 is ethanediol, any of the functionalization compounds or combinations thereof described herein may be used. It should be further understood that while a plurality of pendant functional groups is present in portion 1310 of the oligosaccharide, the number and type of pendant functional groups may vary in other variations of process 1300.

It should be understood that any functionalization compounds may form a pendant functional group. In some variations, the functionalized oligosaccharide composition contains one or more pendant groups selected from the group consisting of glucosamine, galactosamine, citric acid, succinic acid, glutamic acid, aspartic acid, glucuronic acid, butyric acid, itaconic acid, malic acid, maleic acid, propionic acid, butanoic acid, pentanoic acid, hexanoic acid, adipic acid, isobutyric acid, formic acid, levulinic acid, valeric acid, isovaleric acid, sorbitol, xylitol, arabitol, glycerol, erythritol, mannitol, galacitol, fucitol, iditol, inositol, volemitol, lacitol, ethanol, propanol, butanol, pentanol, hexanol, propanediol, butanediol, pentanediol, sulfate and phosphate.

Bridging Functional Groups

In certain variations, combining one or more sugars (e.g., feed sugars) and one or more functionalizing compounds in the presence of a catalyst, including polymeric catalysts and solid-supported catalysts as described in WO 2012/118767 and WO 2014/031956, produces a functionalized oligosaccharide comprising a bridging functional group.

Bridging functional groups may include a functionalization compound attached to one monosaccharide unit and attached to at least one additional monosaccharide unit. The monosaccharide units may independently be monosaccharide units of the same oligosaccharide backbone, monosaccharide units of separate oligosaccharide backbones, or monosaccharide sugars that are not bonded to any additional monosaccharides. In some variations, the bridging functional compound is attached to one additional monosaccharide unit. In other variations, the bridging functional compound is attached to two or more additional monosaccharide units. For example, in some embodiments, the bridging functional compound is attached to two, three, four, five, six, seven, or eight additional monosaccharide units. In some variations, the bridging functional group is formed by linking a single functionalization compound to two monosaccharide units. For example, in one embodiment, the functionalization compound is glutamic acid, and the bridging functional group is a glutamate residue attached to one monosaccharide unit through an ester bond, and an additional monosaccharide unit through an amine bond. In other embodiments, the bridging functionalization group is formed by linking multiple functionalization compound molecules to each other. For example, in one embodiment, the functionalization compound is ethanediol, and the bridging functional group is a linear oligomer of four ethanediol molecules attached to each other through ether bonds, the first ethanediol molecule in the oligomer is attached to one monosaccharide unit through an ether bond, and the fourth ethanediol molecule in the oligomer is attached to an additional monosaccharide unit through an ether bond.

Referring again to FIG. 13, portion 1310 of the oligosaccharide produced according to process 100 comprises three different bridging functional groups, as indicated by the labeled section. These bridging functional groups include a single functionalization compound attached to a monosaccharide unit of an oligosaccharide through one linkage, and attached to a monosaccharide sugar through an additional linkage; a single functionalization compound attached to two different monosaccharide units of the same oligosaccharide backbone; and two functionalization compounds linked together to form a bridging functional group, wherein the bridging functional group is linked to one monosaccharide unit through one linkage and to an additional monosaccharide unit through a second linkage. It should be understood that while the functionalization compound used in process 1300 is ethanediol, any of the functionalization compounds or combinations thereof described herein may be used. It should be further understood that while a plurality of bridging functional groups is present in portion 110 of the oligosaccharide, the number and type of bridging functional groups may vary in other variations of process 1300.

It should be understood that any functionalization compounds with two or more functional groups able to form bonds with a monosaccharide may form a bridging functional group. For example, bridging functional groups may be selected from polycarboxylic acids (such as succinic acid, itaconic acid, malic acid, maleic acid, and adipic acid), polyols (such as sorbitol, xylitol, arabitol, glycerol, erythritol, mannitol, galacitol, fucitol, iditol, inositol, volemitol, and lacitol), and amino acids (such as glutamic acid). In some variations, the functionalized oligosaccharide composition comprises one or more bridging groups selected from the group consisting of glucosamine, galactosamine, lactic acid, acetic acid, citric acid, pyruvic acid, succinic acid, glutamic acid, aspartic acid, glucuronic acid, itaconic acid, malic acid, maleic acid, adipic acid, sorbitol, xylitol, arabitol, glycerol, erythritol, mannitol, galacitol, fucitol, iditol, inositol, volemitol, lacitol, propanediol, butanediol, pentanediol, sulfate and phosphate.

Functionalized oligosaccharide compositions comprising a mixture of pendant functional groups and bridging functional groups may also be produced using the methods described herein. For example, in certain embodiments, one or more sugars are combined with a polyol in the presence of a catalyst, and a functionalized oligosaccharide composition is produced wherein at least a portion of the composition comprises pendant polyol functional groups attached to oligosaccharides through ether linkages, and at least a portion comprises bridging polyol functional groups wherein each group is attached to a first oligosaccharide through a first ether linkage and a second oligosaccharide through a second ether linkage.

It should further be understood that the one or more functionalization compounds combined with the sugars, oligosaccharide composition, or combination thereof may form bonds with other functionalization compounds, such that the functionalized oligosaccharide composition comprises monosaccharide units bonded to a first functionalization compound, wherein the first functionalization compound is bonded to a second functionalization compound.

Degree of Polymerization

The oligosaccharide content of reaction products can be determined, e.g., by a combination of high performance liquid chromatography (HPLC) and spectrophotometric methods. For example, the average degree of polymerization (DP) for the oligosaccharides can be determined as the number average of species containing one, two, three, four, five, six, seven, eight, nine, ten to fifteen, and greater than fifteen, anhydrosugar monomer units.

In some embodiments, the oligosaccharide degree of polymerization (DP) distribution for the one or more oligosaccharides after combining the one or more sugars with the catalyst (e.g., at 2, 3, 4, 8, 12, 24, or 48 hours after combining the one or more sugars with the catalyst) is: DP2=0%-40%, such as less than 40%, less than 30%, less than 20%, less than 10%, less than 5%, or less than 2%; or 10%-30% or 15%-25%; DP3=0%-20%, such as less than 15%, less than 10%, less than 5%; or 5%-15%; and DP4+=greater than 15%, greater than 20%, greater than 30%, greater than 40%, greater than 50%; or 15%-75%, 20%-40% or 25%-35%.

In some embodiments, the oligosaccharide degree of polymerization (DP) distribution for the one or more oligosaccharides after combining the one or more sugars with the catalyst (e.g., at 2, 3, 4, 8, 12, 24, or 48 hours after combining the one or more sugars with the catalyst) is any one of entries (1)-(192) of Table 1A.

TABLE 1A

| Entry | DP4+ (%) | DP3 (%) | DP2 (%) |
|---|---|---|---|
| 1 | 20-25 | 0-5 | 0-5 |
| 2 | 20-25 | 0-5 | 5-10 |
| 3 | 20-25 | 0-5 | 10-15 |
| 4 | 20-25 | 0-5 | 15-20 |
| 5 | 20-25 | 0-5 | 20-25 |
| 6 | 20-25 | 0-5 | 25-30 |
| 7 | 20-25 | 5-10 | 0-5 |
| 8 | 20-25 | 5-10 | 5-10 |
| 9 | 20-25 | 5-10 | 10-15 |
| 10 | 20-25 | 5-10 | 15-20 |
| 11 | 20-25 | 5-10 | 20-25 |
| 12 | 20-25 | 5-10 | 25-30 |
| 13 | 20-25 | 10-15 | 0-5 |
| 14 | 20-25 | 10-15 | 5-10 |
| 15 | 20-25 | 10-15 | 10-15 |
| 16 | 20-25 | 10-15 | 15-20 |
| 17 | 20-25 | 10-15 | 20-25 |
| 18 | 20-25 | 10-15 | 25-30 |
| 19 | 20-25 | 15-20 | 0-5 |
| 20 | 20-25 | 15-20 | 5-10 |
| 21 | 20-25 | 15-20 | 10-15 |
| 22 | 20-25 | 15-20 | 15-20 |
| 23 | 20-25 | 15-20 | 20-25 |
| 24 | 20-25 | 15-20 | 25-30 |
| 25 | 20-25 | 20-25 | 0-5 |
| 26 | 20-25 | 20-25 | 5-10 |
| 27 | 20-25 | 20-25 | 10-15 |
| 28 | 20-25 | 20-25 | 15-20 |
| 29 | 20-25 | 20-25 | 20-25 |
| 30 | 20-25 | 20-25 | 25-30 |
| 31 | 25-30 | 0-5 | 0-5 |
| 32 | 25-30 | 0-5 | 5-10 |
| 33 | 25-30 | 0-5 | 10-15 |
| 34 | 25-30 | 0-5 | 15-20 |
| 35 | 25-30 | 0-5 | 20-25 |
| 36 | 25-30 | 0-5 | 25-30 |
| 37 | 25-30 | 5-10 | 0-5 |
| 38 | 25-30 | 5-10 | 5-10 |
| 39 | 25-30 | 5-10 | 10-15 |
| 40 | 25-30 | 5-10 | 15-20 |
| 41 | 25-30 | 5-10 | 20-25 |
| 42 | 25-30 | 5-10 | 25-30 |
| 43 | 25-30 | 10-15 | 0-5 |
| 44 | 25-30 | 10-15 | 5-10 |
| 45 | 25-30 | 10-15 | 10-15 |
| 46 | 25-30 | 10-15 | 15-20 |
| 47 | 25-30 | 10-15 | 20-25 |
| 48 | 25-30 | 10-15 | 25-30 |
| 49 | 25-30 | 15-20 | 0-5 |
| 50 | 25-30 | 15-20 | 5-10 |
| 51 | 25-30 | 15-20 | 10-15 |
| 52 | 25-30 | 15-20 | 15-20 |
| 53 | 25-30 | 15-20 | 20-25 |
| 54 | 25-30 | 15-20 | 25-30 |
| 55 | 25-30 | 20-25 | 0-5 |
| 56 | 25-30 | 20-25 | 5-10 |
| 57 | 25-30 | 20-25 | 10-15 |
| 58 | 25-30 | 20-25 | 15-20 |
| 59 | 25-30 | 20-25 | 20-25 |
| 60 | 25-30 | 20-25 | 25-30 |
| 61 | 30-35 | 0-5 | 0-5 |
| 62 | 30-35 | 0-5 | 5-10 |
| 63 | 30-35 | 0-5 | 10-15 |
| 64 | 30-35 | 0-5 | 15-20 |
| 65 | 30-35 | 0-5 | 20-25 |
| 66 | 30-35 | 0-5 | 25-30 |
| 67 | 30-35 | 5-10 | 0-5 |
| 68 | 30-35 | 5-10 | 5-10 |
| 69 | 30-35 | 5-10 | 10-15 |
| 70 | 30-35 | 5-10 | 15-20 |
| 71 | 30-35 | 5-10 | 20-25 |
| 72 | 30-35 | 5-10 | 25-30 |
| 73 | 30-35 | 10-15 | 0-5 |
| 74 | 30-35 | 10-15 | 5-10 |
| 75 | 30-35 | 10-15 | 10-15 |
| 76 | 30-35 | 10-15 | 15-20 |
| 77 | 30-35 | 10-15 | 20-25 |
| 78 | 30-35 | 10-15 | 25-30 |
| 79 | 30-35 | 15-20 | 0-5 |
| 80 | 30-35 | 15-20 | 5-10 |
| 81 | 30-35 | 15-20 | 10-15 |
| 82 | 30-35 | 15-20 | 15-20 |
| 83 | 30-35 | 15-20 | 20-25 |
| 84 | 30-35 | 15-20 | 25-30 |
| 85 | 30-35 | 20-25 | 0-5 |
| 86 | 30-35 | 20-25 | 5-10 |
| 87 | 30-35 | 20-25 | 10-15 |
| 88 | 30-35 | 20-25 | 15-20 |
| 89 | 30-35 | 20-25 | 20-25 |
| 90 | 30-35 | 20-25 | 25-30 |
| 91 | 35-40 | 0-5 | 0-5 |
| 92 | 35-40 | 0-5 | 5-10 |
| 93 | 35-40 | 0-5 | 10-15 |
| 94 | 35-40 | 0-5 | 15-20 |
| 95 | 35-40 | 0-5 | 20-25 |
| 96 | 35-40 | 0-5 | 25-30 |
| 97 | 35-40 | 5-10 | 0-5 |
| 98 | 35-40 | 5-10 | 5-10 |
| 99 | 35-40 | 5-10 | 10-15 |
| 100 | 35-40 | 5-10 | 15-20 |
| 101 | 35-40 | 5-10 | 20-25 |
| 102 | 35-40 | 5-10 | 25-30 |
| 103 | 35-40 | 10-15 | 0-5 |
| 104 | 35-40 | 10-15 | 5-10 |
| 105 | 35-40 | 10-15 | 10-15 |
| 106 | 35-40 | 10-15 | 15-20 |
| 107 | 35-40 | 10-15 | 20-25 |
| 108 | 35-40 | 10-15 | 25-30 |
| 109 | 35-40 | 15-20 | 0-5 |
| 110 | 35-40 | 15-20 | 5-10 |
| 111 | 35-40 | 15-20 | 10-15 |
| 112 | 35-40 | 15-20 | 15-20 |
| 113 | 35-40 | 15-20 | 20-25 |
| 114 | 35-40 | 15-20 | 25-30 |
| 115 | 35-40 | 20-25 | 0-5 |
| 116 | 35-40 | 20-25 | 5-10 |
| 117 | 35-40 | 20-25 | 10-15 |
| 118 | 35-40 | 20-25 | 15-20 |
| 119 | 35-40 | 20-25 | 20-25 |
| 120 | 35-40 | 20-25 | 25-30 |
| 121 | 40-45 | 0-5 | 0-5 |
| 122 | 40-45 | 0-5 | 5-10 |
| 123 | 40-45 | 0-5 | 10-15 |
| 124 | 40-45 | 0-5 | 15-20 |
| 125 | 40-45 | 0-5 | 20-25 |
| 126 | 40-45 | 0-5 | 25-30 |
| 127 | 40-45 | 5-10 | 0-5 |
| 128 | 40-45 | 5-10 | 5-10 |
| 129 | 40-45 | 5-10 | 10-15 |
| 130 | 40-45 | 5-10 | 15-20 |
| 131 | 40-45 | 5-10 | 20-25 |
| 132 | 40-45 | 5-10 | 25-30 |
| 133 | 40-45 | 10-15 | 0-5 |
| 134 | 40-45 | 10-15 | 5-10 |
| 135 | 40-45 | 10-15 | 10-15 |
| 136 | 40-45 | 10-15 | 15-20 |
| 137 | 40-45 | 10-15 | 20-25 |
| 138 | 40-45 | 10-15 | 25-30 |
| 139 | 40-45 | 15-20 | 0-5 |
| 140 | 40-45 | 15-20 | 5-10 |
| 141 | 40-45 | 15-20 | 10-15 |
| 142 | 40-45 | 15-20 | 15-20 |
| 143 | 40-45 | 15-20 | 20-25 |
| 144 | 40-45 | 15-20 | 25-30 |
| 145 | 40-45 | 20-25 | 0-5 |
| 146 | 40-45 | 20-25 | 5-10 |
| 147 | 40-45 | 20-25 | 10-15 |
| 148 | 40-45 | 20-25 | 15-20 |
| 149 | 40-45 | 20-25 | 20-25 |
| 150 | 40-45 | 20-25 | 25-30 |
| 151 | >50 | 0-5 | 0-5 |
| 152 | >50 | 0-5 | 5-10 |
| 153 | >50 | 0-5 | 10-15 |
| 154 | >50 | 0-5 | 15-20 |
| 155 | >50 | 0-5 | 20-25 |
| 156 | >50 | 0-5 | 25-30 |
| 157 | >50 | 5-10 | 0-5 |
| 158 | >50 | 5-10 | 5-10 |
| 159 | >50 | 5-10 | 10-15 |
| 160 | >50 | 5-10 | 15-20 |
| 161 | >50 | 5-10 | 20-25 |
| 162 | >50 | 5-10 | 25-30 |
| 163 | >50 | 10-15 | 0-5 |
| 164 | >50 | 10-15 | 5-10 |
| 165 | >50 | 10-15 | 10-15 |
| 166 | >50 | 10-15 | 15-20 |
| 167 | >50 | 10-15 | 20-25 |
| 168 | >50 | 10-15 | 25-30 |
| 169 | >50 | 15-20 | 0-5 |
| 170 | >50 | 15-20 | 5-10 |
| 171 | >50 | 15-20 | 10-15 |
| 172 | >50 | 15-20 | 15-20 |
| 173 | >50 | 15-20 | 20-25 |
| 174 | >50 | 15-20 | 25-30 |
| 175 | >50 | 20-25 | 0-5 |
| 176 | >50 | 20-25 | 5-10 |
| 177 | >50 | 20-25 | 10-15 |
| 178 | >50 | 20-25 | 15-20 |
| 179 | >50 | 20-25 | 20-25 |
| 180 | >60 | 10-20 | 10-20 |
| 181 | >60 | 5-10 | 10-20 |
| 182 | >60 | 0-10 | 0-10 |
| 183 | >70 | 10-20 | 10-20 |
| 184 | >70 | 5-10 | 10-20 |
| 185 | >70 | 0-10 | 0-10 |
| 186 | >80 | 10-20 | 10-20 |
| 187 | >80 | 5-10 | 10-20 |
| 188 | >80 | 0-10 | 0-10 |
| 189 | >85 | 10-20 | 10-20 |
| 190 | >85 | 0-10 | 0-10 |
| 191 | >85 | 0-10 | 0-5 |
| 192 | >90 | 0-10 | 0-10 |

The yield of conversion for the one or more sugars to the one or more oligosaccharides in the methods described herein can be determined by any suitable method known in the art, including, for example, high performance liquid chromatography (HPLC). In some embodiments, the yield of conversion to one or more oligosaccharides to with DP>1 after combining the one or more sugars with the catalyst (e.g., at 2, 3, 4, 8, 12, 24, or 48 hours after combining the one or more sugars with the catalyst) is greater than about 50% (e.g., greater than about 55%, 60%, 65%, 70%, 75%, 80%, 85%, 90%, 95%, or 98%). In some embodiments, the yield of conversion to one or more oligosaccharides of >DP2 after combining the one or more sugars with the catalyst (e.g., at 2, 3, 4, 8, 12, 24, or 48 hours after combining the one or more sugars with the catalyst) is greater than 30% (e.g., greater than 35%, 40%, 45%, 50%, 55%. 60%, 65%, 70%, 75%, 80%, 85%, 90%, 95%, or 98%).

In some embodiments, the methods described herein to produce an oligosaccharide composition provide low levels of degradation products, resulting in relatively higher selectivity when compared to existing catalysts. The molar yield to sugar degradation products and selectivity may be determined by any suitable method known in the art, including, for example, HPLC. In some embodiments, the amount of sugar degradation products after combining the one or more sugars with the catalyst (e.g., at 2, 3, 4, 8, 12, 24, or 48 hours after combining the one or more sugars with the catalyst) is less than about 10% (e.g., less than about 9%, 8%, 7%, 6%, 5%, 4%, 3%, 2%, 1%, 0.75%, 0.5%, 0.25%, or 0.1%), such as less than about 10% of any one or combination of 1,6-anhydroglucose (levoglucosan), 5-hydroxymethylfurfural, 2-furaldehyde, acetic acid, formic acid, levulinic acid and/or humins. In some embodiments, the molar selectivity to oligosaccharide product after combining the one or more sugars with the catalyst (e.g., at 2, 3, 4, 8, 12, 24, or 48 hours after combining the one or more sugars with the catalyst) is greater than about 90% (e.g., greater than about 95%, 97%, 98%, 99%, 99.5%, or 99.9%).

In some variations, at least 10 dry wt % of the oligosaccharide composition produced according to the methods described herein has a degree of polymerization of at least 3. In some embodiments, at least 10 dry wt %, at least 20 dry wt %, at least 30 dry wt %, at least 40 dry wt %, at least 50 dry wt %, at least 60 dry wt %, at least 70 wt %, between 10 to 90 dry wt %, between 20 to 80 dry wt %, between 30 to 80 dry wt %, between 50 to 80 dry wt %, or between 70 to 80 dry wt % of the oligosaccharide composition has a degree of polymerization of at least 3.

In some variations, the oligosaccharide composition produced according to methods described herein has a DP3+ of at least 10% on a dry-weight basis. In certain variations, the oligosaccharide composition produced according to methods described herein has a DP3+ of at least 10% on a dry-weight basis, at least 20% on a dry-weight basis, at least 30% on a dry-weight basis, at least 40% on a dry-weight basis, at least 50% on a dry-weight basis, at least 60% on a dry-weight basis, at least 70% on a dry-weight basis, between 10 to 90% on a dry-weight basis, between 20 to 80% on a dry-weight basis, between 30 to 80% on a dry-weight basis, between 50 to 80% on a dry-weight basis, or between 70 to 80% on a dry-weight basis.

Glass Transition Temperature

In some variations, “glass transition” refers to the reversible transition of some compounds from a hard and relatively brittle state to a softer, flexible state. In some variations, “glass transition temperature” refers to the temperature determined by differential scanning calorimetry.

The glass transition temperature of a material can impart desirable characteristics to that material, and/or can impart desirable characteristics to a composition comprising that material. For example, varying the glass transition temperature of the oligosaccharide composition can affect its blendability in the animal feed composition. In some embodiments, the methods described herein are used to produce one or more oligosaccharides with a specific glass transition temperature, or within a glass transition temperature range. In some variations, the glass transition temperature of one or more oligosaccharides produced according to the methods described herein imparts desirable characteristics to the one or more oligosaccharides (e.g., texture, storage, or processing characteristics). In certain variations, the glass transition temperature of the one or more oligosaccharides imparts desirable characteristics to a composition including the one or more oligosaccharides (e.g., texture, storage, or processing characteristics).

For example, in some variations, therapeutic compositions that include the one or more oligosaccharides with a lower glass transition temperature have a softer texture than therapeutic compositions that include the one or more oligosaccharides with a higher glass transition temperature, or therapeutic compositions that do not include the one or more oligosaccharides. In other variations, therapeutic compositions including the one or more oligosaccharides with a higher glass transition temperature have reduced caking and can be dried at higher temperatures than therapeutic compositions including the one or more oligosaccharides with a lower glass transition temperature, or therapeutic compositions that do not include the one or more oligosaccharides.

In some embodiments, the glass transition temperature of the one or more oligosaccharides when prepared in a dry powder form with a moisture content below 6% is at least 0 degrees Celsius, at least 10 degrees Celsius, at least 20 degrees Celsius, at least 30 degrees Celsius, at least 40 degrees Celsius, at least 50 degrees Celsius, at least 60 degrees Celsius, at least 70 degrees Celsius, at least 80 degrees Celsius, at least 90 degrees Celsius, or at least 100 degrees Celsius. In certain embodiments, the glass transition temperature of the one or more oligosaccharides is between 40 degrees Celsius and 80 degrees Celsius.

Hygroscopicity

In some variations, “hygroscopicity” refers to the ability of a compound to attract and hold water molecules from the surrounding environment. The hygroscopicity of a material can impart desirable characteristics to that material, and/or can impart desirable characteristics to a composition comprising that material. In some embodiments, the methods described herein are used to produce one or more oligosaccharides with a specific hygroscopicity value or a range of hygroscopicity values. In some variations, the hygroscopicity of one or more oligosaccharides produced according to the methods described herein imparts desirable characteristics to the one or more oligosaccharides (e.g., texture, storage, or processing characteristics). In certain variations, the hygroscopicity of the one or more oligosaccharides imparts desirable characteristics to a composition including the one or more oligosaccharides (e.g., texture, storage, or processing characteristics).

For example, in some variations, therapeutic compositions that include the one or more oligosaccharides with a higher hygroscopicity have a softer texture than therapeutic compositions that include the one or more oligosaccharides with a lower hygroscopicity, or therapeutic compositions without the one or more oligosaccharides. In certain variations, the one or more oligosaccharides with a higher hygroscopicity are included in therapeutic compositions to reduce water activity, increase shelf life, produce a softer composition, produce a moister composition, and/or enhance the surface sheen of the composition.

In other variations, therapeutic compositions including the one or more oligosaccharides with a lower hygroscopicity have reduced caking and can be dried at a higher temperature than therapeutic compositions including the one or more oligosaccharides with a higher hygroscopicity, or therapeutic compositions without the one or more oligosaccharides. In certain variations, the one or more oligosaccharides with a lower hygroscopicity are included in therapeutic compositions to increase crispness, increase shelf life, reduce clumping, reduce caking, improve, and/or enhance the appearance of the composition.

The hygroscopicity of a composition, including the one or more oligosaccharides, can be determined by measuring the mass gain of the composition after equilibration in a fixed water activity atmosphere (e.g., a dessicator held at a fixed relative humidity).

In some embodiments, the hygroscopicity of the one or more oligosaccharides is at least 5% moisture content at a water activity of at least 0.6, at least 10% moisture content at a water activity of at least 0.6, at least 15% moisture content at a water activity of at least 0.6, at least 20% moisture content at a water activity of at least 0.6, or at least 30% moisture content at a water activity of at least 0.6. In certain embodiments, the hygroscopicity of the one or more oligosaccharides is between 5% moisture content and 15% moisture content at a water activity of at least 0.6.

In some embodiments, the mean degree of polymerization (DP), glass transition temperature (Tg), and hygroscopicity of the oligosaccharide composition produced by combining the one or more sugars with the catalyst (e.g., at 2, 3, 4, 8, 12, 24, or 48 hours after combining the one or more sugars with the catalyst) is any one of entries (1)-(180) of Table 1 B.

TABLE 1B

| | | Tg at <10 wt | Hygroscopicity |
|---|---|---|---|
| Number | Mean DP | % H2O (° C.) | (wt % H2O @ 0.6 Aw) |
| 1 | 5-10 | >50 | >5% |
| 2 | 5-10 | >50 | >5% |
| 3 | 5-10 | >50 | >5% |
| 4 | 5-10 | >50 | >5% |
| 5 | 5-10 | >50 | >5% |
| 6 | 5-10 | >50 | >10% |
| 7 | 5-10 | >50 | >10% |
| 8 | 5-10 | >50 | >10% |
| 9 | 5-10 | >50 | >10% |
| 10 | 5-10 | >50 | >10% |
| 11 | 5-10 | >50 | >15% |
| 12 | 5-10 | >50 | >15% |
| 13 | 5-10 | >50 | >15% |
| 14 | 5-10 | >50 | >15% |
| 15 | 5-10 | >50 | >15% |
| 16 | 5-10 | >50 | >5% |
| 17 | 5-10 | >50 | >5% |
| 18 | 5-10 | >50 | >5% |
| 19 | 5-10 | >50 | >5% |
| 20 | 5-10 | >50 | >5% |
| 21 | 5-10 | >50 | >10% |
| 22 | 5-10 | >50 | >10% |
| 23 | 5-10 | >50 | >10% |
| 24 | 5-10 | >50 | >10% |
| 25 | 5-10 | >50 | >10% |
| 26 | 5-10 | >50 | >15% |
| 27 | 5-10 | >50 | >15% |
| 28 | 5-10 | >50 | >15% |
| 29 | 5-10 | >50 | >15% |
| 30 | 5-10 | >50 | >15% |
| 31 | 5-10 | >75 | >5% |
| 32 | 5-10 | >75 | >5% |
| 33 | 5-10 | >75 | >5% |
| 34 | 5-10 | >75 | >5% |
| 35 | 5-10 | >75 | >5% |
| 36 | 5-10 | >75 | >10% |
| 37 | 5-10 | >75 | >10% |
| 38 | 5-10 | >75 | >10% |
| 39 | 5-10 | >75 | >10% |
| 40 | 5-10 | >75 | >10% |
| 41 | 5-10 | >75 | >15% |
| 42 | 5-10 | >75 | >15% |
| 43 | 5-10 | >75 | >15% |
| 44 | 5-10 | >75 | >15% |
| 45 | 5-10 | >75 | >15% |
| 46 | 5-10 | >75 | >5% |
| 47 | 5-10 | >75 | >5% |
| 48 | 5-10 | >75 | >5% |
| 49 | 5-10 | >75 | >5% |
| 50 | 5-10 | >75 | >5% |
| 51 | 5-10 | >75 | >10% |
| 52 | 5-10 | >75 | >10% |
| 53 | 5-10 | >75 | >10% |
| 54 | 5-10 | >75 | >10% |
| 55 | 5-10 | >75 | >10% |
| 56 | 5-10 | >75 | >15% |
| 57 | 5-10 | >75 | >15% |
| 58 | 5-10 | >75 | >15% |
| 59 | 5-10 | >75 | >15% |
| 60 | 5-10 | >75 | >15% |
| 61 | 5-10 | >100 | >5% |
| 62 | 5-10 | >100 | >5% |
| 63 | 5-10 | >100 | >5% |
| 64 | 5-10 | >100 | >5% |
| 65 | 5-10 | >100 | >5% |
| 66 | 5-10 | >100 | >10% |
| 67 | 5-10 | >100 | >10% |
| 68 | 5-10 | >100 | >10% |
| 69 | 5-10 | >100 | >10% |
| 70 | 5-10 | >100 | >10% |
| 71 | 5-10 | >100 | >15% |
| 72 | 5-10 | >100 | >15% |
| 73 | 5-10 | >100 | >15% |
| 74 | 5-10 | >100 | >15% |
| 75 | 5-10 | >100 | >15% |
| 76 | 5-10 | >100 | >5% |
| 77 | 5-10 | >100 | >5% |
| 78 | 5-10 | >100 | >5% |
| 79 | 5-10 | >100 | >5% |
| 80 | 5-10 | >100 | >5% |
| 81 | 5-10 | >100 | >10% |
| 82 | 5-10 | >100 | >10% |
| 83 | 5-10 | >100 | >10% |
| 84 | 5-10 | >100 | >10% |
| 85 | 5-10 | >100 | >10% |
| 86 | 5-10 | >100 | >15% |
| 87 | 5-10 | >100 | >15% |
| 88 | 5-10 | >100 | >15% |
| 89 | 5-10 | >100 | >15% |
| 90 | 5-10 | >100 | >15% |
| 91 | 10-15 | >50 | >5% |
| 92 | 10-15 | >50 | >5% |
| 93 | 10-15 | >50 | >5% |
| 94 | 10-15 | >50 | >5% |
| 95 | 10-15 | >50 | >5% |
| 96 | 10-15 | >50 | >10% |
| 97 | 10-15 | >50 | >10% |
| 98 | 10-15 | >50 | >10% |
| 99 | 10-15 | >50 | >10% |
| 100 | 10-15 | >50 | >10% |
| 101 | 10-15 | >50 | >15% |
| 102 | 10-15 | >50 | >15% |
| 103 | 10-15 | >50 | >15% |
| 104 | 10-15 | >50 | >15% |
| 105 | 10-15 | >50 | >15% |
| 106 | 10-15 | >50 | >5% |
| 107 | 10-15 | >50 | >5% |
| 108 | 10-15 | >50 | >5% |
| 109 | 10-15 | >50 | >5% |
| 110 | 10-15 | >50 | >5% |
| 111 | 10-15 | >50 | >10% |
| 112 | 10-15 | >50 | >10% |
| 113 | 10-15 | >50 | >10% |
| 114 | 10-15 | >50 | >10% |
| 115 | 10-15 | >50 | >10% |
| 116 | 10-15 | >50 | >15% |
| 117 | 10-15 | >50 | >15% |
| 118 | 10-15 | >50 | >15% |
| 119 | 10-15 | >50 | >15% |
| 120 | 10-15 | >50 | >15% |
| 121 | 10-15 | >75 | >5% |
| 122 | 10-15 | >75 | >5% |
| 123 | 10-15 | >75 | >5% |
| 124 | 10-15 | >75 | >5% |
| 125 | 10-15 | >75 | >5% |
| 126 | 10-15 | >75 | >10% |
| 127 | 10-15 | >75 | >10% |
| 128 | 10-15 | >75 | >10% |
| 129 | 10-15 | >75 | >10% |
| 130 | 10-15 | >75 | >10% |
| 131 | 10-15 | >75 | >15% |
| 132 | 10-15 | >75 | >15% |
| 133 | 10-15 | >75 | >15% |
| 134 | 10-15 | >75 | >15% |
| 135 | 10-15 | >75 | >15% |
| 136 | 10-15 | >75 | >5% |
| 137 | 10-15 | >75 | >5% |
| 138 | 10-15 | >75 | >5% |
| 139 | 10-15 | >75 | >5% |
| 140 | 10-15 | >75 | >5% |
| 141 | 10-15 | >75 | >10% |
| 142 | 10-15 | >75 | >10% |
| 143 | 10-15 | >75 | >10% |
| 144 | 10-15 | >75 | >10% |
| 145 | 10-15 | >75 | >10% |
| 146 | 10-15 | >75 | >15% |
| 147 | 10-15 | >75 | >15% |
| 148 | 10-15 | >75 | >15% |
| 149 | 10-15 | >75 | >15% |
| 150 | 10-15 | >75 | >15% |
| 151 | 10-15 | >100 | >5% |
| 152 | 10-15 | >100 | >5% |
| 153 | 10-15 | >100 | >5% |
| 154 | 10-15 | >100 | >5% |
| 155 | 10-15 | >100 | >5% |
| 156 | 10-15 | >100 | >10% |
| 157 | 10-15 | >100 | >10% |
| 158 | 10-15 | >100 | >10% |
| 159 | 10-15 | >100 | >10% |
| 160 | 10-15 | >100 | >10% |
| 161 | 10-15 | >100 | >15% |
| 162 | 10-15 | >100 | >15% |
| 163 | 10-15 | >100 | >15% |
| 164 | 10-15 | >100 | >15% |
| 165 | 10-15 | >100 | >15% |
| 166 | 10-15 | >100 | >5% |
| 167 | 10-15 | >100 | >5% |
| 168 | 10-15 | >100 | >5% |
| 169 | 10-15 | >100 | >5% |
| 170 | 10-15 | >100 | >5% |
| 171 | 10-15 | >100 | >10% |
| 172 | 10-15 | >100 | >10% |
| 173 | 10-15 | >100 | >10% |
| 174 | 10-15 | >100 | >10% |
| 175 | 10-15 | >100 | >10% |
| 176 | 10-15 | >100 | >15% |
| 177 | 10-15 | >100 | >15% |
| 178 | 10-15 | >100 | >15% |
| 179 | 10-15 | >100 | >15% |
| 180 | 10-15 | >100 | >15% |

Glycosidic Bond Type Distribution

In certain variations, the oligosaccharide composition produced according to the methods described herein has a distribution of glycosidic bond linkages. The distribution of glycosidic bond types may be determined by any suitable methods known in the art, including, for example, proton NMR or two dimensional J-resolved nuclear magnetic resonance spectroscopy (2D-JRES NMR). In some variations, the distribution of glycosidic bond types described herein is determined by 2D-JRES NMR.

As described above, the oligosaccharide composition may comprise hexose sugar monomers (such as glucose) or pentose sugar monomers (such as xylose), or combinations thereof. It should be understood by one of skill in the art that certain types of glycosidic linkages may not be applicable to oligosaccharides comprising pentose sugar monomers.

In some variations, the oligosaccharide composition has a bond distribution with:
(i) α-(1,2) glycosidic linkages;
(ii) α-(1,3) glycosidic linkages;
(iii) α-(1,4) glycosidic linkages;
(iv) α-(1,6) glycosidic linkages;
(v) β-(1,2) glycosidic linkages;
(vi) β-(1,3) glycosidic linkages;
(vii) β-(1,4) glycosidic linkages; or
(viii) β-(1,6) glycosidic linkages,

or any combination of (i) to (viii) above.

For example, in some variations, the oligosaccharide composition has a bond distribution with a combination of (ii) and (vi) glycosidic linkages. In other variations, the oligosaccharide composition has a bond distribution with a combination of (i), (viii), and (iv) glycosidic linkages. In another variation, the oligosaccharide composition has a bond distribution with a combination of (i), (ii), (v), (vi), (vii), and (viii) glycosidic linkages.

In certain variations, the oligosaccharide composition has a bond distribution with any combination of (i), (ii), (iii), (v), (vi), and (vii) glycosidic linkages, and comprises oligosaccharides with pentose sugar monomers. In other variations, the oligosaccharide composition has a bond distribution with any combination of (i), (ii), (iii), (iv), (v), (vi), (vii) and (viii) glycosidic linkages, and comprises oligosaccharides with hexose sugar monomers. In still other variations, the oligosaccharide composition has a bond distribution with any combination of (i), (ii), (iii), (iv), (v), (vi), (vii) and (viii) glycosidic linkages, and comprises oligosaccharides with hexose sugar monomers, and oligosaccharides with pentose sugar monomers. In still other variations, the oligosaccharide composition has a bond distribution with any combination of (i), (ii), (iii), (iv), (v), (vi), (vii) and (viii) glycosidic linkages, and comprises oligosaccharides with hexose sugar monomers and pentose sugar monomers. In yet another variation, the oligosaccharide composition has a bond distribution with any combination of (i), (ii), (iii), (iv), (v), (vi), (vii) and (viii) glycosidic linkages, and comprises oligosaccharides with hexose sugar monomers, oligosaccharides with pentose sugar monomers, and oligosaccharides with hexose and pentose sugar monomers.

In some variations, the oligosaccharide composition has a glycosidic bond type distribution of less than 20 mol % α-(1,2) glycosidic linkages, less than 10 mol % α-(1,2) glycosidic linkages, less than 5 mol % α-(1,2) glycosidic linkages, between 0 to 25 mol % α-(1,2) glycosidic linkages, between 1 to 25 mol % α-(1,2) glycosidic linkages, between 0 to 20 mol % α-(1,2) glycosidic linkages, between 1 to 15 mol % α-(1,2) glycosidic linkages, between 0 to 10 mol % α-(1,2) glycosidic linkages, or between 1 to 10 mol % α-(1,2) glycosidic linkages.

In some variations, the oligosaccharide composition has a glycosidic bond type distribution of less than 50 mol % β-(1,2) glycosidic linkages, less than 40 mol % β-(1,2) glycosidic linkages, less than 35 mol % β-(1,2) glycosidic linkages, less than 30 mol % β-(1,2) glycosidic linkages, less than 25 mol % β-(1,2) glycosidic linkages, less than 10 mol % β-(1,2) glycosidic linkages, at least 1 mol % β-(1,2) glycosidic linkages, at least 5 mol % β-(1,2) glycosidic linkages, at least 10 mol % β-(1,2) glycosidic linkages, at least 15 mol % β-(1,2) glycosidic linkages, at least 20 mol % β-(1,2) glycosidic linkages, between 0 to 30 mol % β-(1,2) glycosidic linkages, between 1 to 30 mol % β-(1,2) glycosidic linkages, between 0 to 25 mol % β-(1,2) glycosidic linkages, between 1 to 25 mol % β-(1,2) glycosidic linkages, between 10 to 30 mol % β-(1,2) glycosidic linkages, between 15 to 25 mol % β-(1,2) glycosidic linkages, between 0 to 10 mol % β-(1,2) glycosidic linkages, between 1 to 10 mol % β-(1,2) glycosidic linkages, between 10 to 50 mol % β-(1,2) glycosidic linkages, between 10 to 40 mol % β-(1,2) glycosidic linkages, between 20 to 35 mol % β-(1,2) glycosidic linkages, between 20 to 35 mol % β-(1,2) glycosidic linkages, between 20 to 50 mol % β-(1,2) glycosidic linkages, between 30 to 40 mol % β-(1,2) glycosidic linkages, between 10 to 30 mol % β-(1,2) glycosidic linkages, or between 10 to 20 mol % β-(1,2) glycosidic linkages.

In some variations, the oligosaccharide composition has a glycosidic bond type distribution of less than 40 mol % α-(1,3) glycosidic linkages, less than 30 mol % α-(1,3) glycosidic linkages, less than 25 mol % α-(1,3) glycosidic linkages, less than 20 mol % α-(1,3) glycosidic linkages, less than 15 mol % α-(1,3) glycosidic linkages, at least 1 mol % α-(1,3) glycosidic linkages, at least 5 mol % α-(1,3) glycosidic linkages, at least 10 mol % α-(1,3) glycosidic linkages, at least 15 mol % α-(1,3) glycosidic linkages, at least 20 mol % α-(1,3) glycosidic linkages, at least 25 mol % α-(1,3) glycosidic linkages, between 0 to 30 mol % α-(1,3) glycosidic linkages, between 1 to 30 mol % α-(1,3) glycosidic linkages, between 5 to 30 mol % α-(1,3) glycosidic linkages, between 10 to 25 mol % α-(1,3) glycosidic linkages, between 1 to 20 mol % α-(1,3) glycosidic linkages, or between 5 to 15 mol % α-(1,3) glycosidic linkages.

In some variations, the oligosaccharide composition has a glycosidic bond type distribution of less than 25 mol % β-(1,3) glycosidic linkages, less than 20 mol % β-(1,3) glycosidic linkages, less than 15 mol % β-(1,3) glycosidic linkages, less than 10 mol % β-(1,3) glycosidic linkages, at least 1 mol % β-(1,3) glycosidic linkages, at least 2 mol % β-(1,3) glycosidic linkages, at least 5 mol % β-(1,3) glycosidic linkages, at least 10 mol % β-(1,3) glycosidic linkages, at least 15 mol % β-(1,3) glycosidic linkages, between 1 to 20 mol % β-(1,3) glycosidic linkages, between 5 to 15 mol % β-(1,3) glycosidic linkages, between 1 to 15 mol % β-(1,3) glycosidic linkages, or between 2 to 10 mol % β-(1,3) glycosidic linkages.

In some variations, the oligosaccharide composition has a glycosidic bond type distribution of less than 20 mol % α-(1,4) glycosidic linkages, less than 15 mol % α-(1,4) glycosidic linkages, less than 10 mol % α-(1,4) glycosidic linkages, less than 9 mol % α-(1,4) glycosidic linkages, between 1 to 20 mol % α-(1,4) glycosidic linkages, between 1 to 15 mol % α-(1,4) glycosidic linkages, between 2 to 15 mol % α-(1,4) glycosidic linkages, between 5 to 15 mol % α-(1,4) glycosidic linkages, between 1 to 15 mol % α-(1,4) glycosidic linkages, or between 1 to 10 mol % α-(1,4) glycosidic linkages.

In some variations, the oligosaccharide composition has a glycosidic bond type distribution of less than 55 mol % β-(1,4) glycosidic linkages, less than 50 mol % β-(1,4) glycosidic linkages, less than 45 mol % β-(1,4) glycosidic linkages, less than 40 mol % β-(1,4) glycosidic linkages, less than 35 mol % β-(1,4) glycosidic linkages, less than 25 mol % β-(1,4) glycosidic linkages, less than 15 mol % β-(1,4) glycosidic linkages, less than 10 mol % β-(1,4) glycosidic linkages, at least 1 mol % β-(1,4) glycosidic linkages, at least 5 mol % β-(1,4) glycosidic linkages, at least 10 mol % β-(1,4) glycosidic linkages, at least 20 mol % β-(1,4) glycosidic linkages, at least 30 mol % β-(1,4) glycosidic linkages, between 0 to 55 mol % β-(1,4) glycosidic linkages, between 5 to 55 mol % β-(1,4) glycosidic linkages, between 10 to 50 mol % β-(1,4) glycosidic linkages, between 0 to 40 mol % β-(1,4) glycosidic linkages, between 1 to 40 mol % β-(1,4) glycosidic linkages, between 0 to 35 mol % β-(1,4) glycosidic linkages, between 1 to 35 mol % β-(1,4) glycosidic linkages, between 1 to 30 mol % β-(1,4) glycosidic linkages, between 5 to 25 mol % β-(1,4) glycosidic linkages, between 10 to 25 mol % β-(1,4) glycosidic linkages, between 15 to 25 mol % β-(1,4) glycosidic linkages, between 0 to 15 mol % β-(1,4) glycosidic linkages, between 1 to 15 mol % β-(1,4) glycosidic linkages, between 0 to 10 mol % β-(1,4) glycosidic linkages, or between 1 to 10 mol % β-(1,4) glycosidic linkages.

In some variations, the oligosaccharide composition has a glycosidic bond type distribution of less than 30 mol % α-(1,6) glycosidic linkages, less than 25 mol % α-(1,6) glycosidic linkages, less than 20 mol % α-(1,6) glycosidic linkages, less than 19 mol % α-(1,6) glycosidic linkages, less than 15 mol % α-(1,6) glycosidic linkages, less than 10 mol % α-(1,6) glycosidic linkages, between 0 to 30 mol % α-(1,6) glycosidic linkages, between 1 to 30 mol % α-(1,6) glycosidic linkages, between 5 to 25 mol % α-(1,6) glycosidic linkages, between 0 to 25 mol % α-(1,6) glycosidic linkages, between 1 to 25 mol % α-(1,6) glycosidic linkages, between 0 to 20 mol % α-(1,6) glycosidic linkages, between 0 to 15 mol % α-(1,6) glycosidic linkages, between 1 to 15 mol % α-(1,6) glycosidic linkages, between 0 to 10 mol % α-(1,6) glycosidic linkages, or between 1 to 10 mol % α-(1,6) glycosidic linkages. In some embodiments, the oligosaccharide composition comprises oligosaccharides with hexose sugar monomers.

In some variations, the oligosaccharide composition has a glycosidic bond type distribution of less than 55 mol % β-(1,6) glycosidic linkages, less than 50 mol % β-(1,6) glycosidic linkages, less than 35 mol % β-(1,6) glycosidic linkages, less than 30 mol % β-(1,6) glycosidic linkages, at least 1 mol % β-(1,6) glycosidic linkages, at least 5 mol % β-(1,6) glycosidic linkages, at least 10 mol % β-(1,6) glycosidic linkages, at least 15 mol % β-(1,6) glycosidic linkages, at least 20 mol % β-(1,6) glycosidic linkages, at least 25 mol % β-(1,6) glycosidic linkages, at least 20 mol % β-(1,6) glycosidic linkages, at least 25 mol % β-(1,6) glycosidic linkages, at least 30 mol % β-(1,6) glycosidic linkages, between 10 to 55 mol % β-(1,6) glycosidic linkages, between 5 to 55 mol % β-(1,6) glycosidic linkages, between 15 to 55 mol % β-(1,6) glycosidic linkages, between 20 to 55 mol % β-(1,6) glycosidic linkages, between 20 to 50 mol % β-(1,6) glycosidic linkages, between 25 to 55 mol % β-(1,6) glycosidic linkages, between 25 to 50 mol % β-(1,6) glycosidic linkages, between 5 to 40 mol % β-(1,6) glycosidic linkages, between 5 to 30 mol % β-(1,6) glycosidic linkages, between 10 to 35 mol % β-(1,6) glycosidic linkages, between 5 to 20 mol % β-(1,6) glycosidic linkages, between 5 to 15 mol % β-(1,6) glycosidic linkages, between 8 to 15 mol % β-(1,6) glycosidic linkages, or between 15 to 30 mol % β-(1,6) glycosidic linkages. In some embodiments, the oligosaccharide composition comprises oligosaccharides with hexose sugar monomers.

In some variations, the oligosaccharide composition has a glycosidic bond type distribution of at least 1 mol % α-(1,3) glycosidic linkages. In some variations, the oligosaccharide composition has a glycosidic bond type distribution of at least 10 mol % α-(1,3) glycosidic linkages.

In some variations, the oligosaccharide composition has a glycosidic bond type distribution of at least 1 mol % β-(1,3) glycosidic linkages. In some variations, the oligosaccharide composition has a glycosidic bond type distribution of at least 10 mol % β-(1,3) glycosidic linkages.

In some variations, the oligosaccharide composition has a glycosidic bond type distribution of at least 15 mol % β-(1,6) glycosidic linkages. In some variations, the oligosaccharide composition has a glycosidic bond type distribution of at least 10 mol % β-(1,6) glycosidic linkages.

In some variations, the oligosaccharide composition has a glycosidic bond type distribution of at least 15 mol % β-(1,2) glycosidic linkages. In some variations, the oligosaccharide composition has a glycosidic bond type distribution of at least 10 mol % β-(1,2) glycosidic linkages.

It should be understood that the glycosidic linkage distributions described herein for the various types of linkages (e.g., α-(1,2), α-(1,3), α-(1,4), α-(1,6), β-(1,2), β-(1,3), β-(1,4), or β-(1,6) glycosidic linkages) may be combined as if each and every combination were individually listed, as applicable.

In some variations, the distribution of glycosidic bond types described above for any of the oligosaccharide compositions herein is determined by two dimensional J-resolved nuclear magnetic resonance (2D-JRES NMR) spectroscopy.

In certain variations, the oligosaccharide composition comprises only hexose sugar monomers, and has any glycosidic bond type distribution as described herein. In some variations, the oligosaccharide composition comprises only pentose sugar monomers, and has any glycosidic bond type distribution as described herein, as applicable. In yet other variations, the oligosaccharide composition comprises both pentose and hexose sugar monomers, and has any glycosidic bond type distribution as described herein, as applicable.

It should be further understood that variations for the type of oligosaccharides present in the composition, as well as the degree of polymerization, glass transition temperature, and hygroscopicity of the oligosaccharide composition, may be combined as if each and every combination were listed separately. For example, in some variations, the oligosaccharide composition is made up of a plurality of oligosaccharides, wherein the composition has a glycosidic bond distribution of:

at least 1 mol % α-(1,3) glycosidic linkages;

at least 1 mol % β-(1,3) glycosidic linkages;

at least 15 mol % β-(1,6) glycosidic linkages;

less than 20 mol % α-(1,4) glycosidic linkages; and

less than 30 mol % α-(1,6) glycosidic linkages, and

wherein at least 10 dry wt % of the oligosaccharide composition has a degree of polymerization of at least 3. In some variations, at least 50 dry wt %, or between 65 to 80 dry wt % of the oligosaccharide composition has a degree of polymerization of at least 3.

For example, in some variations, the oligosaccharide composition has a glycosidic bond type distribution of less than 20 mol % α-(1,4) glycosidic linkages, and less than 30 mol % α-(1,6) glycosidic linkages. In some variations, at least 10 dry wt % of the oligosaccharide composition has a degree of polymerization of at least 3. In some variations, at least 50 dry wt %, or between 65 to 80 dry wt % of the oligosaccharide composition has a degree of polymerization of at least 3.

In another variation, the oligosaccharide composition comprises a glycosidic bond type distribution of between 0 to 15 mol % α-(1,2) glycosidic linkages; between 0 to 30 mol % β-(1,2) glycosidic linkages; between 1 to 30 mol % α-(1,3) glycosidic linkages; between 1 to 20 mol % β-(1,3) glycosidic linkages; between 0 to 55 mol % β-(1,4) glycosidic linkages; and between 15 to 55 mol % β-(1,6) glycosidic linkages. In some variations, at least 10 dry wt % of the oligosaccharide composition has a degree of polymerization of at least 3. In some variations, at least 50 dry wt %, or between 65 to 80 dry wt % of the oligosaccharide composition has a degree of polymerization of at least 3.

In yet another variation, the oligosaccharide composition has a glycosidic bond type distribution of between 0 to 15 mol % α-(1,2) glycosidic linkages; between 10 to 30 mol % β-(1,2) glycosidic linkages; between 5 to 30 mol % α-(1,3) glycosidic linkages; between 1 to 20 mol % β-(1,3) glycosidic linkages; between 0 to 15 mol % β-(1,4) glycosidic linkages; between 20 to 55 mol % β-(1,6) glycosidic linkages; less than 20 mol % α-(1,4) glycosidic linkages; and less than 15 mol % α-(1,6) glycosidic linkages. In some variations, at least 10 dry wt % of the oligosaccharide composition has a degree of polymerization of at least 3. In some variations, at least 50 dry wt %, or between 65 to 80 dry wt % of the oligosaccharide composition has a degree of polymerization of at least 3.

In still other variations, the oligosaccharide composition has a glycosidic bond type distribution of between 0 to 10 mol % α-(1,2) glycosidic linkages, between 15 to 25 mol % β-(1,2) glycosidic linkages, between 10 to 25 mol % α-(1,3) glycosidic linkages, between 5 to 15 mol % β-(1,3) glycosidic linkages, between 5 to 15 mol % α-(1,4) glycosidic linkages, between 0 to 10 mol % β-(1,4) glycosidic linkages, between 0 to 10 mol % α-(1,6) glycosidic linkages, and between 25 to 50 mol % β-(1,6) glycosidic linkages. In some variations, at least 10 dry wt % of the oligosaccharide composition has a degree of polymerization of at least 3. In some variations, at least 50 dry wt %, or between 65 to 80 dry wt % of the oligosaccharide composition has a degree of polymerization of at least 3.

In certain variations, the oligosaccharide composition has a glycosidic bond type distribution of between 0 to 15 mol % α-(1,2) glycosidic linkages; between 0 to 15 mol % β-(1,2) glycosidic linkages; between 1 to 20 mol % α-(1,3) glycosidic linkages; between 1 to 15 mol % β-(1,3) glycosidic linkages; between 5 to 55 mol % β-(1,4) glycosidic linkages; between 15 to 55 mol % β-(1,6) glycosidic linkages; less than 20 mol % α-(1,4) glycosidic linkages; and less than 30 mol % α-(1,6) glycosidic linkages. In some variations, at least 10 dry wt % of the oligosaccharide composition has a degree of polymerization of at least 3. In some variations, at least 50 dry wt %, or between 65 to 80 dry wt % of the oligosaccharide composition has a degree of polymerization of at least 3.

In yet other variations, the oligosaccharide composition has a glycosidic bond type distribution of between 0 to 10 mol % α-(1,2) glycosidic linkages, between 0 to 10 mol % β-(1,2) glycosidic linkages, between 5 to 15 mol % α-(1,3) glycosidic linkages, between 2 to 10 mol % β-(1,3) glycosidic linkages, between 2 to 15 mol % α-(1,4) glycosidic linkages, between 10 to 50 mol % β-(1,4) glycosidic linkages, between 5 to 25 mol % α-(1,6) glycosidic linkages, and between 20 to 50 mol % β-(1,6) glycosidic linkages. In some variations, at least 10 dry wt % of the oligosaccharide composition has a degree of polymerization of at least 3. In some variations, at least 50 dry wt %, or between 65 to 80 dry wt % of the oligosaccharide composition has a degree of polymerization of at least 3.

In other variations, the oligosaccharide composition has a glycosidic bond type distribution of between 0 to 15 mol % α-(1,2) glycosidic linkages, between 0 to 30 mol % β-(1,2) glycosidic linkages, between 5 to 30 mol % α-(1,3) glycosidic linkages, between 1 to 20 mol % β-(1,3) glycosidic linkages, between 1 to 20 mol % α-(1,4) glycosidic linkages, between 0 to 40 mol % β-(1,4) glycosidic linkages, between 0 to 25 mol % α-(1,6) glycosidic linkages, and between 10 to 35 mol % β-(1,6) glycosidic linkages. In some variations, at least 10 dry wt % of the oligosaccharide composition has a degree of polymerization of at least 3. In some variations, at least 50 dry wt %, or between 65 to 80 dry wt % of the oligosaccharide composition has a degree of polymerization of at least 3.

In still other variations, the oligosaccharide composition has a glycosidic bond type distribution of between 0 to 10 mol % α-(1,2) glycosidic linkages, between 0 to 25 mol % β-(1,2) glycosidic linkages, between 10 to 25 mol % α-(1,3) glycosidic linkages, between 5 to 15 mol % β-(1,3) glycosidic linkages, between 5 to 15 mol % α-(1,4) glycosidic linkages, between 0 to 35 mol % β-(1,4) glycosidic linkages, between 0 to 20 mol % α-(1,6) glycosidic linkages, and between 15 to 30 mol % β-(1,6) glycosidic linkages. In some variations, at least 10 dry wt % of the oligosaccharide composition has a degree of polymerization of at least 3. In some variations, at least 50 dry wt %, or between 65 to 80 dry wt % of the oligosaccharide composition has a degree of polymerization of at least 3.

In still other variations, the oligosaccharide composition has a glycosidic bond type distribution of at least 1 mol % α-(1,3) glycosidic linkages, and at least 1 mol % β-(1,3) glycosidic linkages, wherein at least 10 dry wt % of the oligosaccharide composition has a degree of polymerization of at least 3. In some variations, the oligosaccharide composition further has a glycosidic bond type distribution of at least 15 mol % β-(1,6) glycosidic linkages. In yet other variations, at least 50 dry wt %, or between 65 to 80 dry wt % of the oligosaccharide composition has a degree of polymerization of at least 3.

In some variations, the oligosaccharide composition has a glycosidic bond type distribution of at least 10 mol % α-(1,3) glycosidic linkages; and at least 10 mol % β-(1,3) glycosidic linkages. In some variations, the oligosaccharide composition has a glycosidic bond type distribution of less than 9 mol % α-(1,4) glycosidic linkages; and less than 19 mol % α-(1,6) glycosidic linkages. In some variations, the oligosaccharide composition further has a glycosidic bond type distribution of at least 15 mol % β-(1,2) glycosidic linkages.

In other variations, the oligosaccharide composition has a glycosidic bond type distribution of less than 9 mol % α-(1,4) glycosidic linkages, and less than 19 mol % α-(1,6) glycosidic linkages.

In still other variations, the oligosaccharide composition has a glycosidic bond type distribution of between 0 to 20 mol % α-(1,2) glycosidic linkages; between 10 to 45 mol % β-(1,2) glycosidic linkages; between 1 to 30 mol % α-(1,3) glycosidic linkages; between 1 to 20 mol % β-(1,3) glycosidic linkages; between 0 to 55 mol % β-(1,4) glycosidic linkages; and between 10 to 55 mol % β-(1,6) glycosidic linkages.

In some variations, the oligosaccharide composition has a glycosidic bond type distribution of between 10 to 20 mol % α-(1,2) glycosidic linkages, between 23 to 31 mol % β-(1,2) glycosidic linkages, between 7 to 9 mol % α-(1,3) glycosidic linkages, between 4 to 6 mol % β-(1,3) glycosidic linkages, between 0 to 2 mol % α-(1,4) glycosidic linkages, between 18 to 22 mol % β-(1,4) glycosidic linkages, between 9 to 13 mol % α-(1,6) glycosidic linkages, and between 14 to 16 mol % β-(1,6) glycosidic linkages

In yet other variations, the oligosaccharide composition has a glycosidic bond type distribution of between 10 to 12 mol % α-(1,2) glycosidic linkages, between 31 to 39 mol % β-(1,2) glycosidic linkages, between 5 to 7 mol % α-(1,3) glycosidic linkages, between 2 to 4 mol % β-(1,3) glycosidic linkages, between 0 to 2 mol % α-(1,4) glycosidic linkages, between 19 to 23 mol % β-(1,4) glycosidic linkages, between 13 to 17 mol % α-(1,6) glycosidic linkages, and between 7 to 9 mol % β-(1,6) glycosidic linkages.

In some embodiments, which may be combined with any of the foregoing embodiments, at least 10 dry wt % of the oligosaccharide composition has a degree of polymerization of at least 3. In some variations, at least 50 dry wt %, or between 65 to 80 dry wt % of the oligosaccharide composition has a degree of polymerization of at least 3.

Delivery Vehicle

In some variations, the delivery vehicle degrades in the presence of certain enzymes present in specific regions of the gastrointestinal tract; or enables prolonged retention at specific regions of the gastrointestinal tract; or adheres to the mucosal surfaces of specific regions of the gastrointestinal tract; or increases in size in specific regions of the gastrointestinal tract to slow its passage through such regions; or floats or sinks in gastric fluids to alter the rate at which the composition is emptied from the stomach; or responds to certain conditions (e.g., pH conditions, pressure conditions) in the gastrointestinal tract; or any combinations of the foregoing. In one variation, the delivery vehicle is pH-sensitive, and is stable in the acidic pH of the stomach but dissolves in the neutral/alkaline conditions further along the gastrointestinal tract.

In some variations, the compositions described herein are formulated to deliver the carbohydrates and sugars to specific regions of the gastrointestinal tract in the animals and/or modulate at least a portion of the gut microbiome in the animals to improve animal health. Thus, in some aspects, provided is a therapeutic composition comprising:

(a) at least one carbohydrate, or at least one deoxy sugar, or at least one amino sugar, or at least one sugar alcohol, or at least one sugar acid, or at least one phosphate sugar, or at least one sulfate sugar, or a compound comprising 2 to 5 units, wherein each unit is independently a carbohydrate unit, a deoxy sugar unit, an amino sugar unit, a sugar alcohol unit, a sugar acid unit, a phosphate sugar unit, or a sulfate sugar unit, or any combinations of the foregoing; and

(b) a delivery vehicle.

In certain variations, the compositions are formulated to deliver the carbohydrates and sugars to specific regions of the gastrointestinal tract in the animals where digestibility of the carbohydrates and sugars are maximized. For example, such specific regions of the gastrointestinal tract in animals include the ileum and/or cecum.

In some variations, the delivery vehicle degrades in the presence of certain enzymes present in specific regions of the gastrointestinal tract; or enables prolonged retention at specific regions of the gastrointestinal tract; or adheres to the mucosal surfaces of specific regions of the gastrointestinal tract; or increases in size in specific regions of the gastrointestinal tract to slow its passage through such regions; or floats or sinks in gastric fluids to alter the rate at which the composition is emptied from the stomach; or responds to certain conditions (e.g., pH conditions, pressure conditions) in the gastrointestinal tract; or any combinations of the foregoing. In one variation, the delivery vehicle is pH-sensitive, and is stable in the acidic pH of the stomach but dissolves in the neutral/alkaline conditions further along the gastrointestinal tract.

In other variation, the delivery vehicle is an enzyme-responsive polymer, such as a trypsin-responsive polymer or a pepsin-responsive polymer that degrades in the presence of trypsin or pepsin, respectively, present in certain regions of the gastrointestinal tract in animals. In another variation, the delivery vehicle is a polyacrylic acid (including, for example, a cross-linked polyacrylic acid), a polycarbophil, a polyolefin, a polyamide, a polyurethane, carboxymethyl cellulose, hydroxypropyl cellulose, alginate, a carrageenan, a supramolecular polymer gel, a collagen sponge, a hydrogel (e.g.

hydroxyl propyl methyl cellulose (HPMC), poly methyl methacrylate or polyvinyl acetate), a superporous hydrogel composite, a hydrocolloid (e.g., hydroxypropyl methylcellulose), glycerol monooleate, chitosan, pectin, guar gum, inulin, cyclodextrin, dextran, amylase, chondrotin sulphate, or locust bean gum, or any combinations thereof.

The delivery vehicle may be in various forms. In some variations, the delivery vehicle may be one or more coatings for the carbohydrates and sugars. In other variations, the delivery is in the form of a matrix in which the carbohydrates and sugars are dispersed; or a pill, which may include a tablet, a capsule, a microneedle pill, that incorporates the carbohydrates and sugars. The release profile of the composition may be adjusted by varying the thickness, size or density of the vehicle.

Pharmaceutical Acceptable Vehicles

In some embodiments, the therapeutic compositions described herein comprise any of the oligosaccharide compositions described herein and at least one pharmaceutically acceptable vehicle. Pharmaceutically acceptable vehicles may include pharmaceutically acceptable carriers, adjuvants and/or excipients, and other ingredients can be deemed pharmaceutically acceptable insofar as they are compatible with other ingredients of the formulation and not deleterious to the recipient thereof.

As used herein, by “pharmaceutically acceptable” refers to a material that is not biologically or otherwise undesirable, e.g., the material may be incorporated into a pharmaceutical composition administered to an animal without causing any significant undesirable biological effects or interacting in a deleterious manner with any of the other components of the composition in which it is contained.

In certain variations, the compositions further comprise suitable pharmaceutically acceptable vehicles, which may include, for example, one or more fillers, excipients, binders, diluents, lubricants, disintegrants, glidants, stabilizers, surfactants, foaming agents, permeation enhancers, solubilizers, colorants, flavorants, or adjuvants, or any combinations thereof.

Examples of diluents may include cellulose, microcrystalline cellulose, dry starch, hydrolyzed starches, talc, sodium chloride, silicon dioxide, titanium oxide, dicalcium phosphate dihydrate, calcium sulfate, calcium carbonate, alumina, kaolin, ground corn meal, ground wheat meal, corn flour, wheat flow, ground rice hulls, diatomaceous earth, bentonite, kaolinite, vermiculum. Examples of binders may include starch (e.g., corn starch and pregelatinized starch), gelatin, cellulose, polyethylene glycol, wax, natural and synthetic gum (e.g., acacia, tragacanth), sodium alginate, and synthetic polymers (e.g., polymethacrylates and polyvinylpyrrolidone). Examples of lubricants may include magnesium stearate, calcium stearate, stearic acid, glyceryl behenate, and polyethylene glycol. Examples of disintegrants may include starches, alginic acid, crosslinked polymers (e.g., crosslinked polyvinylpyrrolidone, croscarmellose sodium), potassium or sodium starch glycolate, clays, celluloses, and gums. Examples of glidants may include silicon dioxide and talc. Examples of foaming agents may include sodium hydrogencarbonate, sodium carbonate, and calcium carbonate.

In certain variations, the therapeutic compositions described herein may further comprise acacia, alginate, alginic acid, aluminum acetate, benzyl alcohol, butyl paraben, butylated hydroxy toluene, calcium carbonate, calcium disodium EDTA, calcium hydrogen phosphate dihydrate, dibasic calcium phosphate, tribasic calcium phosphate, calcium stearate, candelilla wax, carboxymethylcellulose calcium, carnuba wax, castor oil hydrogenated, cellulose, cetylpyridine chloride, citric acid, colloidal silicone dioxide, copolyvidone, corn starch, croscarmellose sodium, crospovidone, cysteine HCl, dimethicone, disodium hydrogen phosphate, erythrosine sodium, ethyl cellulose, gelatin, glycerin, glyceryl behenate, glyceryl monooleate, glyceryl monostearate, glycine, HPMC pthalate, hydroxy propyl cellulose, hydroxyl propyl methyl cellulose, hypromellose, iron oxide red or ferric oxide, iron oxide yellow, iron oxide or ferric oxide, magnesium carbonate, magnesium oxide, magnesium stearate, methionine, methacrylic acid copolymer, methyl cellulose, methyl paraben, microcrystalline cellulose, silicified microcrystalline cellulose, mineral oil, polyethylene glycol (PEG), phosphoric acid, plain calcium phosphate, anhydrous calcium phosphate, poloxamer 407, poloxamer 188, plain poloxamer, polyethylene oxide, polyoxyl 40 stearate, polysorbate 80, potassium bicarbonate, potassium sorbate, potato starch, povidone, polyvinypyrrolidone (PVP), propylene glycol, propylene paraben, propyl paraben, retinyl palmitate, saccharin sodium, selenium, silica, silica gel, fumed silica, silicon dioxide, sodium alginate, sodium benzoate, sodium carbonate, sodium carboxymethyl cellulose, sodium chloride, sodium citrate dihydrate, sodium croscarmellose, sodium lauryl sulfate, sodium metabisulfite, sodium propionate, sodium starch, sodium starch glycolate, sodium stearyl fumarate, starch, pregelatinized starch, stearic acid, succinic acid, talc, titanium dioxide, triacetin, triethyl citrate, or vegetable stearin, or a combination thereof.

It should be understood that any variations of the oligosaccharide compositions described herein may be combined with any of the organic acids, the aromatic compounds, the probiotics, other therapeutic agents, and the pharmaceutical acceptable vehicles, as if each and every combination was individually listed. For example, in certain embodiments, the composition comprises a gluco-oligosaccharide, and a short-chain fatty acid. Such composition may be formulated as a tablet with one or more polymeric coatings to respond to certain pH conditions of the gastrointestinal tract in an animal. In another example, the composition comprises an oligosaccharide composition having a glycosidic bond type distribution of less than 55 mol % β-(1,6) glycosidic linkages, propionic acid, and resorcinol. Such composition may be formulated with a polymer that adheres to the mucosal surfaces of specific regions of the gastrointestinal tract in an animal.

Organic Acids

In some embodiments, the therapeutic compositions described herein may further comprise at least one organic acid. The organic acid may include, for example, acetic acid, propionic acid, butryic acid, isobutyric acid, valeric acid, isovaleric acid, citric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, crotonic acid, myristoleic acid, palmitoleic acid, sapienic acid, oleic acid, elaidic acid, vaccenic acid, gadoleic, eicosenoic acid, erucic acid, nervonic acid, linoleic acid (e.g., α-linolenic acid, and γ-linolenic acid), eicosadienoic acid, docosadienoic acid, linolenic acid, pinolenic acid, eleostearic acid, mead acid, dihomo-γ-linolenic acid, eicosatrienoic acid, stearidonic acid, arachidonic acid, eicosatetraenoic acid, adrenic acid, bosseopentaenoic acid, eicosapentaenoic acid, ozubondo acid, sardine acid, tetracosanolpentaenoic acid, docosahexaenoic acid, herring acid, formic acid, oxalic acid, glyoxylic acid, glycolic acid, acrylic acid, malonic acid, pyruvic acid, lactic acid, succinic acid, acetoacetic acid, fumaric acid, maleic acid, oxaloacetic acid, malic acid, tartaric acid, glutaric acid, alpha-ketoglutaric acid, adipic acid, aconitic acid, isocitric acid, sorbic acid, pimelic acid, benzoic acid, salicylic acid, phthalic acid, trimesic acid, cinnamic acid, and sebacic acid.

In certain embodiments, the organic acids are fatty acids. In some variations, the organic acid is a short-chain fatty acid (SCFA), a medium-chain fatty acid (MCFA), a long-chain fatty acid (LCFA), or a very long chain fatty acid (VLCFA). In certain variations, the fatty acids may be saturated. In other variations, the fatty acids are unsaturated. In one variation, the organic acid is a mono-unsaturated fatty acid, a di-unsaturated fatty acid, a tri-unsaturated fatty acid, a tetra-unsaturated fatty acid, a penta-unsaturated fatty acid, or a hexa-unsaturated fatty acid. In other variations, the organic acids may be selected from C1, C2, C3, C4, C5, C6, C7, C8, C9, C10, and C18 fatty acids.

The therapeutic compositions may further comprise any combination of the organic acids described above.

Aromatic Compounds

In some embodiments, the compositions described herein (e.g., the therapeutic compositions) may further comprise at one aromatic compound. In some variations, the aromatic compound is a phenyl substituted with at least one hydroxyl group. Thus, in certain embodiments, the composition may further comprise a phenol, resorcinol and monolignol. In other variations, the aromatic compound is polyphenol, such as tannin or tannic acid.

In some variations, the compositions described (e.g., the therapeutic compositions) herein may further comprise at least one aromatic compound such as a flavonoid, a catechin, a lignan. In one variations, the compositions may further comprise anthocyanins, chalcones, dihydrochalcones, dihydroflavonols, flavanols, flavanones, flavones, flavonols and isoflavonoids. In other variations, the compositions may further comprise alkylmethoxyphenols, alkylphenols, curcuminoids, furanocoumarins, hydroxybenzaldehydes, hydroxybenzoketones, hydroxycinnamaldehydes, hydroxycoumarins, hydroxyphenylpropenes, alkoxyphenols (e.g., methoxyphenols), naphtoquinones, phenolic terpenes, tyrosols, hydroxybenzoic acids, hydroxycinnamic acids, hydroxyphenylacetic acids, hydroxyphenylpropanoic acids, hydroxyphenylpentanoic acids, or a stilbene.

The therapeutic compositions may further comprise any combination of the aromatic compounds described above.

Probiotics

In some embodiments, the therapeutic compositions described herein may further comprise probiotic organisms. In some embodiments, the probiotic organism is a probiotic bacterium. In one variation, the probiotic organism is a yeast.

Examples of probiotics may include organisms classified as genera Anaerofilum, Bacteroides, Blautia, Bifidobacterium, Butyrivibrio, Clostridium, Coprococcus, Dialister, Dorea, Fusobacterium, Eubacterium, Ruminococcus, Peptococcus, Peptostreptococcus, Akkermansia, Faecalibacterium, Roseburia, Prevotella, Lachnospira, Lactobacillus, Phascolarctobacterium, Bacillus, Enterococcus, Escherichia, Streptococcus, Saccharomyces, Streptomyces, and family Christensenellaceae.

In some variations, the therapeutic compositions described herein further comprise an organism classified as genera Bacillus, Lactobacillus, Propionebacterium, Pediococcus, Bifidobacterium, Enterococcus, or Saccharomyces, or any combinations thereof.

Examples of probiotics may include Lactobacillus acidophilus, Lactobacillus crispatus, Lactobacillus casei, Lactobacillus rhamnosus, Lactobacillus reuteri, Lactobacillus fermentum, Lactobacillus plantarum, Lactobacillus sporogenes, Lactobacillus bulgaricus, Bifidobacterum lactis, Bifidobacterum animalis, Bifidobacterum bifidum, Bifidobacterum longum, Bifidobacterum adolescentis, Bifidobacterum infantis, Saccharomyces boulardii, Streptococcus thermophilus, Streptococcus salivarius, Akkermansia municiphilia, Christensenella minuta, Clostridium coccoides, Clostridium leptum, Clostridium scindens, Dialister invisus, Eubacterium rectal, Eubacterium eligens, or Faecalibacterium prausnitzii.

In some variations, the therapeutic compositions described herein further comprise Bacillus subtilis, Bacillus subtilis, Bacillus amyloliquifaciens, Bacillus licheniformis, Bacillus coagulans, Lactobacillus salivarius, Propionebacterium freudenrechii, Pediococcus acidilacticii, Bifidobacterium bifidum, Enterococcus faecium, or Saccharomyces uvarum, or any combinations thereof.

In some embodiments, the probiotic organisms may produce any of the organic acids described above, or produce cytotoxic or cytostatic agents. In one variation, the probiotic organism is a bacteriocins. An example a bacteriocin is nisin.

The probiotic organism can be incorporated into the therapeutic compositions described herein as a culture in water or another liquid or semisolid medium in which the probiotic remains viable; or as a freeze-dried powder containing the probiotic organism.

The compositions (including the therapeutic compositions) may further comprise any combination of the probiotics described above.

Other Therapeutic Agents

In some embodiments, the therapeutic compositions described herein may further comprise an antibiotic. In certain embodiments, the antibiotic is present in the composition in less than 1,000 ppm, less than 500 ppm, less than 100 ppm, less than 50 ppm, less than 22 ppm, or less than 11 ppm. In some variations, the antibiotic is bacitracin, bacitracin methylene disalicylate, bacitracin-zinc, virginiamycin, bambermycin, avilamycin, or efrotomycin, or any combinations thereof.

In other embodiments, the therapeutic compositions described herein may further comprise an antifungal agent, an antiviral agent, or an anti-inflammatory agent (e.g. a cytokine, or a hormone), or any combinations thereof.

In certain variations, the therapeutic compositions described herein may further comprise aminoglycosides, cephalosporins, macrolides, penicillins, polypeptide antibiotics, or tetracyclines, or any combinations thereof.

In one variation, the therapeutic compositions described herein may further comprise amikacin, gentamicin, kanamycin, neomycin, streptomycin, tobramycin, cefamandole, cefazolin, cephalexin, cephaloglycin, cephaloridine, cephalothin, cephapirin, cephradine, erythromycin, troleandomycin, penicillin G, amoxicillin, ampicillin, carbenicillin, cloxacillin, dicloxacillin, methicillin, nafcillin, oxacillin, phenethicillin, ticarcillin, bacitracin, colistimethate, colistin, polymyxin B, chlortetracycline, demeclocycline, doxycycline, methacycline, minocycline, tetracycline, oxytetracycline, chloramphenicol, clindamycin, cycloserine, lincomycin, rifampin, spectinomycin, vancomycin, viomycin, or metronidazole, or any combinations thereof.

The therapeutic compositions may further comprise any combination of the other therapeutic agents described above.

In some aspects, the therapeutic compositions described herein may be administered to animals as part of the diet of the animal. For example, the therapeutic compositions described herein may be incorporated with base feed, and fed to animals as part of their regular diets. As used herein, “animal feed” refers to feed suitable for non-human consumption.

In some variations, an animal feed composition comprises:
(a) a base feed; and
(b) at least one C5 carbohydrate, at least one C6 carbohydrate, at least one C5 deoxy sugar, at least one C6 deoxy sugar, at least one C5 amino sugar, at least one C6 amino sugar, at least one C5 sugar alcohol, at least one C6 sugar alcohol, at least one C5 sugar acid, at least one C6 sugar acid, at least one C5 phosphate sugar, at least one C6 phosphate sugar, at least one C5 sulfate sugar, or at least one C6 sulfate sugar, or
   a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or
   any combinations thereof.

In certain variations, an animal feed composition comprises:
(a) a base feed;
(b) at least one C5 carbohydrate, at least one C6 carbohydrate, at least one C5 deoxy sugar, at least one C6 deoxy sugar, at least one C5 amino sugar, at least one C6 amino sugar, at least one C5 sugar alcohol, at least one C6 sugar alcohol, at least one C5 sugar acid, at least one C6 sugar acid, at least one C5 phosphate sugar, at least one C6 phosphate sugar, at least one C5 sulfate sugar, or at least one C6 sulfate sugar, or
   a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or
any combinations thereof; and
(c) a delivery vehicle.

In some variations, the base feed is a nutritionally sufficient diet to sustain growth. Such diets may be well-known in the industry, and the nutritional content of such diets (including, for example, the content of apparent metabolizable energy, protein, fats, vitamins, and minerals) may fall within industry-recognized ranges or values.

One of skill in the art would recognize that the type of base feed combined with the therapeutic composition may also vary depending on the animal. The animal feed composition or animal feed pre-mix may contain base feed and any therapeutic composition described herein. For example, the base feed for monogastrics, such as poultry, may include wheat, corn and/or soybean; and the base feed for a ruminant is typically hay or live grass.

One of skill in the art would also recognize that the type of base feed combined with the therapeutic compositions may also vary depending on the growth stage of the animal, or the target animal product, or a combination thereof. For example, the base feed selected for an animal in the starter phase may be different from that in the grower phase, and the base feed selected for an animal in the grower phase may be different than that selected for an animal in the finisher phase. In another example, the base feed selected for an animal with a target animal product of meat may be different than that for an animal with a target animal product of milk.

Suitable base feed may include, for example, additional ingredients and/or nutrients in any suitable form (including, for example, solid form or liquid form) comprising protein, carbohydrates, and fat, used in the body of an animal to sustain growth, repair processes, vital processes, and/or furnish energy. In some variations, base feed may include biomass, such as grass, grain, or legumes. In other variations, base feed may include hay, stover, straw, silage, wheat, barley, maize, sorghum, rye, oats, triticale, rice, soybeans, peas, seaweed, yeast, molasses, or any combinations thereof. In yet other variations, base feed may include animal products, for example lactose, milk, milk solids, chicken meal, fish meal, bone meal, or blood, or any combinations thereof. In yet other variations, base feed may include oil, for example, plant oil or animal oil. In another variation, base feed may include hay, straw, silage, oils, grains, legumes, bone meal, blood meal, and meat, or any combinations thereof. In still other variations, base feed may include, for example, fodder, corn-soy based diets, or wheat-soy based diets.

Any other suitable compounds may be present in the base feed, including, for example, essential amino acids, salts, minerals, protein, carbohydrates, and/or vitamins.

In some variations, the base feed comprises copper and/or zinc. In other variations, the base feed further comprises an ionophore or other coccidiostat. In other variations, the base feed does not include an ionophore. In certain variations, the base feed composition has less than 1,000 ppm, less than 500 ppm, less than 100 ppm, or less than 50 ppm of an ionophore or other coccidiostat. In some embodiments, the ionophore is monensin, salinomycin, narasin, or lasolocid, or any combinations thereof.

In some variations, the base feed does not include an antiobiotic. Such antibiotics may include bacitracin, bacitracin methylene disalicylate, bacitracin-zinc, virginiamycin, bambermycin, avilamycin, or efrotomycin, or any combinations thereof.

The compositions described herein may be combined with a carrier material to form an animal feed pre-mix.

In some aspects, provided is an animal feed pre-mix comprising:
(a) a carrier material; and
(b) at least one C5 carbohydrate, at least one C6 carbohydrate, at least one C5 deoxy sugar, at least one C6 deoxy sugar, at least one C5 amino sugar, at least one C6 amino sugar, at least one C5 sugar alcohol, at least one C6 sugar alcohol, at least one C5 sugar acid, at least one C6 sugar acid, at least one C5 phosphate sugar, at least one C6 phosphate sugar, at least one C5 sulfate sugar, or at least one C6 sulfate sugar, or
a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or
any combinations thereof.

In certain aspects, the animal feed pre-mix comprises:
(a) a carrier material;
(b) at least one C5 carbohydrate, at least one C6 carbohydrate, at least one C5 deoxy sugar, at least one C6 deoxy sugar, at least one C5 amino sugar, at least one C6 amino sugar, at least one C5 sugar alcohol, at least one C6 sugar alcohol, at least one C5 sugar acid, at least one C6 sugar acid, at least one C5 phosphate sugar, at least one C6 phosphate sugar, at least one C5 sulfate sugar, or at least one C6 sulfate sugar, or
a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or
any combinations thereof; and
(c) a delivery vehicle.

Suitable carrier materials may include, for example, ground rice hulls, ground oat hulls, feed grade silica gel, feed grade fumed silica, corn gluten feed, corn gluten meal, dried distiller's grains, clay, vermiculite, diatamacious earth, or milled corn, or any combinations thereof. In one variation, the carrier material is milled corn. In another variation, the carrier material is ground rice hulls. In yet another variation, the carrier material is ground oat hulls.

In certain variations, a syrup comprising the compositions described herein (including the therapeutic compositions) is combined with a carrier material to produce the animal feed pre-mix. In some variations, the syrup comprises the compositions described herein (including the therapeutic compositions) and water, wherein the syrup has a final solids content of at least 40%, at least 45%, at least 50%, at least 55%, at least 60%, at least 65%, at least 70%, at least 75%, between 40% and 75%, between 50% and 75%, or between 60 and 70% kg dry solids per kg of syrup. In one embodiment, the syrup comprises the compositions described herein (including the therapeutic compositions) and water, wherein the syrup has a final solids content of about 65% kg dry solids per kg of syrup.

The animal feed pre-mix may be in various forms. In one variations, the animal feed pre-mix is in the form of a dry powder. In some variations, the animal feed pre-mix is in the form of a dry, flowable powder.

In other variations, the animal feed pre-mix has a final moisture content of less than 20 wt %, less than 15 wt %, less than 12 wt %, less than 10 wt %, or less than 5 wt %. In one variation, the animal feed pre-mix has a final moisture content of less than 12 wt %, or less than 10 wt %.

In some variations, the compositions described herein (including the therapeutic compositions) are combined with the carrier material to produce a mixture, and the mixture is dried to produce an animal feed pre-mix with the desired moisture content. Any suitable method of drying may be used. For example, in certain embodiments, the compositions described herein (including the therapeutic compositions) are combined with the carrier material to produce a mixture, and the mixture is dried using a rotating drum drier to produce an animal feed pre-mix with the desired moisture content.

The animal feed pre-mix may comprise the compositions described herein (including the therapeutic compositions) at any suitable concentration. In some embodiments, the animal feed pre-mix comprises at least 1 wt %, at least 5 wt %, at least 10 wt %, at least 15 wt %, at least 20 wt %, at least 25 wt %, at least 30 wt %, at least 35 wt %, at least 40 wt %, at least 45 wt %, between 1 to 80 wt %, between 5 to 70 wt %, between 10 to 60 wt %, between 15 to 50 wt %, or between 20 to 50 wt % kg dry composition described herein (including the therapeutic composition) per kg total premix, including moisture.

In some embodiments, the carrier material comprises copper and/or zinc. In certain variations, the carrier material comprises both copper and zinc. In certain variations, the carrier material comprises growth promoting levels of copper and/or zinc. For example, in one variation, the carrier material has (i) between 10 ppm and 500 ppm copper; and/or (ii) between 10 ppm and 5000 ppm zinc.

In certain variations, the carrier material comprises an ionophore or other coccidiostat. In other variations, the carrier material does not comprise an ionophore. In some variations, the carrier material has less than 1,000 ppm, less than 500 ppm, less than 100 ppm, or less than 50 ppm of an ionophore or other coccidiostat. In some embodiments, the ionophore is monensin, salinomycin, narasin, or lasolocid, or any combinations thereof.

In some embodiments, the carrier material does not comprise an antiobiotic. In certain variations, the carrier material has less than 1,000 ppm, less than 500 ppm, less than 100 ppm, less than 50 ppm, less than 22 ppm, or less than 11 ppm of antibiotic. In some embodiments, the antibiotic is bacitracin, bacitracin methylene disalicylate, bacitracin-zinc, virginiamycin, bambermycin, avilamycin, or efrotomycin, or any combinations thereof.

In certain embodiments, the animal feed pre-mixes may be combined with a base feed to form an animal feed composition.

It should be understood that, in some variations, the animal feed compositions and the animal feed pre-mixes have therapeutic effects when fed to animals.

In other aspects, the therapeutic compositions described herein may also be administered to animals as a therapy, for example, to treat a disease or disorder in the animals. For example, the compositions described herein may be formulated as a medicament, and such medicament is administered to the animals as a therapy.

### Methods of Altering Gut Microbiome

The therapeutic compositions described herein may be administered to an animal to selectively alter the composition of organisms in the gut microbiome of the animal. For example, one or more beneficial bacterial taxa may be increased in the gastrointestinal tract, or one or more pathogenic bacterial taxa may be decreased in the gastrointestinal tract, or any combinations of the foregoing may be achieved, by administering the therapeutic compositions as described herein to an animal.

Altering the composition of organisms in the gut microbiome may alter the total production of bacterial metabolites and/or the ratio of bacterial metabolites in the gastrointestinal tract, which may have beneficial effects on animal health. For example, short chain fatty acids are a group of bacterial metabolites, some of which may have beneficial effects on animal health, including reduction in blood serum lipids, increased cardiovascular health, and decreased colon cancer risk. Thus, in certain aspects, provided is also a method of increasing short chain fatty acid production in a gastrointestinal system of an animal, by administering to the animal any of the therapeutic compositions described herein to increase short chain fatty acid production in the animal.

Altering the composition of organisms in the gut microbiome may alter the production of gut peptides by the gastrointestinal tract, which may have beneficial effects on animal health. Gut peptides produced by the gastrointestinal tract may act directly as hormones, or mediate hormone production, and can modulate animal metabolic processes including glycogen synthesis, insulin secretion, and b-cell proliferation in the pancreas.

In certain aspects, provided is a method of altering growth of bacteria in a gastrointestinal system of an animal by administering any of the therapeutic compositions. In some variations, provided is a method of modulating gut microbiome of an animal by administering any of the therapeutic compositions described herein to the animal.

In other aspects, provided is also a method of increasing the diversity of an animal's microbiota, such as increasing the total number of species, creating a more uniform distribution (e.g., increasing the Shannon entropy) over species, or affecting the distribution over taxa and/or phyla (e.g., changing firmicute to bacteroidetes ratio), by administering any of the therapeutic compositions described herein to the animal.

In certain aspects, provided is also a method of selectively modifying growth of bacteria in an animal's gastrointestinal system by administering any of the therapeutic compositions described herein to the animal.

In some variations, the bacteria include Bifidobacteria, lactic acid-producing bacteria (i.e. Lactobacilli), butyrate-producing bacteria, or propionate-producing bacteria; or Clostridia, Bacteroides, or sulfate reducing bacteria (i.e., Desulfovibrio); or Achromobacter spp, Acidaminococcus fermentans, Acinetobacter calcoaceticus, Actinomyces spp, Actinomyces viscosus, Actinomyces naeslundii, Aeromonas spp, Aggregatibacter actinomycetemcomitans, Anaerobiospirillum spp, Alcaligenes faecalis, Arachnia propionica, Bacillus spp, Bacteroides spp, Bacteroides gingivalis, Bacteroides fragilis, Bacteroides intermedius, Bacteroides melaninogenicus, Bacteroides pneumosintes, Bacterionema matruchotii, Corynebacterium matruchotii, Bifidobacterium spp, Buchnera aphidicola, Butyriviberio fibrosolvens, Campylobacter spp, Campylobacter coli, Campylobacter sputorum, Campylobacter upsaliensis, Candida albicans, Capnocytophaga spp, Clostridium spp, Citrobacter freundii, Clostridium difficile, Clostridium sordellii, Corynebacterium spp, Eikenella corrodens, Enterobacter cloacae, Enterococcus spp, Enterococcus faecalis, Enterococcus faecium, Escherichia coli, Eubacterium spp, Flavobacterium spp, Fusobacterium spp, Fusobacterium nucleatum, Gordonia Bacterium spp, Haemophilus parainfluenzae, Haemophilus paraphrophilus, Lactobacillus spp, Leptotrichia buccalis, Methanobrevibacter smithii, Morganella morganii, Mycobacteria spp, Mycoplasma spp, Micrococcus spp, Mycoplasma spp, Mycobacterium chelonae, Neisseria spp, Neisseria sicca, Peptococcus spp, Peptostreptococcus spp, Plesiomonas shigelloides, Porphyromonas gingivalis, Propionibacterium spp, Propionibacterium acnes, Providencia spp, Pseudomonas aeruginosa, Ruminococcus Rothia dentocariosa, Ruminococcus spp, Sarcina spp, Staphylococcus aureus, Staphylococcus epidermidis, Streptococcus anginosus, Streptococcus mutans, Streptococcus oralis, Streptococcus pneumonia, Streptococcus sobrinus, Streptococcus viridans, Torulopsis glabrata, Treponema denticola, Treponema refringens, Veillonella spp, Vibrio spp, Vibrio sputorum, Wolinella succinogenes, or Yersinia enterocolitica, or any combinations of the foregoing.

In other variations, the bacteria are of genera Bacteroides, Odoribacter, Parabacteroides, Alistipes, Blautia, Clostridium, Coprococcus, Dorea, Eubacterium, Lachnospira, Roseburia, Ruminococcus, Faecalibacterium, Oscillospira, Subdoligranulum, Akkermansia, Anaerofilum, Bifidobacterium, Butyrivibrio, Dialister, Fusobacterium, Eubacterium, Lactobacillus, Phascolarctobacterium, Peptococcus, Peptostreptococcus, Prevotella, Roseburia, or Streptococcus.

In certain variations, the bacteria are of species Akkermansia municiphilia, Christensenella minuta, Clostridium coccoides, Clostridium leptum, Clostridium scindens, Dialister invisus, Eubacterium rectal, Eubacterium eligens, Faecalibacterium prausnitzii, Streptococcus salivarius, or Streptococcus thermophilus.

In other variations, the bacteria are of genera Bilophila, Campylobacter, Candidatus, Citrobacter, Clostridium, Collinsella, Desulfovibrio, Enterobacter, Enterococcus, Escherichia, Fusobacterium, Haemophilus, Klebsiella, Lachnospiraceae, Peptostreptococcus, Porphyromonas, Portiera, Providencia, Pseudomonas, Salmonella, Shigella, Staphylococcus, Streptococcus, Vibrio, or Yersinia.

In certain variations, the bacteria are of species Bilophila wadsworthia, Campylobacter jejuni, Citrobacter farmer, Clostridium difficile, Clostridium perfringens, Clostridium tetani, Collinsella aerofaciens, Enterobacter hormaechei, Enterococcus faecalis, Enterococcus faecium, Escherichia coli, Fusobacterium varium, Fusobacterium nucleatum, Haemophilus parainfluenzae, Klebsiella pneumonia, Peptostreptococcus stomatis, Porphyromonas asaccharolytica, Pseudomonas aeruginosa, Salmonella bongori, Salmonella enteric, Shigella boydii, Shigella dysenteriae, Shigella flexneri, Shigella sonnei, Staphylococcus aureus, Streptococcus infantarius, Vibrio cholera, or Yersinia enterocolitica.

In other variations, the bacteria are disease-associated bacteria, pathobionts or pathogens that may be modulated by the therapeutic compositions described herein, and may reside predominantly in one or more specific regions of the gastrointestinal tract.

In certain variations, the bacteria, pathobionts or pathogens may include Listeria, Entamoeba histolytica, Balantidium coli, Basidiobolus ranarum, Trypanosoma cruzi, Clostridium botulinum, Fasciola hepatica, Histoplasma capsulatum, Rotavirus, Schistosoma mansoni, Schistosoma. japonicum, and Schistosoma mekongi, Shigella, Brachyspira aalborgi, Serpulina pilosicoli, Trichuris trichiura, Yersinia enterocolitica, Vibrio, Yersinia enterocolitica, Y. pseudotuberculosis, Clostridium perfringens, CMV virus, Capillaria philippinensis, Cryptosporidium parvum, Cyclospora cayetanensis, Campylobacter, Salmonella, CMV virus, Bacillus anthracis, Candida, Cryptosporidium, EBV (Epstein-Barr virus), Giardia lamblia, H. pylori, H. felis, H. fennelliae, H. cinaedi, Mycobacterium avium, Herpes varicella zoster, Histoplasma, or Toxoplasma.

### Methods of Administration

The therapeutic compositions described herein may be administered to the animal at various doses, on various schedules. In some embodiments, the therapeutic compositions described herein are administered directly to an animal or animal population.

Dose

The therapeutic compositions described herein may be administered to an animal at any appropriate dose to achieve the desired result. One of skill in the art would recognize that the appropriate dose may be different depending on the desired outcome, the type of animal, the age of the animal, and for different breeds of one type of animal.

For example, in some embodiments, the therapeutic compositions described herein are administered at a higher dose to treat a disease or disorder in an animal, and administered at a lower dose to enhance the growth of an animal. In certain variations, the therapeutic compositions are administered at a higher dose to treat a disease or disorder in an animal, and administered at a lower dose to prevent the development of a disease or disorder in the animal.

In some variations, the therapeutic compositions described herein are administered to an animal as a dose of 0.1 mg/g to 20,000 mg/g body weight, or 1 to 500 mg per day.

In some variations, the therapeutic compositions described herein are administered to an animal at a particular inclusion rate. A person of skill in the art would recognize that the inclusion rate of the therapeutic compositions described herein may be different for different types of animal, and may be different for different breeds of one type of animal. The inclusion rate may also be different depending on age of the animal.

In some embodiments, the therapeutic compositions described herein may be provided to an animal at an inclusion rate of less than 0.01 mg/kg, 0.05 mg/kg, 0.1 mg/kg, 1 mg/kg, 10 mg/kg, 50 mg/kg, 100 mg/kg, 200 mg/kg, 300 mg/kg, 400 mg/kg, 500 mg/kg, 600 mg/kg, 700 mg/kg, 800 mg/kg, 900 mg/kg, 1000 mg/kg, 1500 mg/kg, 2000 mg/kg, 2500 mg/kg, 3000 mg/kg, 3500 mg/kg, 4000 mg/kg, 4500 mg/kg, or 5000 mg/kg. In some variations, the therapeutic compositions described herein may be provided to an animal at an inclusion rate of less than 5,000 ppm, less than 4,000 ppm, less than 3,000 ppm, less than 2,000 ppm, less than 2,500 ppm, less than 1,000 ppm, less than 750 ppm, less than 500 ppm, less than 250 ppm, between 10 ppm to 5,000, between 10 ppm and 4,000 ppm, between 10 ppm and 3,000 ppm, between 10 ppm and 2,500 ppm, between 10 ppm and 2,000 ppm, between 10 ppm and 1,000 ppm, between 10 ppm and 500 ppm, between 50 pp and 500 ppm, between 1,000 ppm to 5,000 ppm, between 2,000 ppm to 5,000 ppm, between 3,000 ppm to 5,000 ppm, or between 1,000 ppm to 3,000 ppm.

In some variations, inclusion rate refers to the amount of therapeutic composition included in the total animal feed composition, on a dry weight basis. For example, adding 1 g of dry therapeutic composition to 999 g of dry base feed results in an animal feed composition with a therapeutic composition inclusion rate of 1 g/kg, or 0.1%, or 1000 ppm.

In other variations, the inclusion rate refers to the amount of dry therapeutic composition included in the total animal feed composition, including moisture. For example, adding 1 g of dry therapeutic composition to 999 g of base feed including moisture results in an animal feed composition with a therapeutic composition inclusion rate of 1 g/kg, or 0.1%, or 1000 ppm.

In yet other variations, the inclusion rate refers to the amount of dry therapeutic compositions included in the total animal diet. For example, feeding an animal 1 g of dry compositions directly, wherein the animal also otherwise consumes 999 g of feed in its diet, results in an animal diet with a composition inclusion rate of 1 g/kg, or 0.1%, or 1000 ppm. It should be understood that while inclusion rate may refer to the amount of dry composition included in the total animal diet, the composition may be provided to the animal in any suitable form. For example, in some variations, the composition may be provided to the animal as a dry powder, dry solid, mash, or syrup. In other variations, the composition may be provided to the animal via drinking water. For example, dry composition may be dissolved in drinking water to form a solution with a particular concentration, and the solution provided to the animal.

In certain variations, the inclusion rate refers the amount of dry therapeutic compositions included in a solution provided to the animal (for example, as drinking water). In some variations, the concentration of composition in an aqueous solution (such as drinking water) is between 0.01 to 0.5 grams dry composition per gram aqueous solution, between 0.1 to 0.5 grams dry composition per gram aqueous solution, or between 0.2 to 0.4 grams dry composition per gram aqueous solution.

It should be understood that the dose or inclusion rate may be selected based on the type of animal, the growth stage of the animal, or the animal product produced, or any combinations thereof. For example, the dose or inclusion rate for a ruminant animal may be different than that selected for a monogastric animal. In a second example, the dose or inclusion rate selected for an animal in the grower phase may be different than that selected for an animal in the finisher phase. In yet a third example, the inclusion rate selected for an animal producing meat may be different than that for an animal producing milk.

Schedule of Administration

The therapeutic compositions described herein may be administered to an animal at any appropriate frequency, and over any appropriate time period to achieve the desired result. One of skill in the art would recognize that the appropriate frequency and time period of administration may be different depending on the desired outcome, the type of animal, the age of the animal, and for different breeds of one type of animal.

For example, in some embodiments, therapeutic compositions described herein are administered over a shorter period of time to treat a disease or disorder in an animal, and administered over a longer period of time to enhance the growth of an animal. In certain variations, therapeutic compositions described herein are administered over a shorter period of time to treat a disease or disorder in an animal, and administered over a longer period of time to prevent the development of a disease or disorder in the animal.

In some embodiments, the animal is administered the therapeutic compositions described herein on a daily basis, on a weekly basis, on a monthly basis, on an every other day basis, for at least three days out of every week, or for at least seven days out of every month. In some embodiments, therapeutic compositions described herein are administered to the animal over the entire lifetime of the animal. In some embodiments, the animal is provided therapeutic compositions described herein during certain diet phases.

In certain variations, therapeutic compositions described herein are administered to the animal during the starter diet phase, the grower diet phase, or the finisher diet phase, or any combinations thereof.

In some embodiments, therapeutic compositions described herein are administered to the animal during a treatment period. For example, in certain variations, the treatment period is one day, two days, three days, four days, five days, six days, seven days, one week, two weeks, three weeks, four weeks, five weeks, six weeks, one month, two months, three months, four months, five months, or six months. In certain embodiments, therapeutic compositions described herein are administered to the animal once time, two times, three times, four times, five times, six times, seven times, eight times, nine times, or ten times over the treatment period. In other embodiments, therapeutic compositions described herein are administered to the animal once time, two times, three times, four times, five times, six times, seven times, eight times, nine times, or ten times per day over the treatment period. In yet other embodiments, the therapeutic compositions described herein are administered to the animal once time, two times, three times, four times, five times, six times, seven times, eight times, nine times, or ten times per week over the treatment period.

Modes of Administration

The therapeutic compositions described herein may be administered by any suitable methods, including for example parenteral and enteral techniques. Parenteral administration modalities include those in which the composition is administered by a route other than through the gastrointestinal tract, for example, intravenous, intraarterial, intraperitoneal, intramedullary, intramuscular, intraarticular, intrathecal, and intraventricular injections. Enteral administration modalities include, for example, oral, buccal, sublingual, and rectal administration. For example, in some variations, the therapeutic compositions described herein may be administered orally, intravenously or by inhalation.

The therapeutic compositions described herein may be administered to an animal in any appropriate form, including, for example, in solid form, in liquid form, or a combination thereof. In certain embodiments, the therapeutic compositions described herein may be a liquid, such as a syrup or a solution. In other embodiments, the therapeutic compositions described herein may be a solid, such as pellets or powder. In yet other embodiments, the therapeutic compositions described herein may be administered to the animal in both liquid and solid components, such as in a mash. In one variation, the therapeutic compositions described herein are administered orally in the form of a tablet, pill or capsule.

In certain embodiments, the therapeutic compositions described herein may be administered to an animal separately, and in addition to, other therapeutically active agents, prebiotic substances, and/or probiotic agents. For example, a therapeutically active agent, prebiotic substance, and/or probiotic agent may be administered prior to, concurrent with, or after administration of the therapeutic compositions described herein.

### Articles of Manufacture and Kits

The therapeutic compositions described herein can be prepared and placed in an appropriate container, and labeled for treatment of a disease or disorder. Accordingly, in some aspects, provided is also an article of manufacture, such as a container comprising a unit dosage form of the therapeutic compositions described herein, and a label containing instructions for use of such compositions.

As used herein, “unit dosage form” refers to physically discrete units, suitable as unit dosages, each unit containing a predetermined quantity of active ingredient, or compound which may be in a pharmaceutically acceptable vehicle. One of skill in the art would recognize that the unit dosage form may vary depending on the mode of administration.

Kits also are contemplated. For example, a kit can comprise unit dosage forms of the therapeutic compositions described herein, and a package insert containing instructions for use of the compositions in treatment of a disease or disorder.

In some variations of the foregoing articles of manufacture and kits, the disease or disorder is necrotic enteritis, coccidiosis, nutrient malabsorption syndrome, intestinal barrier breakdown, colisepticemia, yolk sack infection, salmonella infection, or campylobacter infection.

### Growth Enhancement

The compositions described herein (including the animal feed compositions and animal feed pre-mixes) may be fed to animals to enhance growth of the animal. For example, in certain variations, feeding the compositions described herein (including the animal feed compositions and animal feed pre-mixes) to an animal increases the rate of weight gain for an animal, decreases mortality, and/or decreases the feed conversion ratio for an animal. In some embodiments, compositions described herein (including the animal feed compositions and animal feed pre-mixes) are fed to an animal population to decrease mortality and/or decrease variability of the final body weight across the population.

Thus, in some aspects, provided is a method of enhancing growth of an animal by:

providing the compositions described herein (including the animal feed compositions and animal feed pre-mixes) to the animal; and

enhancing growth in the animal.

The compositions described herein (including the animal feed compositions and animal feed pre-mixes) may be fed directly to the animal, be processed into an animal feed pre-mix, or incorporated into an animal feed composition fed to the animal. In some embodiments, the animal fed the compositions described herein (including the animal feed compositions and animal feed pre-mixes) may experience enhanced growth as compared to an animal that is not fed such compositions over the same period of time. In some embodiments, an animal population fed the compositions described herein (including the animal feed compositions and animal feed pre-mixes) may experience enhanced growth as compared to an animal population that is not fed such compositions over the same period of time. Enhanced growth may include, for example, an increase in weight gain, a decrease in the food conversion ratio (FCR), an increase in digestibility of provided feed, an increase in released nutrients from provided feed, or a reduced mortality rate, or any combinations thereof.

In some embodiments, an animal population fed the compositions described herein (including the animal feed compositions and animal feed pre-mixes) may experience enhanced growth as compared to an animal population that is not fed such compositions. Enhanced live growth performance of the animal population may include, for example, an increase in weight gain, a decrease in the food conversion ratio (FCR), an increase in digestibility of provided feed, an increase in released nutrients from provided feed, a reduced mortality rate, or an increase in animal uniformity, or any combinations thereof.

Weight Gain

In some embodiments, a subject animal that is fed the compositions described herein (including the animal feed compositions and animal feed pre-mixes) may experience an increase in weight gain, compared to a control animal that is not fed such compositions. In certain embodiments, both the subject animal and the control animal consume the same quantity of feed on a weight basis, but the subject animal provided the compositions described herein (including the animal feed compositions and animal feed pre-mixes) experiences an increase in weight gain compared to the control animal that is fed a diet that does not include such compositions.

The weight gain of an animal may be determined by any suitable methods known in the art. For example, to determine weight gain of an animal that is subjected to a feeding regimen of the compositions described herein (including the animal feed compositions and animal feed pre-mixes), one of skill in the art can measure the mass of the animal prior to the feeding regimen, measure the mass of the animal after the animal is fed such compositions, and determine the difference between those two measurements.

In some variations, the weight gain may be an average daily weight gain (ADG), an average weekly weight gain (AWG), or a final body weight gain (BWG).

Average Daily Weight Gain

In some variations, providing animals with the compositions described herein (including the animal feed compositions and animal feed pre-mixes) results in an increased average daily weight gain than animals provided feed without such compositions. In some variations, providing an animal population with the compositions described herein (including the animal feed compositions and animal feed pre-mixes) results in an increased average daily weight gain than an animal population provided feed without such compositions.

In one embodiment, the average daily weight gain for animal is the weight gained each day by an individual animal, averaged over a given period of time. In some variations, the average daily weight gain for an animal population is the average daily weight gain for each individual animal, averaged over the population; wherein the average daily weight gain is the weight gained each day by the individual animal, averaged over a given period of time. In yet other variations, the average daily weight gain for an animal population is the total weight gained by the population each day, divided by the number of individual animal in the population, averaged over a given period of time. It should be understood that the daily weight gain or average daily weight gain may be further averaged, for example to provide an average daily weight gain across animal populations.

In certain embodiments, an animal provided the compositions described herein (including the animal feed compositions and animal feed pre-mixes) has an average daily weight gain of at least 1%, at least 2%, at least 3%, at least 4%, at least 5%, at least 6%, at least 8%, at least 9%, at least 10%, at least 11%, at least 12%, between 1 to 10%, between 2 to 8%, or between 3 to 5% greater than the average daily weight gain of animal provided a diet that does not include such compositions.

Average Weekly Weight Gain

In some variations, providing animals with the compositions described herein (including the animal feed compositions and animal feed pre-mixes) results in an increased average weekly weight gain than animals provided feed without such compositions. In some variations, providing animal population with the compositions described herein (including the animal feed compositions and animal feed pre-mixes) results in an increased average weekly weight gain than an animal population provided feed without such compositions.

In one embodiment, the average weekly weight gain for animal is the weight gained each week by an individual animal, averaged over a given period of time. In some variations, the average weekly weight gain for an animal population is the average weekly weight gain for each individual animal, averaged over the population; wherein the average weekly weight gain is the weight gained each week by the individual animal, averaged over a given period of time. In yet other variations, the average weekly weight gain for an animal population is the total weight gained by the population each week, divided by the number of individual animal in the population, averaged over a given period of time. It should be understood that the average weekly weight gain may be further averaged, for example to provide an average weekly weight gain across animal populations.

In certain embodiments, animal provided the compositions described herein (including the animal feed compositions and animal feed pre-mixes), animal feed pre-mix, or animal feed composition has an average weekly weight gain of at least 1%, at least 2%, at least 3%, at least 4%, at least 5%, at least 6%, at least 8%, at least 9%, at least 10%, at least 11%, at least 12%, between 1 to 10%, between 2 to 8%, or between 3 to 5% greater than the average weekly weight gain of animals provided a diet that does not include such compositions.

Final Body Weight Gain

In some variations, providing animals with the compositions described herein (including the animal feed compositions and animal feed pre-mixes) results in an increased final body weight gain than animals provided feed without such compositions. In some variations, providing an animal population with the compositions described herein (including the animal feed compositions and animal feed pre-mixes), animal feed pre-mix, or animal feed composition results in an increased average final body weight gain than an animal population provided feed without such compositions.

In some variations, providing animals or an animal population with the compositions described herein (including the animal feed compositions and animal feed pre-mixes) results in a final body weight gain or average final body weight gain that is closer to the performance target maximum than animals or an animal population that is provided feed without such compositions. The performance target maximum generally refers to the highest practical body weight gain observed for a given breed under ideal growing conditions, ideal animal health, and ideal dietary nutrition.

In one embodiment, the final body weight gain is the quantity of weight an individual animal gains over a period of time. For example, in one embodiment, the total body weight gain is the quantity of weight an individual animal gains from 0 days of age until the final weight taken prior to processing of the animal, or the final weight taken on the day of processing of the animal.

In another embodiment, the average total body weight gain is the quantity of weight an individual animal gains over a period of time, averaged across an animal population. For example, in one embodiment, the average total body weight gain is the quantity of weight an individual animal gains from 0 days of age until the final weight taken prior to processing of the animal, or the final weight taken on the day of processing of the animal, averaged across the animal population. In yet another embodiment, the average total body weight gain is the quantity of weight an animal population gains over a period of time, divided by the number of individual animal in the population. For example, in one embodiment, the average total body weight gain is the quantity of weight an animal population gains from 0 days of age until the final weight taken prior to processing of the animal population, or the final weight taken on the day of processing of the animal, divided by the number of individual animal in the population.

It should be understood that the values for total body weight gain and average total body weight gain can be further averaged. For example, the average total body weight gain for different populations of the same type of animals may be averaged to obtain an average total body weight gain across populations.

In certain embodiments, animal provided the compositions described herein (including the animal feed compositions and animal feed pre-mixes) has a final body weight gain of at least 1%, at least 2%, at least 3%, at least 4%, at least 5%, at least 6%, at least 8%, at least 9%, at least 10%, at least 11%, at least 12%, between 1 to 10%, between 2 to 8%, or between 3 to 5% greater than the final body weight gain of animals provided a diet that does not include such compositions.

In certain embodiments, animals provided the compositions described herein (including the animal feed compositions and animal feed pre-mixes) has an average final body weight gain of at least 1%, at least 2%, at least 3%, at least 4%, at least 5%, at least 6%, at least 8%, at least 9%, at least 10%, at least 11%, at least 12%, between 1 to 10%, between 2 to 8%, or between 3 to 5% greater than the average final body weight gain of animals provided a diet that does not include such compositions.

Yield of Animal Product

In certain variations, providing animals with the compositions described herein (including the animal feed compositions and animal feed pre-mixes) results in an increased yield of animal product, as compared to animals provided feed that does not include such compositions. In some embodiments, the animals provided the compositions described herein (including the animal feed compositions and animal feed pre-mixes) yields at least 1%, at least 2%, at least 3%, at least 4%, at least 5%, at least 6%, at least 7%, at least 8%, at least 9%, at least 10%, between 1 to 10%, between 4 to 10%, between 6 to 10%, or between 2 to 8% more animal product compared to animals provided feed that does not include such compositions. For example, in some embodiments, the animal product is the meat of the animal, and animal provided the compositions described herein (including the animal feed compositions and animal feed pre-mixes) yields a greater quantity of meat compared to animals that are not provided such compositions. In some embodiments, providing an animal population the compositions described herein (including the animal feed compositions and animal feed pre-mixes) results in an increased average yield of animal product, as compared to an animal population provided feed that does not include such compositions. In some variations, the average animal product yield is the quantity of animal product yielded from each individual animal, averaged across the animal population.

In some embodiments, the animal product is the meat of animal (e.g., that may be sold to consumers, processed to produce a food product, or consumed by a human).

In some embodiments, the yield of animal product is the yield obtained from an individual animal. In some embodiments, the average yield of animal product is the yield obtained from each individual animal in an animal population, averaged across the population. In yet another embodiment, the average yield of animal product is the total yield of animal product yielded from an animal population, divided by the number of individual animals in the animal population.

In certain variations, animals or animal populations provided the compositions described herein (including the animal feed compositions and animal feed pre-mixes) has a higher average daily weight gain, higher average weekly weight gain, higher final body weight gain, higher average final body weight gain, or increased average yield of animal product, or any combinations thereof, than animals or animal populations provided a diet that does not include such composition, but which does include one or more antibiotics, one or more ionophores, soluble corn fiber, modified wheat starch, or yeast mannan, or any combinations thereof.

Feed Conversion Ratio

In some variations, animals provided the compositions described herein (including the animal feed compositions and animal feed pre-mixes) has a lower feed conversion ratio compared to animals provided a diet that does not include such compositions. In some variations, feed conversion ratio (FCR) refers to the ratio of feed mass input (for example, consumed by the animal) to the animal output, wherein the animal output is the target animal product.

In some variations, the animal is raised for meat, and the target animal output is body mass. Thus, in some variations, the FCR refers to the ratio of the weight of feed consumed compared to the final body weight of the animal prior to processing. In some variations, the FCR refers to the ratio of the weight of feed consumed compared to the final body weight gain of the animal prior to processing. It should be understood that FCR may be measured for animals or a population of animals over different time periods. For example, in some variations, the FCR is an FCR over the entire lifetime of the animal. In other variations, the FCR is a daily FCR, or a weekly FCR, or a cumulative FCR measured up until a particular moment in time (for example, a particular day).

A person of skill in the art would recognize that the performance target minimum feed conversion ratio (optimal FCR) may also be different depending on the type of animal, breed of animal, the age of the animal, or the sex of the animal. A skilled artisan would recognize that the optimal FCR may be different depending on any combination of these factors.

Performance target minimum generally refers to the lowest feed efficiency observed for a given breed under ideal growing conditions, ideal animal health, and ideal dietary nutrition. It is well known to one skilled in the art, that under common growing conditions, animals may not achieve the performance target minimum FCR. Animals may not achieve its performance target minimum FCR due to a variety of health, nutrition, environmental, and/or community influences. In some embodiments, animals may not achieve its performance target minimum FCR due to disease or environmental pathogenic stress. In other embodiments, animals may not achieve its performance target minimum FCR due to excessive environmental temperature (heat stress), or excessive environmental humidity. In yet other embodiments, animals may not achieve its performance target minimum FCR due to crowding, or other social interaction effects, such as difficulty accessing feed or drinking water.

In some variations, animals provided a diet which does not include the compositions described herein (including the animal feed compositions and animal feed pre-mixes) has an FCR that is at least 1% higher than the performance target minimum, at least 2% higher than the performance target minimum, at least 3% higher than the performance target minimum, at least 4% higher than the performance target minimum, at least 5% higher than the performance target minimum, at least 6% higher than the performance target minimum, at least 7% higher than the performance target minimum, at least 8% higher than the performance target minimum, at least 9% higher than the performance target minimum, or at least 10% higher than the performance target minimum FCR. In certain embodiments, animals provided a diet which does not include the compositions described herein (including the animal feed compositions and animal feed pre-mixes) has an FCR that is 1% to 10% higher than the performance target minimum, 2% to 10% higher than the performance target minimum, or 5% to 10% higher than the performance target minimum.

In some variations, animals provided the compositions described herein (including the animal feed compositions and animal feed pre-mixes) has an FCR that is closer to the performance target minimum compared to animals provided a diet that does not include such compositions. In particular embodiments, the animals provided the compositions described herein (including the animal feed compositions and animal feed pre-mixes) has an FCR that is between 0 to 10% higher than the performance target minimum, between 0 to 5% higher than the performance target minimum, or between 0 to 2% higher than the performance target minimum.

In some variations, animals provided the compositions described herein (including the animal feed compositions and animal feed pre-mixes) has a lower feed conversion ratio compared to animals provided a diet that does not include such compositions. For example, in certain variations, the animals provided a diet comprising the compositions described herein (including the animal feed compositions and animal feed pre-mixes) consumes less food but has the same animal output as compared to animals provided a diet that does not include such compositions. In other variations, the animals provided a diet comprising the compositions described herein (including the animal feed compositions and animal feed pre-mixes) consumes the same amount of food but has a higher animal output as compared to animals provided a diet that does not include such compositions. In yet other variations, the animals provided a diet comprising the compositions described herein (including the animal feed compositions and animal feed pre-mixes) consumes less food and has a higher animal output as compared to animals provided a diet that does not include such compositions.

In some variations, the FCR of animals provided the compositions described herein (including the animal feed compositions and animal feed pre-mixes) is reduced at least 1%, at least 2%, at least 4%, at least 6%, at least 8%, at least 10%, at least 12%, between 1 to 10%, between 4 to 10%, between 1 to 8%, between 4 to 8%, between 1 to 6%, or between 4 to 6% as compared to animals provided a diet that does not include such compositions.

In some variations, an animal population provided the compositions described herein (including the animal feed compositions and animal feed pre-mixes) has a lower FCR compared to an animal population provided a diet that does not include such compositions, wherein the FCR is corrected for mortality in the animal population.

In certain variations, animals provided the compositions described herein (including the animal feed compositions and animal feed pre-mixes) has a lower FCR than animals provided a diet that does not include such compositions, but which does include one or more antibiotics, one or more ionophores, soluble corn fiber, modified wheat starch, or yeast mannan, or any combinations thereof.

It is known to one skilled in the art, that when determining FCR, the FCR may be adjusted for mortality to reduce noise due to small number statistics. Methods for adjusting FCR for mortality are well known to one skilled in the art.

Mortality

In some variations, the mortality of animals or animal populations provided the compositions described herein (including the animal feed compositions and animal feed pre-mixes) may be reduced relative to the mortality rate of animals or animal populations not provided such compositions. The reduction of mortality may include, for example, a decrease in the mortality rate on a per head basis. One of skill in the art would recognize that the mortality rate on a per head basis is determined as the ratio of the number of dead animals to the total number of animals at the start of the performance period. The reduction in mortality may include, for example, a reduction in the mortality rate on a per weight basis. One skilled in the art would recognize that the mortality rate on a per weight basis is determined as the ratio of the total weight of animals lost to mortality to the total weight of live animals plus the total weight of dead animals.

In some embodiments, the mortality rate on a per head basis for animals provided a base feed that does not include such compositions is at least 2%, at least 3%, at least 4%, at least 5%, at least 10%, or at least 20%.

In some embodiments, providing the compositions described herein (including the animal feed compositions and animal feed pre-mixes) to animals or animal populations results in a reduction in mortality rate on a per head basis of between 0 to 90%, between 0 to 80%, between 20 to 70%, between 30 to 60%, between 40 to 60%, or between 45 to 55%, as compared to animals or animal populations that is not provided such compositions.

In certain variations, animals provided the compositions described herein (including the animal feed compositions and animal feed pre-mixes) has a lower mortality rate than animals provided a diet that does not include such compositions, but which does include one or more antibiotics, one or more ionophores, soluble corn fiber, modified wheat starch, or yeast mannan, or any combinations thereof.

Uniformity

In other embodiments, an animal population provided the compositions described herein (including the animal feed compositions and animal feed pre-mixes) has an improved uniformity compared to an animal population that is not provided such compositions. Improving uniformity may include, for example, decreasing the relative variability of final body weight in a population of animals, wherein the relative variability is the standard deviation of final body weight divided by the mean final body weight. In some embodiments, the relative variability in final body weight is reduced by at least 10%, at least 15%, at least 20%, at least 25%, at least 30%, at least 35%, at least 40%, at least 45%, at least 50%, at least 55%, at least 60%, at least 65%, at least 70%, between 10 to 75%, between 20 to 60%, between 25 to 50%, between 25 to 40%, or between 30 to 40% for an animal population provided the compositions described herein (including the animal feed compositions and animal feed pre-mixes) has in improved uniformity compared to an animal population that is not provided such compositions.

In some variations, improving the uniformity of an animal population may increase the efficiency of animal processing, including, for example, mechanical processing to obtain meat from the animal.

In certain variations, an animal population provided the compositions described herein (including the animal feed compositions and animal feed pre-mixes) has greater uniformity than an animal population provided a diet that does not include such composition, but which does include one or more antibiotics, one or more ionophores, soluble corn fiber, modified wheat starch, or yeast mannan, or any combinations thereof.

Fatty Acid Concentration

In some embodiments, animals that is fed the compositions described herein (including the animal feed compositions and animal feed pre-mixes) experiences an increase in the volatile fatty acid (VFA) concentration in the digestive system, compared to animals not fed the such compositions. Volatile fatty acids may include, for example, acetic acid, butyric acid, or valeric acid, or combinations thereof. In some embodiments, animals that are fed the compositions described herein (including the animal feed compositions and animal feed pre-mixes) experience an increase in the VFA concentration in the digestive system, compared to the same animals before being fed the compositions described herein (including the animal feed compositions and animal feed pre-mixes). The VFA concentration may be determined by any appropriate method known in the art (i.e. for example, gas chromatography). In certain embodiments, animals that are fed the compositions described herein (including the animal feed compositions and animal feed pre-mixes) experience an increase in VFA concentration in the digestive system of about 1%, about 5%, about 8%, about 10%, about 15%, about 20%, about 25%, about 30%, about 40%, about 50%, about 60%, about 70%, about 80%, about 90%, or about 100%.

In some embodiments, animals that are fed the compositions described herein (including the animal feed compositions and animal feed pre-mixes) experience an increase in the short chain fatty acid (SCFA) concentration in the digestive system, compared to animals not fed such compositions. In some embodiments, animals that are fed the compositions described herein (including the animal feed compositions and animal feed pre-mixes) experience an increase in the SCFA concentration in the digestive system, compared to the same animals before being fed such compositions.

Short chain fatty acids include acetic, propionic, butyric, iso-butyric, 2-methyl-butyric, valeric, iso-valeric, and lactic acid. The SCFA concentration may be determined by any appropriate method known in the art (i.e. for example, gas chromatography). One of skill in the art would appreciate that short chain fatty acids may exist and/or be determined as their respective conjugate bases (e.g., acetate, propionate, butyrate, iso-butyrate, 2-methyl-butyrate, valerate, iso-valerate, and lactate).

In certain embodiments, animals that are fed the compositions described herein (including the animal feed compositions and animal feed pre-mixes) experience an increase in SCFA concentration in the digestive system of about 1%, about 5%, about 8%, about 10%, about 15%, about 20%, about 25%, about 30%, about 40%, about 50%, about 60%, about 70%, about 80%, about 90%, or about 100%.

In some embodiments, the animals experience an increase in the ileal concentration of SCFA. In other embodiments, the animals experience an increase in the hind gut concentration of SCFA. In some variations, the animals provided the compositions described herein (including the animal feed compositions and animal feed pre-mixes) experience an increase in ileal concentration of SCFA or hind gut concentration of SCFA, or combination thereof, of at least 1%, at least 10%, at least 20%, at least 30%, at least 40%, at least 50%, at least 60%, at least 70%, between 1 to 80%, between 10 to 80%, between 10 to 50%, between 30 to 80%, or between 30 to 50% compared to animals not provided such compositions. In certain variations, the SCFA is butyric acid, propionic acid, acetic acid, valeric acid, isobutyric acid, isovaleric acid, 2-methyl-butyric acid, or lactic acid, or any combinations thereof. In one variation, the SCFA is butyric acid or propionic acid, or a combination thereof.

In some embodiments, animals that are fed the compositions described herein (including the animal feed compositions and animal feed pre-mixes) experience a reduction in the presence of pathogenic or otherwise harmful microorganisms within its digestive system. In some embodiments, the compositions described herein (including the animal feed compositions and animal feed pre-mixes) provide a preferential food source for gut microorganisms that are natural competitors to pathogenic or otherwise harmful microorganisms. In other embodiments, the compositions described herein (including the animal feed compositions and animal feed pre-mixes) bind to the exterior surface (e.g., exterior wall carbohydrate receptors) of pathogenic or otherwise harmful microorganisms, suppressing their ability to colonize the gut, for example by decreasing gut-adherence. In some embodiments, the pathogenic or otherwise harmful microorganisms are enterotoxigenic species or strains. In certain embodiments, the pathogenic or otherwise harmful microorganisms are selected from set including members of Campylobacter spp, Salmonella spp, and Eschericia spp. In one embodiment, the pathogenic or otherwise harmful microorganism is Campylobater jejuni or Campylobacter coli.

In some embodiments, animals that are fed the compositions described herein (including the animal feed compositions and animal feed pre-mixes) may not need to be provided antibiotics, or may require a lower dose of antibiotics, in its diet. In some embodiment, animals that are fed the compositions described herein (including the animal feed compositions and animal feed pre-mixes) but not fed antibiotics may exhibit the same or better feed conversion ratio or feed efficiency than animals that are fed antibiotics but not the compositions described herein (including the animal feed compositions and animal feed pre-mixes).

In certain variations, animals provided the compositions described herein (including the animal feed compositions and animal feed pre-mixes) has a higher digestive system SCFA concentration, hind gut SCFA concentration, or ileal SCFA concentration than animals provided a diet that does not include such compositions, but which does include one or more antibiotics, one or more ionophores, soluble corn fiber, modified wheat starch, or yeast mannan, or any combinations thereof.

In some embodiments, animals that are provided the compositions described herein (including the animal feed compositions and animal feed pre-mixes) has greater access to nutrients in the diet than animals provided a diet that does not include such compositions. Nutrients to which animals provided the compositions described herein (including the animal feed compositions and animal feed pre-mixes) have greater access may include, for example, amino acids, metabolic energy, minerals, or vitamins, or any combinations thereof. For example, in certain embodiments, a diet comprising the compositions described herein (including the animal feed compositions and animal feed pre-mixes) is more digestible to animals than a diet that does not comprise such compositions. Digestibility may be measured by, for example, comparing the amount of undigested nutrient residual in the excreta of the animals relative to the amount of nutrient present in the feed.

### Methods of Producing Therapeutic Compositions

In one aspect, provided herein are methods of producing a therapeutic composition suitable for use to improve animal health. In some variations, the method includes combining feed sugar with a catalyst to form a reaction mixture, and producing an oligosaccharide composition from at least a portion of the reaction mixture. In one variation, the therapeutic composition is the oligosaccharide composition produced. In another variation, the method further comprises combining the oligosaccharide produced with at least one pharmaceutically acceptable vehicle, an organic acid, an aromatic compound, or other therapeutic agents to produce the therapeutic composition.

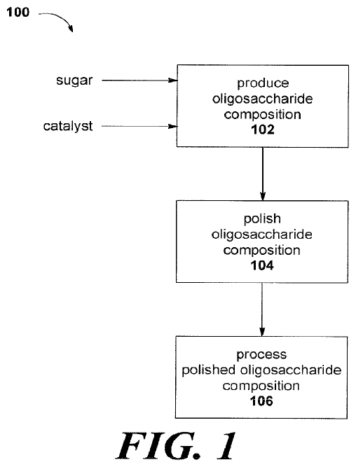

With reference to FIG. 1, process 100 depicts an exemplary process to produce an oligosaccharide composition from sugars, and such oligosaccharide composition produced can subsequently be polished and further processed to form an animal feed ingredient, such as an oligosaccharide syrup or powder. In step 102, one or more sugars are combined with a catalyst in a reactor. The sugars may include, for example, monosaccharides, disaccharides, and/or trisaccharides.

The catalyst has both acidic and ionic groups. In some variations, the catalyst is a polymeric catalyst that includes acidic monomers and ionic monomers. In other variations, the catalyst is a solid-supported catalyst that includes acidic moieties and ionic moieties.

In step 104, the oligosaccharide composition in step 102 is polished to remove fine solids, reduce color, and reduce conductivity, and/or modify the molecular weight distribution. Any suitable methods known in the art to polish the oligosaccharide composition may be used, including, for example, the use of filtration units, carbon or other absorbents, chromatographic separators, or ion exchange columns. For example, in one variation, the oligosaccharide composition is treated with powdered activated carbon to reduce color, microfiltered to remove fine solids, and passed over a strong-acid cationic exchange resin and a weak-base anionic exchange resin to remove salts. In another variation, the oligosaccharide composition is microfiltered to remove fine solids and passed over a weak-base anionic exchange resin. In yet another variation, the oligosaccharide composition is passed through a simulated moving bed chromatographic separator to remove low molecular mass species.

In step 106, the polished oligosaccharide composition undergoes further processing to produce either an oligosaccharide syrup or powder. For example, in one variation, the polished oligosaccharide is concentrated to form a syrup. Any suitable methods known in the art to concentrate a solution may be used, such as the use of a vacuum evaporator. In another variation, the polished oligosaccharide composition is spray dried to form a powder. Any suitable methods known in the art to spray dry a solution to form a powder may be used.

In other variations, process 100 may be modified to have additional steps. For example, the oligosaccharide composition produced in step 102 may be diluted (e.g., in a dilution tank) and then undergo a carbon treatment to decolorize the oligosaccharide composition prior to polishing in step 104. In other variations, the oligosaccharide composition produced in step 102 may undergo further processing in a simulated moving bed (SMB) separation step to reduce digestible carbohydrate content.

In other variations, process 100 may be modified to have fewer steps. For example, in one variation, step 106 to produce the oligosaccharide syrup or powder may be omitted, and the polished oligosaccharide composition of step 104 may be used directly as an ingredient to produce an animal feed composition.

Each of the steps in exemplary process 100, the reactants and processing conditions in each step, as well as the compositions produced in each step are described in further detail below.

a) Feed Sugars

The feed sugar used in the methods of making oligosaccharide compositions described herein may include one or more sugars. In some embodiments, the one or more sugars are selected from monosaccharides, disaccharides, trisaccharides, and short-chain oligosaccharides or any mixtures thereof. In some embodiments, the one or more sugars are monosaccharides, such as one or more C5 or C6 monosaccharides. Exemplary monosaccharides include glucose, galactose, mannose, fructose, xylose, xylulose, and arabinose. In some embodiments, the one or more sugars are C5 monosaccharides. In other embodiments, the one or more sugars are C6 monosaccharides. In some embodiments, the one or more sugars are selected from glucose, galactose, mannose, lactose, or their corresponding sugar alcohols. In other embodiments, the one or more sugars is selected from fructose, xylose, arabinose, or their corresponding sugar alcohols. In some embodiments, the one or more sugars are disaccharides. Exemplary disaccharides include lactose, sucrose and cellobiose. In some embodiments, the one or more sugars are trisaccharides, such as maltotriose or raffinose. In some embodiments, the one or more sugars comprise a mixture of short-chain oligosaccharides, such as maltodextrins. In certain embodiments, the one or more sugars are corn syrup obtained from the partial hydrolysis of corn starch. In a particular embodiment, the one or more sugars is corn syrup with a dextrose equivalent (DE) below 50 (e.g., 10 DE corn syrup, 18 DE corn syrup, 25 DE corn syrup, or 30 DE corn syrup).

In some embodiments, the method includes combining two or more sugars with the catalyst to produce one or more oligosaccharides. In some embodiments, the two or more sugars are selected from glucose, galactose, mannose and lactose (e.g., glucose and galactose).

In other embodiments, the method includes combining a mixture of sugars (e.g., monosaccharides, disaccharides, trisaccharides, etc., and/or other short oligosaccharides) with the catalyst to produce one or more oligosaccharides. In one embodiment, the method includes combining corn glucose syrup with the catalyst to produce one or more oligosaccharides.

In other embodiments, the method includes combining a polysaccharide with the catalyst to produce one or more oligosaccharides. In some embodiments, the polysaccharide is selected from starch, guar gum, xanthan gum and acacia gum.

In other embodiments, the method includes combining a mixture of sugars and sugar alcohols with the catalyst to produce one or more oligosaccharides. In particular embodiments, the method includes combining one or more sugars and one or more alcohols selected from the group consisting of glucitol, sorbitol, xylitol and arabinatol, with the catalyst to produce one or more oligosaccharides.

In some variations of the methods described herein, the sugars may be provided as a feed solution, in which the sugars are combined with water and fed into the reactor. In other variations, the sugars may be fed into the reactor as a solid and combined with water in the reactor.

The sugars used in the methods described herein may be obtained from any commercially known sources, or produced according to any methods known in the art.

b) Catalysts

The catalysts used in the methods of making oligosaccharide compositions described herein include polymeric catalysts and solid-supported catalysts.

In some embodiments, the catalyst is a polymer made up of acidic monomers and ionic monomers (which are also referred to herein as “ionomers”) connected to form a polymeric backbone.

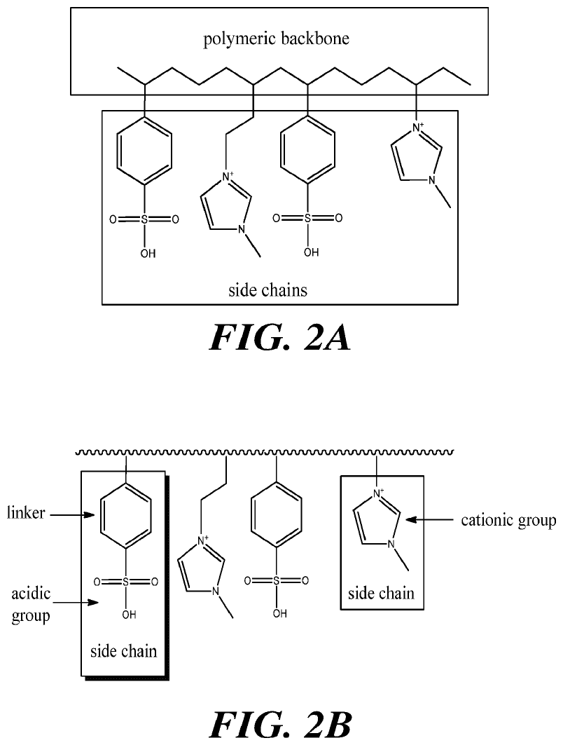

Each acidic monomer includes at least one Bronsted-Lowry acid, and each ionic monomer includes at least one nitrogen-containing cationic group, at least one phosphorous-containing cationic group, or any combination thereof. In certain embodiments of the polymeric catalyst, at least some of the acidic and ionic monomers may independently include a linker connecting the Bronsted-Lowry acid or the cationic group (as applicable) to a portion of the polymeric backbone. For the acidic monomers, the Bronsted-Lowry acid and the linker together form a side chain. Similarly, for the ionic monomers, the cationic group and the linker together form a side chain. With reference to the portion of the polymeric catalyst depicted in FIGS. 2A and 2B, the side chains are pendant from the polymeric backbone.

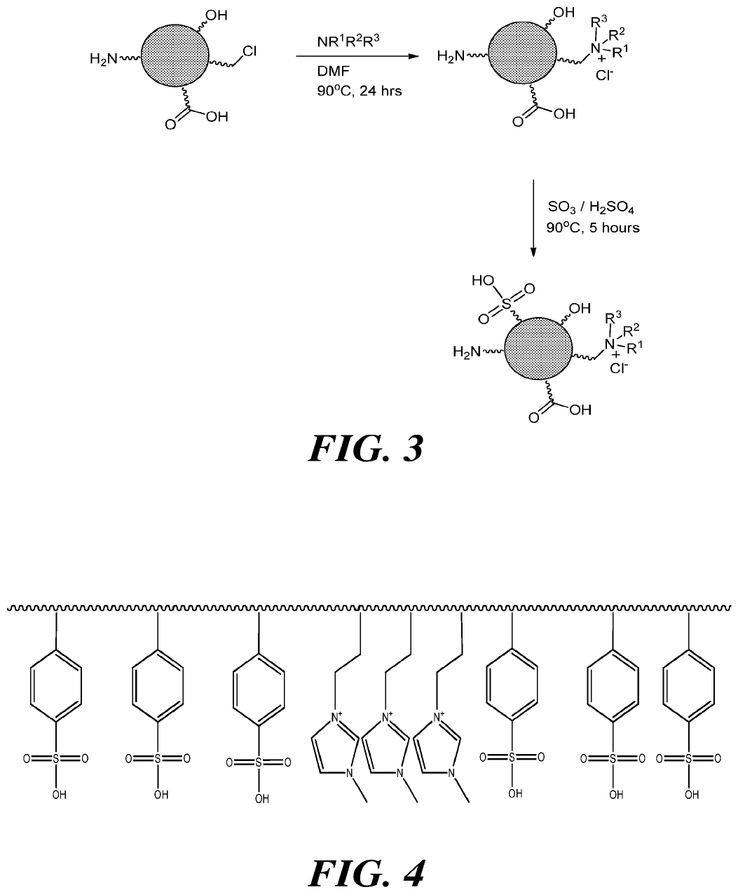

In another aspect, the catalyst is solid-supported, having acidic moieties and ionic moieties each attached to a solid support. Each acidic moiety independently includes at least one Bronsted-Lowry acid, and each ionic moiety includes at least one nitrogen-containing cationic group, at least one phosphorous-containing cationic group, or any combination thereof. In certain embodiments of the solid-supported catalyst, at least some of the acidic and ionic moieties may independently include a linker connecting the Bronsted-Lowry acid or the cationic group (as applicable) to the solid support. With reference to FIG. 3, the produced catalyst is a solid-supported catalyst with acidic and ionic moieties.

Acidic Monomers and Moieties

The polymeric catalysts include a plurality of acidic monomers, where as the solid-supported catalysts include a plurality of acidic moieties attached to a solid support.

In some embodiments, a plurality of acidic monomers (e.g., of a polymeric catalyst) or a plurality of acidic moieties (e.g., of a solid-supported catalyst) has at least one Bronsted-Lowry acid. In certain embodiments, a plurality of acidic monomers (e.g., of a polymeric catalyst) or a plurality of acidic moieties (e.g., of a solid-supported catalyst) has one Bronsted-Lowry acid or two Bronsted-Lowry acids. In certain embodiments, a plurality of the acidic monomers (e.g., of a polymeric catalyst) or a plurality of the acidic moieties (e.g., of a solid-supported catalyst) has one Bronsted-Lowry acid, while others have two Bronsted-Lowry acids.

In some embodiments, each Bronsted-Lowry acid is independently selected from sulfonic acid, phosphonic acid, acetic acid, isophthalic acid, and boronic acid. In certain embodiments, each Bronsted-Lowry acid is independently sulfonic acid or phosphonic acid. In one embodiment, each Bronsted-Lowry acid is sulfonic acid. It should be understood that the Bronsted-Lowry acids in an acidic monomer (e.g., of a polymeric catalyst) or an acidic moiety (e.g., of a solid-supported catalyst) may be the same at each occurrence or different at one or more occurrences.

In some embodiments, one or more of the acidic monomers of a polymeric catalyst are directly connected to the polymeric backbone, or one or more of the acidic moieties of a solid-supported catalyst are directly connected to the solid support. In other embodiments, one or more of the acidic monomers (e.g., of a polymeric catalyst) or one or more acidic moieties (e.g., of a solid-supported catalyst) each independently further includes a linker connecting the Bronsted-Lowry acid to the polymeric backbone or the solid support (as the case may be). In certain embodiments, some of the Bronsted-Lowry acids are directly connected to the polymeric backbone or the solid support (as the case may be), while other the Bronsted-Lowry acids are connected to the polymeric backbone or the solid support (as the case may be) by a linker.

In those embodiments where the Bronsted-Lowry acid is connected to the polymeric backbone or the solid support (as the case may be) by a linker, each linker is independently selected from unsubstituted or substituted alkyl linker, unsubstituted or substituted cycloalkyl linker, unsubstituted or substituted alkenyl linker, unsubstituted or substituted aryl linker, and unsubstituted or substituted heteroaryl linker. In certain embodiments, the linker is unsubstituted or substituted aryl linker, or unsubstituted or substituted heteroaryl linker. In certain embodiments, the linker is unsubstituted or substituted aryl linker. In one embodiment, the linker is a phenyl linker. In another embodiment, the linker is a hydroxyl-substituted phenyl linker.

In other embodiments, each linker in an acidic monomer (e.g., of a polymeric catalyst) or an acidic moiety (e.g., of a solid-supported catalyst) is independently selected from:

unsubstituted alkyl linker;

alkyl linker substituted 1 to 5 substituents independently selected from oxo, hydroxy, halo, amino;

unsubstituted cycloalkyl linker;

cycloalkyl linker substituted 1 to 5 substituents independently selected from oxo, hydroxy, halo, amino;

unsubstituted alkenyl linker;

alkenyl linker substituted 1 to 5 substituents independently selected from oxo, hydroxy, halo, amino;

unsubstituted aryl linker;

aryl linker substituted 1 to 5 substituents independently selected from oxo, hydroxy, halo, amino;

unsubstituted heteroaryl linker; or

heteroaryl linker substituted 1 to 5 substituents independently selected from oxo, hydroxy, halo, amino.

Further, it should be understood that some or all of the acidic monomers (e.g., of a polymeric catalyst) or one or more acidic moieties (e.g., of a solid-supported catalyst) connected to the polymeric backbone by a linker may have the same linker, or independently have different linkers.

In some embodiments, each acidic monomer (e.g., of a polymeric catalyst) and each acidic moiety (e.g., of a solid-supported catalyst) may independently have the structure of Formulas IA-VIA:

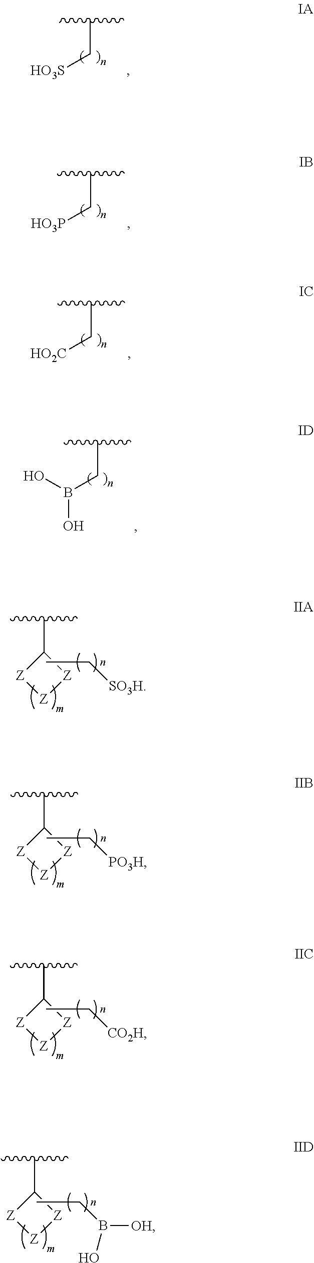

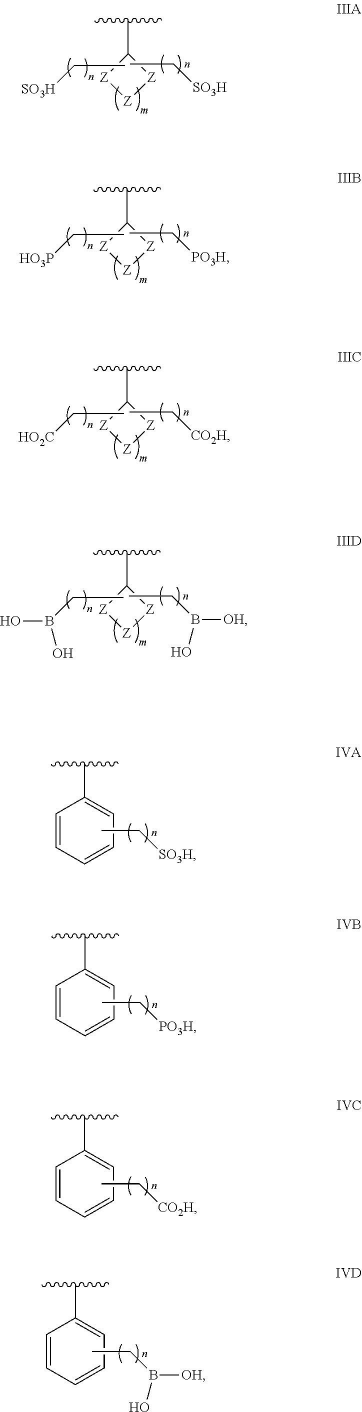

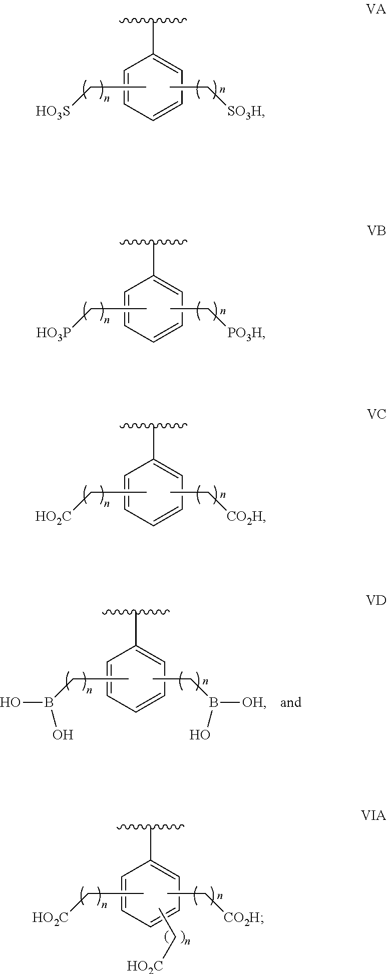

wherein:

each Z is independently C(R²)(R³), N(R⁴), S, S(R⁵)(R⁶), S(O)(R⁵)(R⁶), SO₂, or O, wherein any two adjacent Z can (to the extent chemically feasible) be joined by a double bond, or taken together to form cycloalkyl, heterocycloalkyl, aryl or heteroaryl;

each m is independently selected from 0, 1, 2, and 3;

each n is independently selected from 0, 1, 2, and 3;

each R², R³, and R⁴ is independently hydrogen, alkyl, heteroalkyl, cycloalkyl, heterocyclyl, aryl, or heteroaryl; and

each R⁵ and R⁶ is independently alkyl, heteroalkyl, cycloalkyl, heterocyclyl, aryl, or heteroaryl.

In some embodiments, each acidic monomer (e.g., of a polymeric catalyst) and each acidic moiety (e.g., of a solid-supported catalyst) may independently have the structure of Formulas IA, IB, IVA, or IVB. In other embodiments, each acidic monomer (e.g., of a polymeric catalyst) and each acidic moiety (e.g., of a solid-supported catalyst) may independently have the structure of Formulas IIA, IIB, IIC, IVA, IVB, or IVC. In other embodiments, each acidic monomer (e.g., of a polymeric catalyst) and each acidic moiety (e.g., of a solid-supported catalyst) may independently have the structure of Formulas IIIA, IIIB, or IIIC. In some embodiments, each acidic monomer (e.g., of a polymeric catalyst) and each acidic moiety (e.g., of a solid-supported catalyst) may independently have the structure of Formulas VA, VB, or VC. In some embodiments, each acidic monomer (e.g., of a polymeric catalyst) and each acidic moiety (e.g., of a solid-supported catalyst) may independently have the structure of Formula IA. In other embodiments, each acidic monomer (e.g., of a polymeric catalyst) and each acidic moiety (e.g., of a solid-supported catalyst) may independently have the structure of Formula IB.

In some embodiments, Z can be chosen from C(R₂)(R₃), N(R₄), SO₂, and O. In some embodiments, any two adjacent Z can be taken together to form a group selected from a heterocycloalkyl, aryl, and heteroaryl. In other embodiments, any two adjacent Z can be joined by a double bond. Any combination of these embodiments is also contemplated (as chemically feasible).

In some embodiments, m is 2 or 3. In other embodiments, n is 1, 2, or 3. In some embodiments, R¹ can be hydrogen, alkyl or heteroalkyl. In some embodiments, R¹ can be hydrogen, methyl, or ethyl. In some embodiments, each R², R³, and R⁴ can independently be hydrogen, alkyl, heterocyclyl, aryl, or heteroaryl. In other embodiments, each R², R³ and R⁴ can independently be heteroalkyl, cycloalkyl, heterocyclyl, or heteroaryl. In some embodiments, each R⁵ and R⁶ can independently be alkyl, heterocyclyl, aryl, or heteroaryl. In another embodiment, any two adjacent Z can be taken together to form cycloalkyl, heterocycloalkyl, aryl or heteroaryl.

In some embodiments, the polymeric catalysts and solid-supported catalysts described herein contain monomers or moieties, respectively, that have at least one Bronsted-Lowry acid and at least one cationic group. The Bronsted-Lowry acid and the cationic group can be on different monomers/moieties or on the same monomer/moiety.

In certain embodiments, the acidic monomers of the polymeric catalyst may have a side chain with a Bronsted-Lowry acid that is connected to the polymeric backbone by a linker. In certain embodiments, the acidic moieties of the solid-supported catalyst may have a Bronsted-Lowry acid that is attached to the solid support by a linker. Side chains (e.g., of a polymeric catalyst) or acidic moieties (e.g., of a solid-supported catalyst) with one or more Bronsted-Lowry acids connected by a linker can include, for example,

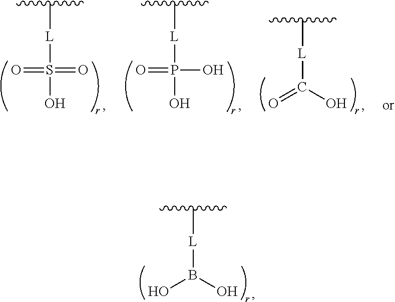

wherein:

L is an unsubstituted alkyl linker, alkyl linker substituted with oxo, unsubstituted cycloalkyl, unsubstituted aryl, unsubstituted heterocycloalkyl, and unsubstituted heteroaryl; and

r is an integer.

In certain embodiments, L is an alkyl linker. In other embodiments L is methyl, ethyl, propyl, butyl. In yet other embodiments, the linker is ethanoyl, propanoyl, benzoyl. In certain embodiments, r is 1, 2, 3, 4, or 5 (as applicable or chemically feasible).

In some embodiments, at least some of the acidic side chains (e.g., of a polymeric catalyst) and at least some of the acidic moieties (e.g., of a solid-supported catalyst) may be:

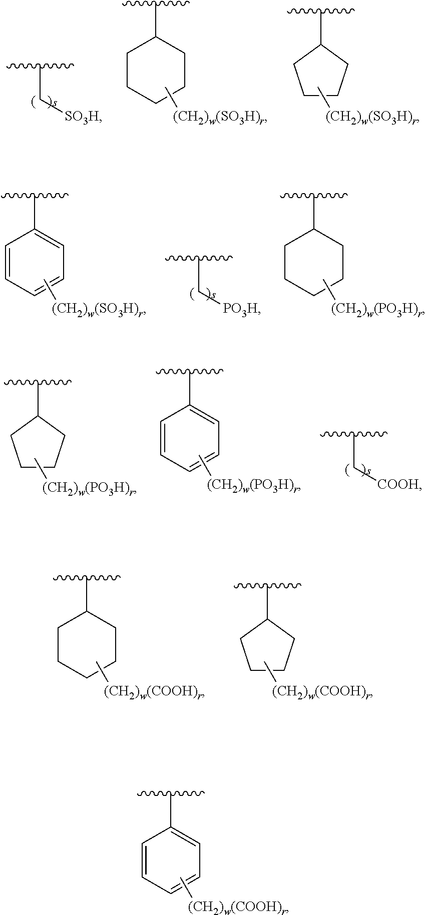

wherein:

s is 1 to 10;

each r is independently 1, 2, 3, 4, or 5 (as applicable or chemically feasible); and

w is 0 to 10.

In certain embodiments, s is 1 to 9, or 1 to 8, or 1 to 7, or 1 to 6, or 1 to 5, or 1 to 4, or 1 to 3, or 2, or 1. In certain embodiments, w is 0 to 9, or 0 to 8, or 0 to 7, or 0 to 6, or 0 to 5, or 0 to 4, or 0 to 3, or 0 to 2, 1 or 0).

In certain embodiments, at least some of the acidic side chains (e.g., of a polymeric catalyst) and at least some of the acidic moieties (e.g., of a solid-supported catalyst) may be:

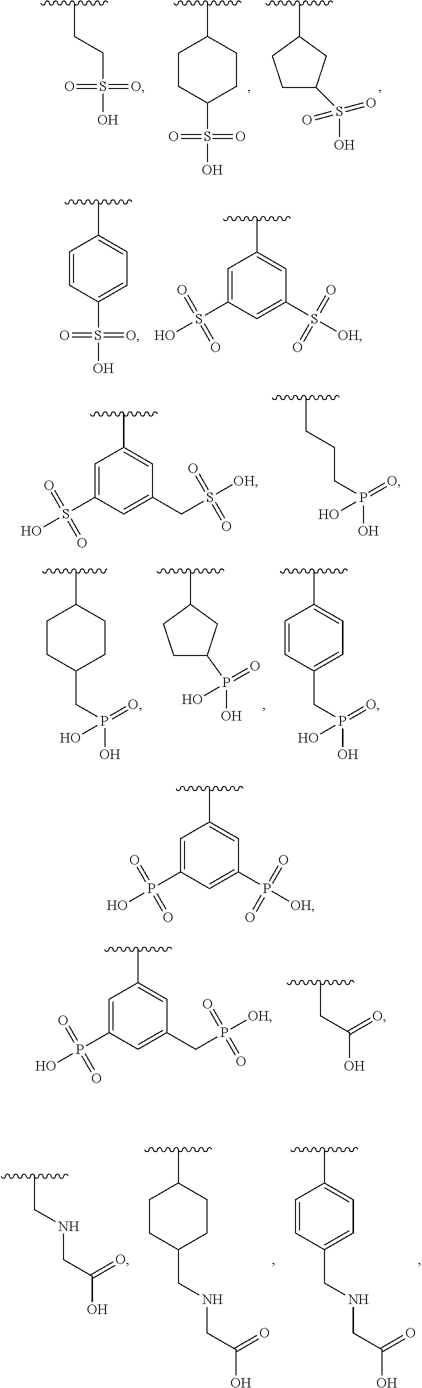

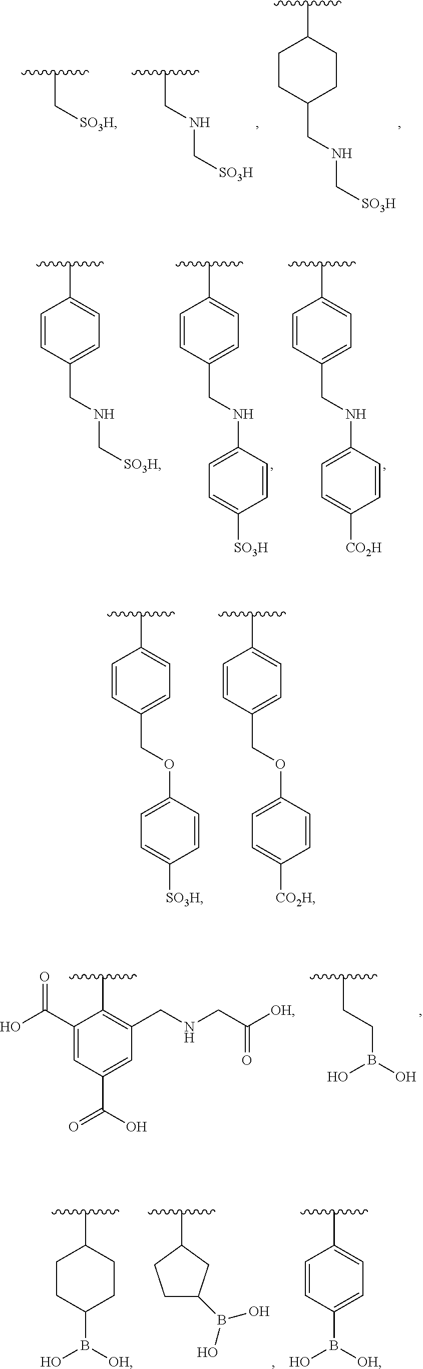

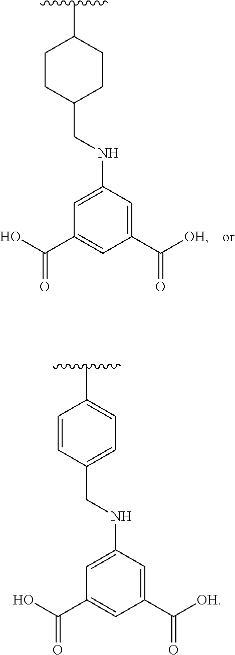 [SMILES: *C1CCC(CNc2cc(C(=O)O)cc(C(=O)O)c2)CC1 ; *c1ccc(CNc2cc(C(=O)O)cc(C(=O)O)c2)cc1]

In other embodiments, the acidic monomers (e.g., of a polymeric catalyst) can have a side chain with a Bronsted-Lowry acid that is directly connected to the polymeric backbone. In other embodiments, the acidic moieties (e.g., of a solid-supported catalyst) may be directly attached to a solid support. Side chains directly connect to the polymeric backbone (e.g., of a polymeric catalyst) or acidic moieties (e.g., of a solid-supported catalyst) directly attached to the solid support may can include, for example,

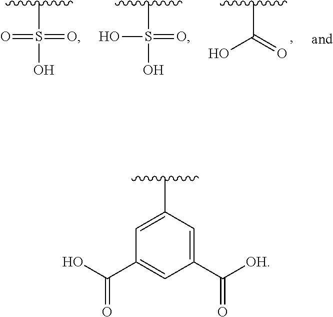

Ionic Monomers and Moieties

The polymeric catalysts include a plurality of ionic monomers, where as the solid-supported catalysts include a plurality of ionic moieties attached to a solid support.

In some embodiments, a plurality of ionic monomers (e.g., of a polymeric catalyst) or a plurality of ionic moieties (e.g., of a solid-supported catalyst) has at least one nitrogen-containing cationic group, at least one phosphorous-containing cationic group, or any combination thereof. In certain embodiments, a plurality of ionic monomers (e.g., of a polymeric catalyst) or a plurality of ionic moieties (e.g., of a solid-supported catalyst) has one nitrogen-containing cationic group or one phosphorous-containing cationic group. In some embodiments, a plurality of ionic monomers (e.g., of a polymeric catalyst) or a plurality of ionic moieties (e.g., of a solid-supported catalyst) has two nitrogen-containing cationic groups, two phosphorous-containing cationic group, or one nitrogen-containing cationic group and one phosphorous-containing cationic group. In other embodiments, a plurality of ionic monomers (e.g., of a polymeric catalyst) or a plurality of ionic moieties (e.g., of a solid-supported catalyst) has one nitrogen-containing cationic group or phosphorous-containing cationic group, while others have two nitrogen-containing cationic groups or phosphorous-containing cationic groups.

In some embodiments, a plurality of ionic monomers (e.g., of a polymeric catalyst) or a plurality of ionic moieties (e.g., of a solid-supported catalyst) can have one cationic group, or two or more cationic groups, as is chemically feasible. When the ionic monomers (e.g., of a polymeric catalyst) or ionic moieties (e.g., of a solid-supported catalyst) have two or more cationic groups, the cationic groups can be the same or different.

In some embodiments, each ionic monomer (e.g., of a polymeric catalyst) or each ionic moiety (e.g., of a solid-supported catalyst) is a nitrogen-containing cationic group. In other embodiments, each ionic monomer (e.g., of a polymeric catalyst) or each ionic moiety (e.g., of a solid-supported catalyst) is a phosphorous-containing cationic group. In yet other embodiments, at least some of ionic monomers (e.g., of a polymeric catalyst) or at least some of the ionic moieties (e.g., of a solid-supported catalyst) are a nitrogen-containing cationic group, whereas the cationic groups in other ionic monomers (e.g., of a polymeric catalyst) or ionic moieties (e.g., of a solid-supported catalyst) are a phosphorous-containing cationic group. In an exemplary embodiment, each cationic group in the polymeric catalyst or solid-supported catalyst is imidazolium. In another exemplary embodiment, the cationic group in some monomers (e.g., of a polymeric catalyst) or moieties (e.g., of a solid-supported catalyst) is imidazolium, while the cationic group in other monomers (e.g., of a polymeric catalyst) or moieties (e.g., of a solid-supported catalyst) is pyridinium. In yet another exemplary embodiment, each cationic group in the polymeric catalyst or solid-supported catalyst is a substituted phosphonium. In yet another exemplary embodiment, the cationic group in some monomers (e.g., of a polymeric catalyst) or moieties (e.g., of a solid-supported catalyst) is triphenyl phosphonium, while the cationic group in other monomers (e.g., of a polymeric catalyst) or moieties (e.g., of a solid-supported catalyst) is imidazolium.

In some embodiments, the nitrogen-containing cationic group at each occurrence can be independently selected from pyrrolium, imidazolium, pyrazolium, oxazolium, thiazolium, pyridinium, pyrimidinium, pyrazinium, pyridazinium, thiazinium, morpholinium, piperidinium, piperizinium, and pyrollizinium. In other embodiments, the nitrogen-containing cationic group at each occurrence can be independently selected from imidazolium, pyridinium, pyrimidinium, morpholinium, piperidinium, and piperizinium. In some embodiments, the nitrogen-containing cationic group can be imidazolium.

In some embodiments, the phosphorous-containing cationic group at each occurrence can be independently selected from triphenyl phosphonium, trimethyl phosphonium, triethyl phosphonium, tripropyl phosphonium, tributyl phosphonium, trichloro phosphonium, and trifluoro phosphonium. In other embodiments, the phosphorous-containing cationic group at each occurrence can be independently selected from triphenyl phosphonium, trimethyl phosphonium, and triethyl phosphonium. In other embodiments, the phosphorous-containing cationic group can be triphenyl phosphonium.

In some embodiments, one or more of the ionic monomers of a polymeric catalyst are directly connected to the polymeric backbone, or one or more of the ionic moieties of a solid-supported catalyst are directly connected to the solid support. In other embodiments, one or more of the ionic monomers (e.g., of a polymeric catalyst) or one or more ionic moieties (e.g., of a solid-supported catalyst) each independently further includes a linker connecting the cationic group to the polymeric backbone or the solid support (as the case may be). In certain embodiments, some of the cationic groups are directly connected to the polymeric backbone or the solid support (as the case may be), while other the cationic groups are connected to the polymeric backbone or the solid support (as the case may be) by a linker.

In those embodiments where the cationic group is connected to the polymeric backbone or the solid support (as the case may be) by a linker, each linker is independently selected from unsubstituted or substituted alkyl linker, unsubstituted or substituted cycloalkyl linker, unsubstituted or substituted alkenyl linker, unsubstituted or substituted aryl linker, and unsubstituted or substituted heteroaryl linker. In certain embodiments, the linker is unsubstituted or substituted aryl linker, or unsubstituted or substituted heteroaryl linker. In certain embodiments, the linker is unsubstituted or substituted aryl linker. In one embodiment, the linker is a phenyl linker. In another embodiment, the linker is a hydroxyl-substituted phenyl linker.

In other embodiments, each linker in an ionic monomer (e.g., of a polymeric catalyst) or an ionic moiety (e.g., of a solid-supported catalyst) is independently selected from:

unsubstituted alkyl linker;

alkyl linker substituted 1 to 5 substituents independently selected from oxo, hydroxy, halo, amino;

unsubstituted cycloalkyl linker;

cycloalkyl linker substituted 1 to 5 substituents independently selected from oxo, hydroxy, halo, amino;

unsubstituted alkenyl linker;

alkenyl linker substituted 1 to 5 substituents independently selected from oxo, hydroxy, halo, amino;

unsubstituted aryl linker;

aryl linker substituted 1 to 5 substituents independently selected from oxo, hydroxy, halo, amino;

unsubstituted heteroaryl linker; or heteroaryl linker substituted 1 to 5 substituents independently selected from oxo, hydroxy, halo, amino.

Further, it should be understood that some or all of the ionic monomers (e.g., of a polymeric catalyst) or one or more ionic moieties (e.g., of a solid-supported catalyst) connected to the polymeric backbone by a linker may have the same linker, or independently have different linkers.

In some embodiments, each ionic monomer (e.g., of a polymeric catalyst) or each ionic moiety (e.g., of a solid-supported catalyst) is independently has the structure of Formulas VIIA-XIB:

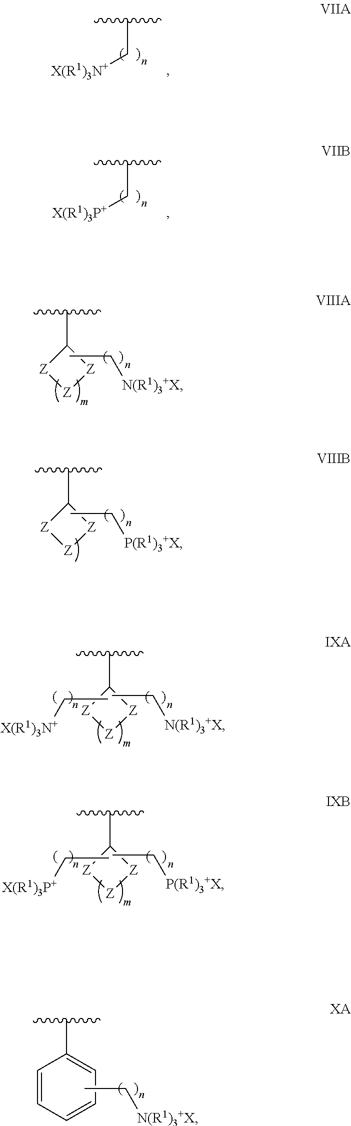

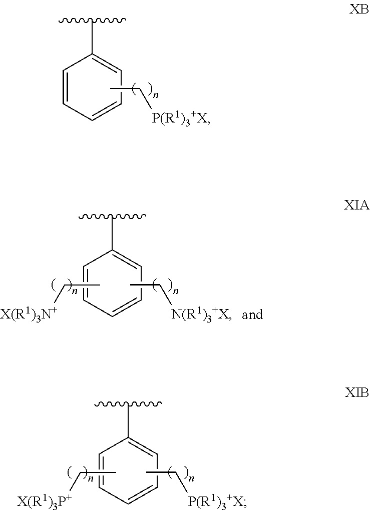

wherein:

each Z is independently C(R²)(R³), N(R⁴), S, S(R⁵)(R⁶), S(O)(R⁵)(R⁶), SO₂, or O, wherein any two adjacent Z can (to the extent chemically feasible) be joined by a double bond, or taken together to form cycloalkyl, heterocycloalkyl, aryl or heteroaryl;

each X is independently F⁻, Cl⁻, Br⁻, I⁻, NO₂⁻, NO₃⁻, SO₄²⁻, R⁷SO₄⁻, R⁷CO₂⁻, PO₄²⁻, R⁷PO₃, or R⁷PO₂⁻, where SO₄²⁻ and PO₄²⁻ are each independently associated with at least two cationic groups at any X position on any ionic monomer, and

each m is independently 0, 1, 2, or 3;

each n is independently 0, 1, 2, or 3;

each R¹, R², R³ and R⁴ is independently hydrogen, alkyl, heteroalkyl, cycloalkyl, heterocyclyl, aryl, or heteroaryl;

each R⁵ and R⁶ is independently alkyl, heteroalkyl, cycloalkyl, heterocyclyl, aryl, or heteroaryl;

and each R⁷ is independently hydrogen, C₁₋₄ alkyl, or C₁₋₄ heteroalkyl.

In some embodiments, Z can be chosen from C(R²)(R³), N(R⁴), SO₂, and O. In some embodiments, any two adjacent Z can be taken together to form a group selected from a heterocycloalkyl, aryl and heteroaryl. In other embodiments, any two adjacent Z can be joined by a double bond. In some embodiments, each X can be Cl⁻, NO₃⁻, SO₄²⁻, R⁷SO₄⁻, or R⁷CO₂⁻, where R⁷ can be hydrogen or C₁₋₄ alkyl. In another embodiment, each X can be Cl⁻, Br⁻, I⁻, HSO₄⁻, HCO₂⁻, CH₃CO₂⁻, or NO₃⁻. In other embodiments, X is acetate. In other embodiments, X is bisulfate. In other embodiments, X is chloride. In other embodiments, X is nitrate.

In some embodiments, m is 2 or 3. In other embodiments, n is 1, 2, or 3. In some embodiments, each R², R³, and R⁴ can be independently hydrogen, alkyl, heterocyclyl, aryl, or heteroaryl. In other embodiments, each R², R³ and R⁴ can be independently heteroalkyl, cycloalkyl, heterocyclyl, or heteroaryl. In some embodiments, each R⁵ and R⁶ can be independently alkyl, heterocyclyl, aryl, or heteroaryl. In another embodiment, any two adjacent Z can be taken together to form cycloalkyl, heterocycloalkyl, aryl or heteroaryl.

In certain embodiments, the ionic monomers of the polymeric catalyst may have a side chain with a cationic group that is connected to the polymeric backbone by a linker. In certain embodiments, the ionic moieties of the solid-supported catalyst may have a cationic group that is attached to the solid support by a linker. Side chains (e.g., of a polymeric catalyst) or ionic moieties (e.g., of a solid-supported catalyst) with one or more cationic groups connected by a linker can include, for example,

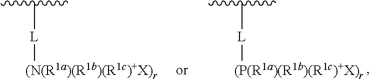

wherein:

L is an unsubstituted alkyl linker, alkyl linker substituted with oxo, unsubstituted cycloalkyl, unsubstituted aryl, unsubstituted heterocycloalkyl, and unsubstituted heteroaryl;

each R^{1a}, R^{1b} and Rio are independently hydrogen or alkyl; or R^{1a} and R^{1b} are taken together with the nitrogen atom to which they are attached to form an unsubstituted heterocycloalkyl; or R^{1a} and R^{1b} are taken together with the nitrogen atom to which they are attached to form an unsubstituted heteroaryl or substituted heteroaryl, and Rio is absent;

r is an integer; and

X is as described above for Formulas VIIA-XIB.

In other embodiments L is methyl, ethyl, propyl, butyl. In yet other embodiments, the linker is ethanoyl, propanoyl, benzoyl. In certain embodiments, r is 1, 2, 3, 4, or 5 (as applicable or chemically feasible).

In other embodiments, each linker is independently selected from:

unsubstituted alkyl linker;

alkyl linker substituted 1 to 5 substituents independently selected from oxo, hydroxy, halo, amino;

unsubstituted cycloalkyl linker;

cycloalkyl linker substituted 1 to 5 substituents independently selected from oxo, hydroxy, halo, amino;

unsubstituted alkenyl linker;

alkenyl linker substituted 1 to 5 substituents independently selected from oxo, hydroxy, halo, amino;

unsubstituted aryl linker;

aryl linker substituted 1 to 5 substituents independently selected from oxo, hydroxy, halo, amino;

unsubstituted heteroaryl linker; or heteroaryl linker substituted 1 to 5 substituents independently selected from oxo, hydroxy, halo, amino.

In certain embodiments, each linker is an unsubstituted alkyl linker or an alkyl linker with an oxo substituent. In one embodiment, each linker is —(CH₂)(CH₂)— or —(CH₂)(C═O). In certain embodiments, r is 1, 2, 3, 4, or 5 (as applicable or chemically feasible).

In some embodiments, at least some of the ionic side chains (e.g., of a polymeric catalyst) and at least some of the ionic moieties (e.g., of a solid-supported catalyst) may be:

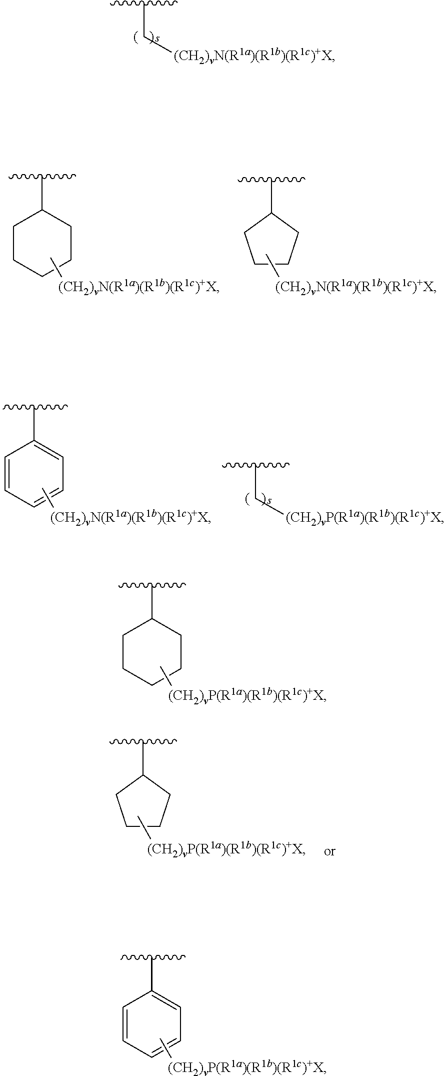

wherein:

each R^{1a}, R^{1b} and R^{1c} are independently hydrogen or alkyl; or R^{1a} and R^{1b} are taken together with the nitrogen atom to which they are attached to form an unsubstituted heterocycloalkyl; or R^{1a} and R^{1b} are taken together with the nitrogen atom to which they are attached to form an unsubstituted heteroaryl or substituted heteroaryl, and R^{1c} is absent;

s is an integer;

v is 0 to 10; and

X is as described above for Formulas VIIA-XIB.

In certain embodiments, s is 1 to 9, or 1 to 8, or 1 to 7, or 1 to 6, or 1 to 5, or 1 to 4, or 1 to 3, or 2, or 1. In certain embodiments, v is 0 to 9, or 0 to 8, or 0 to 7, or 0 to 6, or 0 to 5, or 0 to 4, or 0 to 3, or 0 to 2, 1 or 0).

In certain embodiments, at least some of the ionic side chains (e.g., of a polymeric catalyst) and at least some of the ionic moieties (e.g., of a solid-supported catalyst) may be:

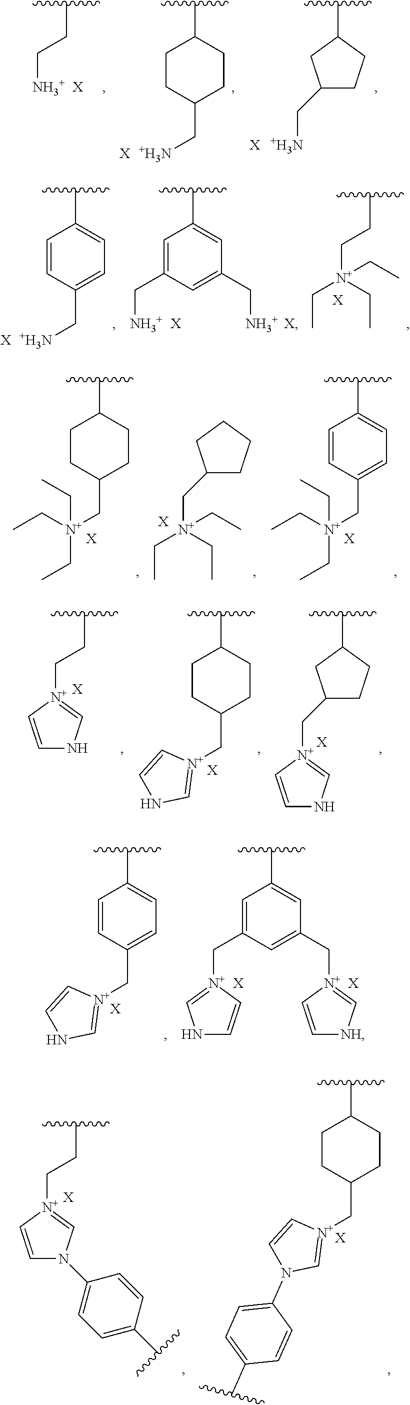

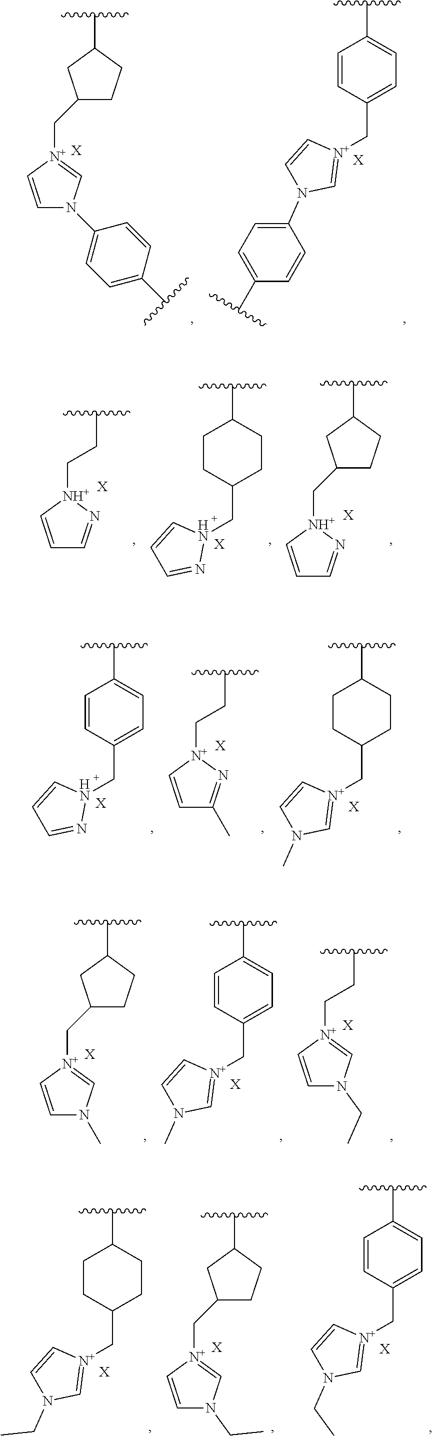

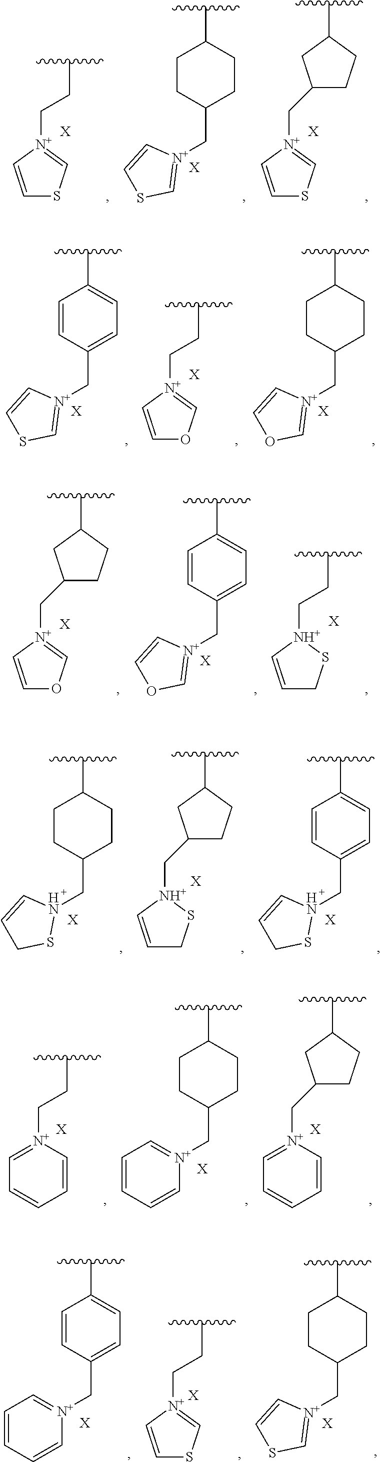

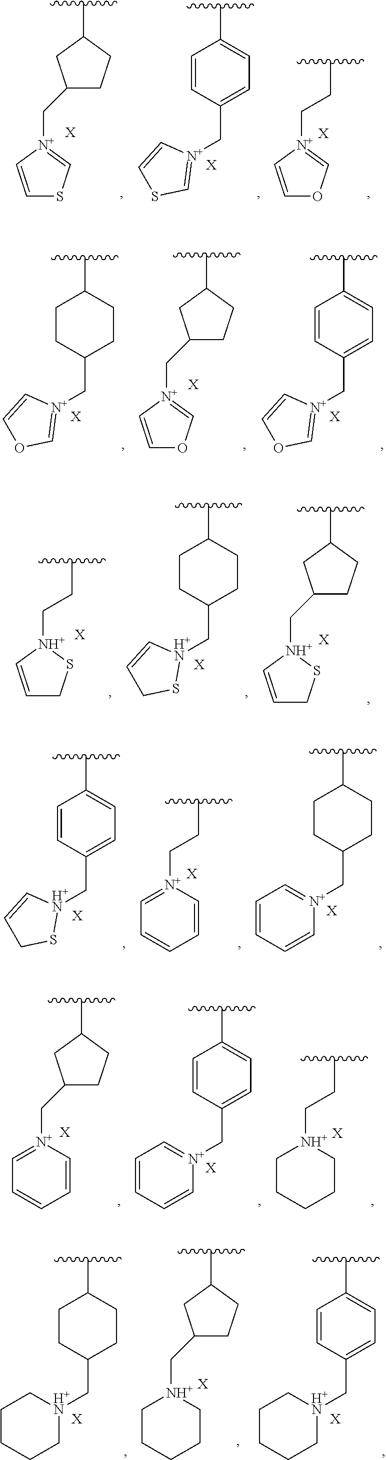

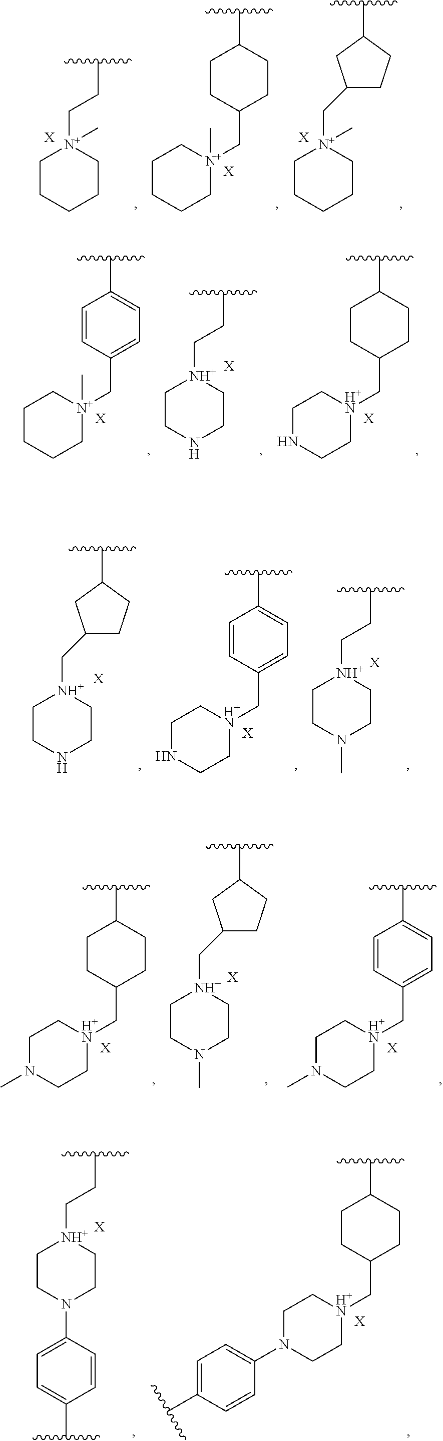

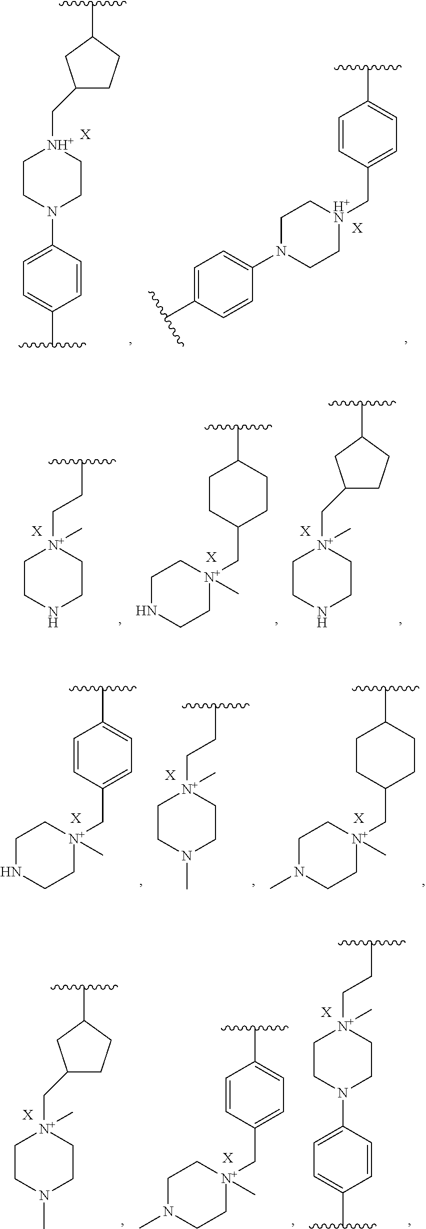

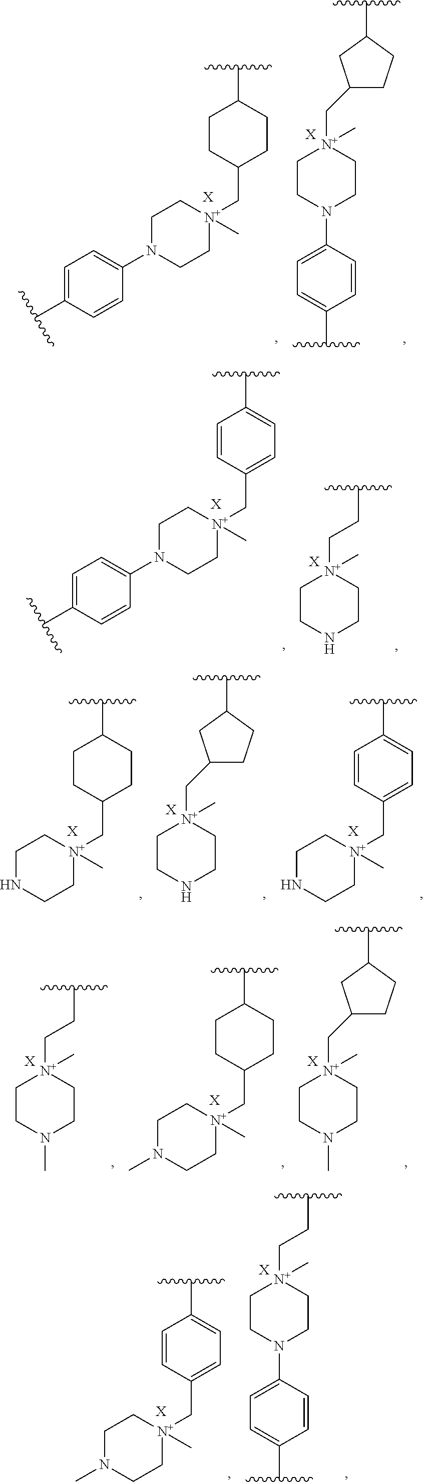

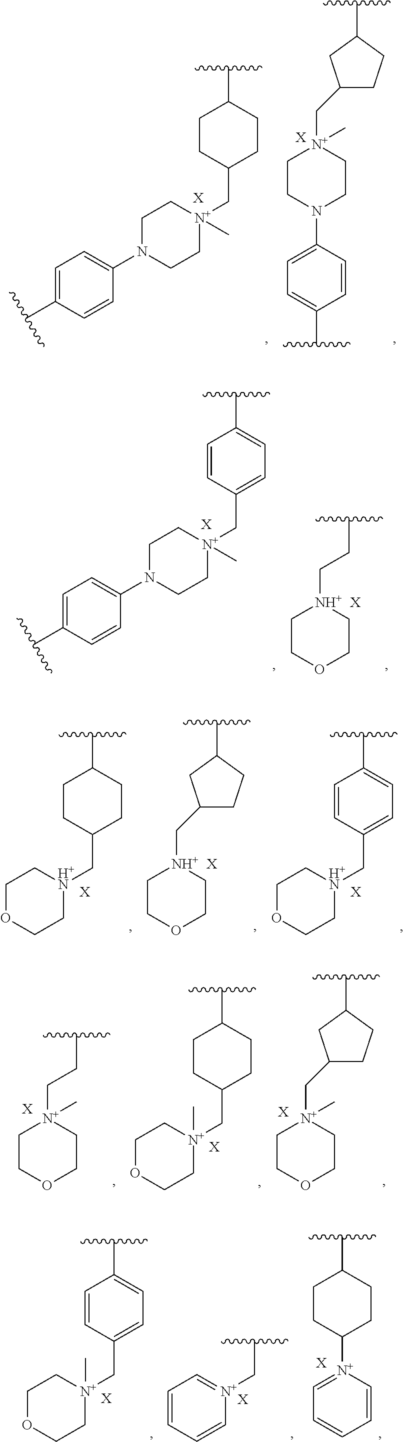

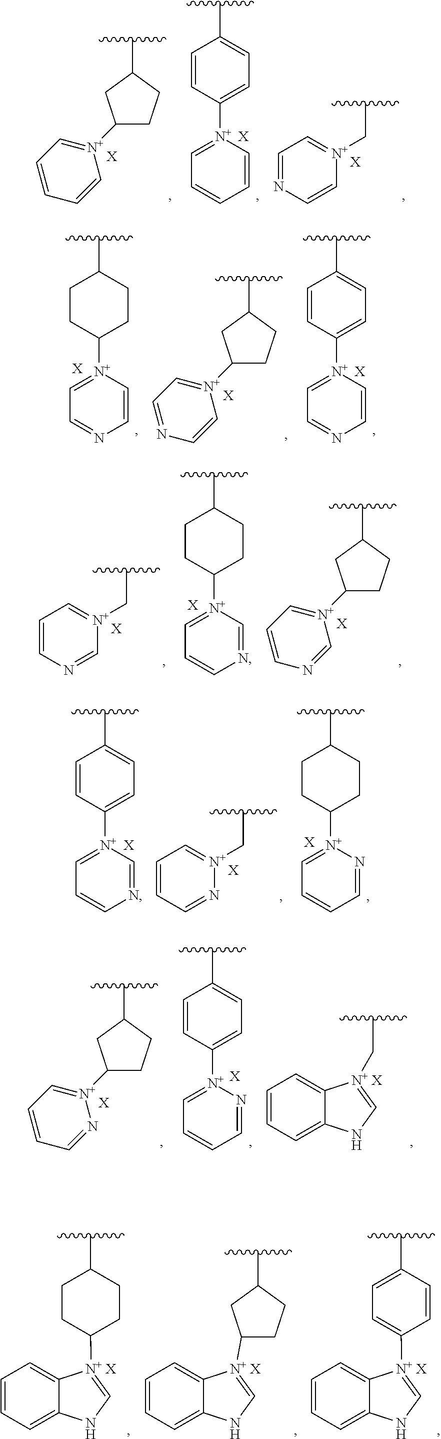

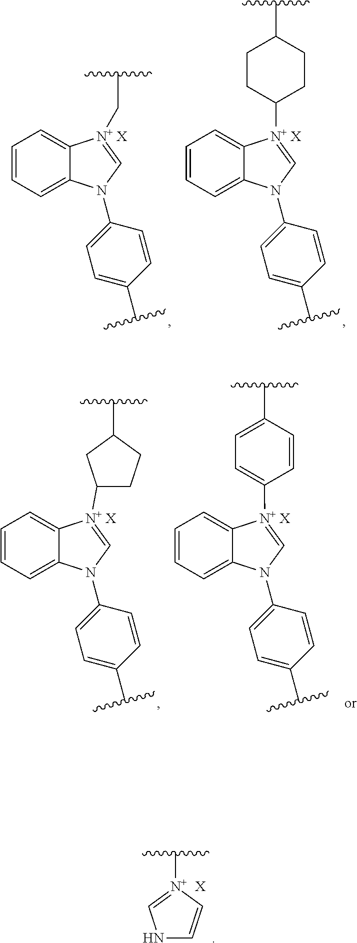

In other embodiments, the ionic monomers (e.g., of a polymeric catalyst) can have a side chain with a cationic group that is directly connected to the polymeric backbone. In other embodiments, the ionic moieties (e.g., of a solid-supported catalyst) can have a cationic group that is directly attached to the solid support. Side chains (e.g., of a polymeric catalyst) directly connect to the polymeric backbone or ionic moieties (e.g., of a solid-supported catalyst) directly attached to the solid support may can include, for example,

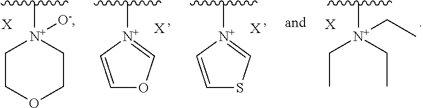

In some embodiments, the nitrogen-containing cationic group can be an N-oxide, where the negatively charged oxide (O—) is not readily dissociable from the nitrogen cation. Non-limiting examples of such groups include, for example,

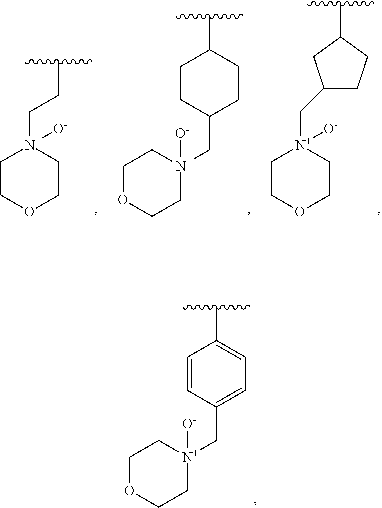 [SMILES: *CC[N+]1([O-])CCOCC1 ; *C1CCC(C[N+]2([O-])CCOCC2)CC1 ; *C1CCC(C[N+]2([O-])CCOCC2)C1 ; *c1ccc(C[N+]2([O-])CCOCC2)cc1]

In some embodiments, the phosphorous-containing side chain (e.g., of a polymeric catalyst) or moiety (e.g., of a solid-supported catalyst) is independently:

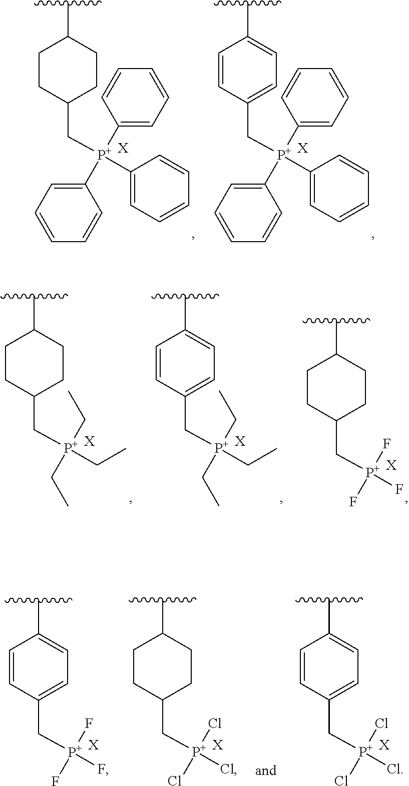

In other embodiments, the ionic monomers (e.g., of a polymeric catalyst) can have a side chain with a cationic group that is directly connected to the polymeric backbone. In other embodiments, the ionic moieties (e.g., of a solid-supported catalyst) can have a cationic group that is directly attached to the solid support. Side chains (e.g., of a polymeric catalyst) directly connect to the polymeric backbone or ionic moieties (e.g., of a solid-supported catalyst) directly attached to the solid support may can include, for example,

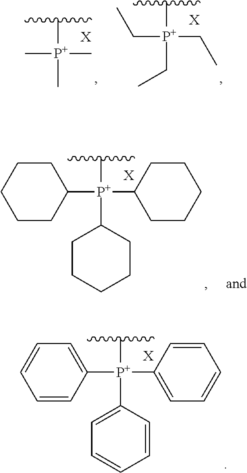

The ionic monomers (e.g., of a polymeric catalyst) or ionic moieties (e.g., of a solid-supported catalyst) can either all have the same cationic group, or can have different cationic groups. In some embodiments, each cationic group in the polymeric catalyst or solid-supported catalyst is a nitrogen-containing cationic group. In other embodiments, each cationic group in the polymeric catalyst or solid-supported catalyst is a phosphorous-containing cationic group. In yet other embodiments, the cationic group in some monomers or moieties of the polymeric catalyst or solid-supported catalyst, respectively, is a nitrogen-containing cationic group, whereas the cationic group in other monomers or moieties of the polymeric catalyst or solid-supported catalyst, respectively, is a phosphorous-containing cationic group. In an exemplary embodiment, each cationic group in the polymeric catalyst or solid-supported catalyst is imidazolium. In another exemplary embodiment, the cationic group in some monomers or moieties of the polymeric catalyst or solid-supported catalyst is imidazolium, while the cationic group in other monomers or moieties of the polymeric catalyst or solid-supported catalyst is pyridinium. In yet another exemplary embodiment, each cationic group in the polymeric catalyst or solid-supported catalyst is a substituted phosphonium. In yet another exemplary embodiment, the cationic group in some monomers or moieties of the polymeric catalyst or solid-supported catalyst is triphenyl phosphonium, while the cationic group in other monomers or moieties of the polymeric catalyst or solid-supported catalyst is imidazolium.

Acidic-Ionic Monomers and Moieties

Some of the monomers in the polymeric catalyst contain both the Bronsted-Lowry acid and the cationic group in the same monomer. Such monomers are referred to as “acidic-ionic monomers”.

Similarly, some of the moieties in the solid-supported catalyst contain both the Bronsted-Lowry acid and the cationic group in the same moieties. Such moieties are referred to as “acidic-ionic moieties”. For example, in exemplary embodiments, the acidic-ionic monomer (e.g., of a polymeric catalyst) or an acidic-ionic moiety (e.g., of a solid-supported catalyst) can contain imidazolium and acetic acid, or pyridinium and boronic acid.

In some embodiments, the monomers (e.g., of a polymeric catalyst) or moieties (e.g., of a solid-supported catalyst) include both Bronsted-Lowry acid(s) and cationic group(s), where either the Bronsted-Lowry acid is connected to the polymeric backbone (e.g., of a polymeric catalyst) or solid support (e.g., of a solid-supported catalyst) by a linker, and/or the cationic group is connected to the polymeric backbone (e.g., of a polymeric catalyst) or is attached to the solid support (e.g., of a solid-supported catalyst) by a linker.

It should be understood that any of the Bronsted-Lowry acids, cationic groups and linkers (if present) suitable for the acidic monomers/moieties and/or ionic monomers/moieties may be used in the acidic-ionic monomers/moieties.

In certain embodiments, the Bronsted-Lowry acid at each occurrence in the acidic-ionic monomer (e.g., of a polymeric catalyst) or the acidic-ionic moiety (e.g., of a solid-supported catalyst) is independently selected from sulfonic acid, phosphonic acid, acetic acid, isophthalic acid, and boronic acid. In certain embodiments, the Bronsted-Lowry acid at each occurrence in the acidic-ionic monomer (e.g., of a polymeric catalyst) or the acidic-ionic moiety (e.g., of a solid-supported catalyst) is independently sulfonic acid or phosphonic acid. In one embodiment, the Bronsted-Lowry acid at each occurrence in the acidic-ionic monomer (e.g., of a polymeric catalyst) or the acidic-ionic moiety (e.g., of a solid-supported catalyst) is sulfonic acid.

In some embodiments, the nitrogen-containing cationic group at each occurrence in the acidic-ionic monomer (e.g., of a polymeric catalyst) or the acidic-ionic moiety (e.g., of a solid-supported catalyst) is independently selected from pyrrolium, imidazolium, pyrazolium, oxazolium, thiazolium, pyridinium, pyrimidinium, pyrazinium, pyridazinium, thiazinium, morpholinium, piperidinium, piperizinium, and pyrollizinium. In one embodiment, the nitrogen-containing cationic group is imidazolium.

In some embodiments, the phosphorous-containing cationic group at each occurrence in the acidic-ionic monomer (e.g., of a polymeric catalyst) or the acidic-ionic moiety (e.g., of a solid-supported catalyst) is independently selected from triphenyl phosphonium, trimethyl phosphonium, triethyl phosphonium, tripropyl phosphonium, tributyl phosphonium, trichloro phosphonium, and trifluoro phosphonium. In one embodiment, the phosphorous-containing cationic group is triphenyl phosphonium.

In some embodiments, the polymeric catalyst or solid-supported catalyst can include at least one acidic-ionic monomer or moiety, respectively, connected to the polymeric backbone or solid support, wherein at least one acidic-ionic monomer or moiety includes at least one Bronsted-Lowry acid and at least one cationic group, and wherein at least one of the acidic-ionic monomers or moieties includes a linker connecting the acidic-ionic monomer to the polymeric backbone or solid support. The cationic group can be a nitrogen-containing cationic group or a phosphorous-containing cationic group as described herein. The linker can also be as described herein for either the acidic or ionic moieties. For example, the linker can be selected from unsubstituted or substituted alkyl linker, unsubstituted or substituted cycloalkyl linker, unsubstituted or substituted alkenyl linker, unsubstituted or substituted aryl linker, and unsubstituted or substituted heteroaryl linker.

In other embodiments, the monomers (e.g., of a polymeric catalyst) or moieties (e.g., of a solid-supported catalyst) can have a side chain containing both a Bronsted-Lowry acid and a cationic group, where the Bronsted-Lowry acid is directly connected to the polymeric backbone or solid support, the cationic group is directly connected to the polymeric backbone or solid support, or both the Bronsted-Lowry acid and the cationic group are directly connected to the polymeric backbone or solid support.

In certain embodiments, the linker is unsubstituted or substituted aryl linker, or unsubstituted or substituted heteroaryl linker. In certain embodiments, the linker is unsubstituted or substituted aryl linker. In one embodiment, the linker is a phenyl linker. In another embodiment, the linker is a hydroxyl-substituted phenyl linker.

Monomers of a polymeric catalyst that have side chains containing both a Bronsted-Lowry acid and a cationic group can also be called “acidic ionomers”. Acidic-ionic side chains (e.g., of a polymeric catalyst) or acidic-ionic moieties (e.g., of a solid-supported catalyst) that are connected by a linker can include, for example,

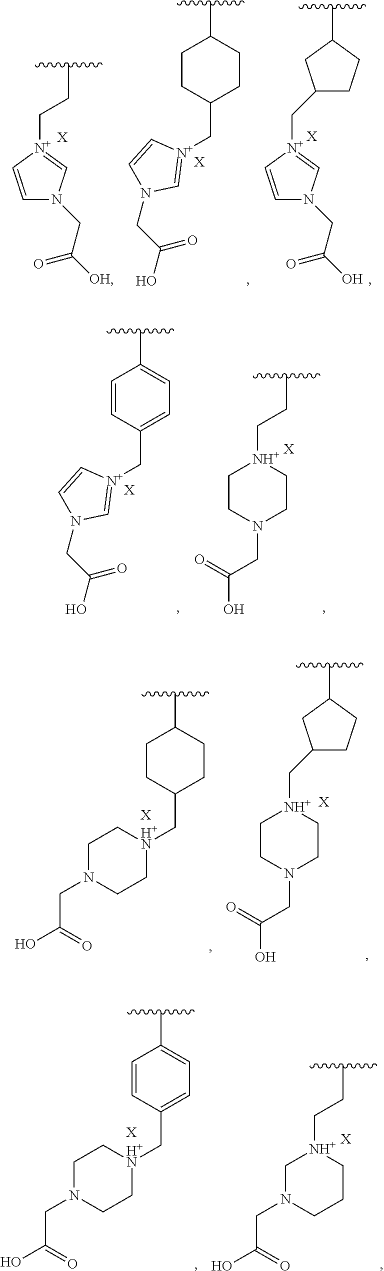

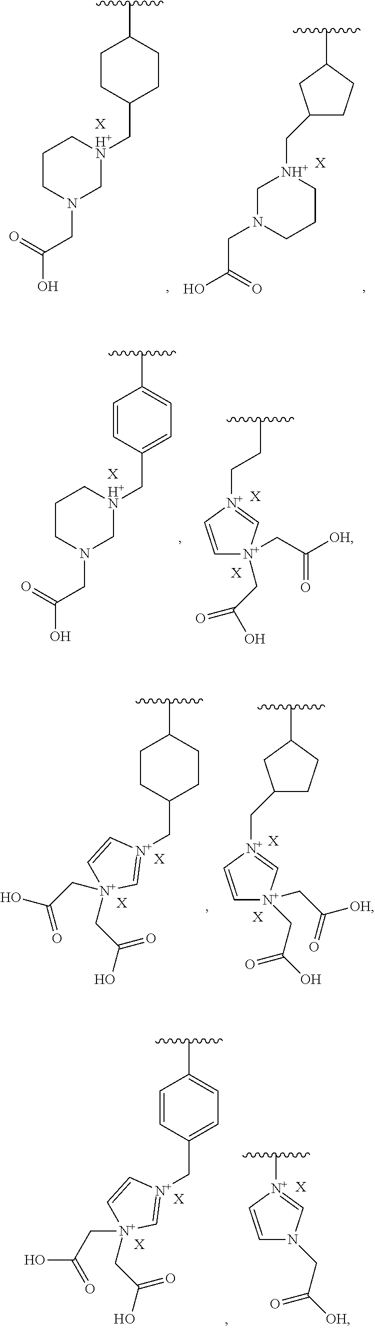

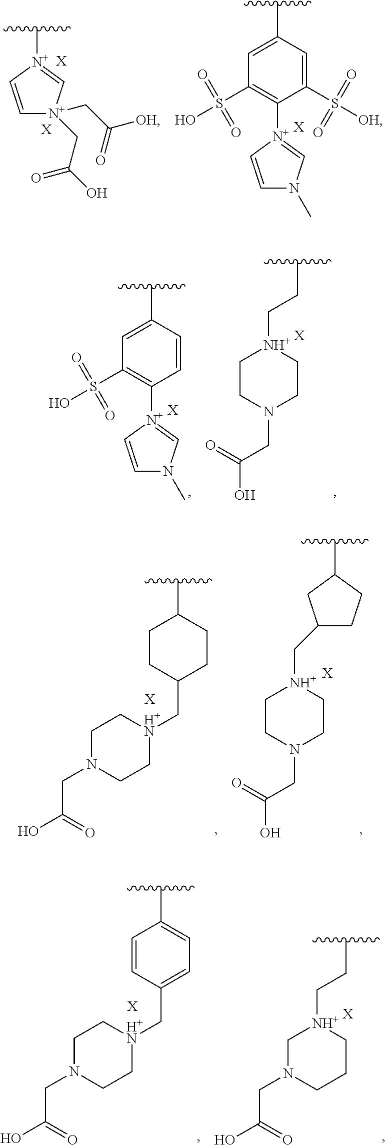

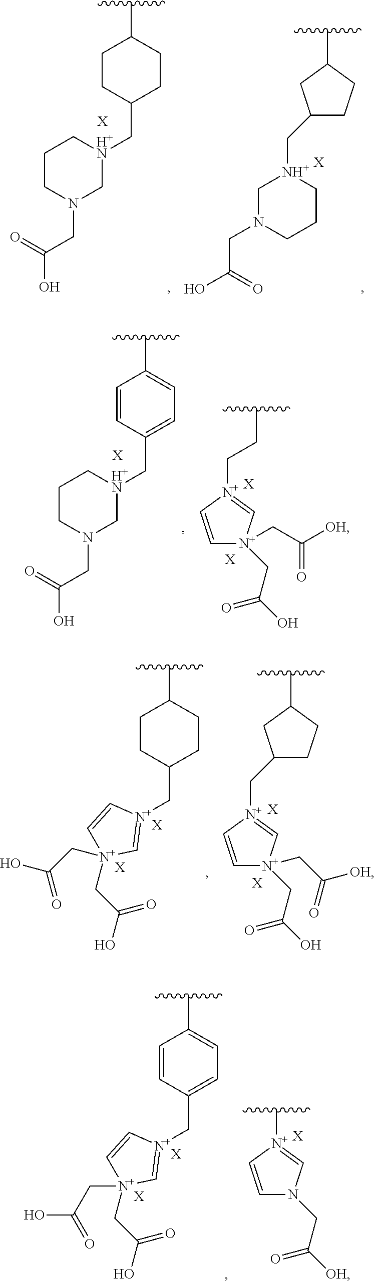

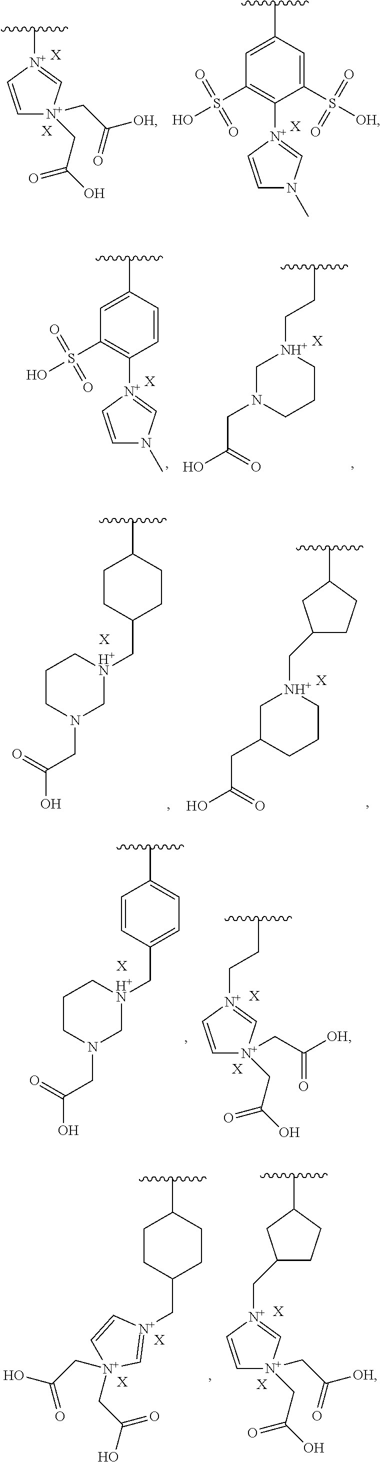

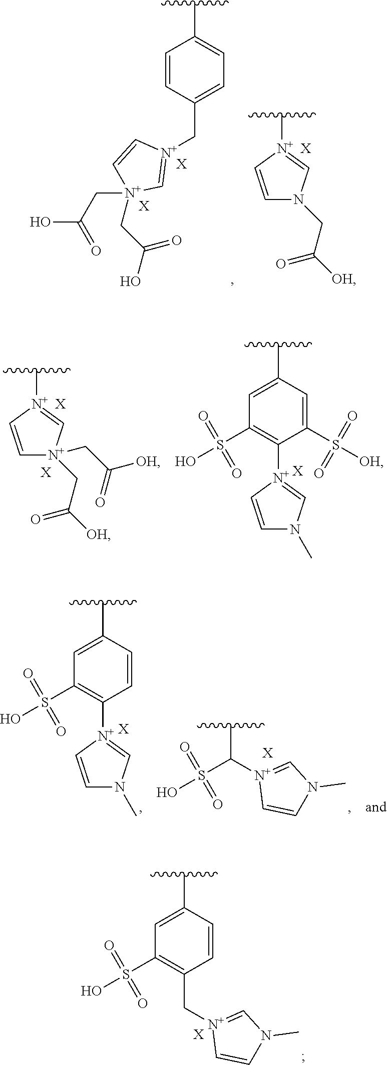

wherein:

each X is independently selected from F⁻, Cl⁻, Br⁻, I⁻, NO₂⁻, NO₃⁻, SO₄²⁻, R⁷SO₄⁻, R⁷CO₂⁻, PO₄²⁻, R⁷PO₃⁻, and R⁷PO₂⁻, where SO₄²⁻ and PO₄²⁻ are each independently associated with at least two Bronsted-Lowry acids at any X position on any side chain, and

each R⁷ is independently selected from hydrogen, C₁₋₄alkyl, and C₁₋₄heteroalkyl.

In some embodiments, R¹ can be selected from hydrogen, alkyl, and heteroalkyl. In some embodiments, R¹ can be selected from hydrogen, methyl, or ethyl. In some embodiments, each X can be selected from Cl⁻, NO₃⁻, SO₄²⁻, R⁷SO₄⁻, and R⁷CO₂⁻, where R⁷ can be selected from hydrogen and C₁₋₄ alkyl. In another embodiment, each X can be selected from Cl⁻, Br⁻, I⁻, HSO₄⁻, HCO₂⁻, CH₃CO₂⁻, and NO₃. In other embodiments, X is acetate. In other embodiments, X is bisulfate. In other embodiments, X is chloride. In other embodiments, X is nitrate.

In some embodiments, the acidic-ionic side chain (e.g., of a polymeric catalyst) or the acidic-ionic moiety (e.g., of a solid-supported catalyst) is independently:

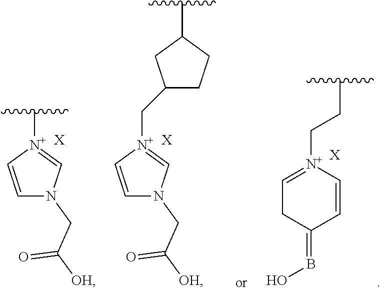

In some embodiments, the acidic-ionic side chain (e.g., of a polymeric catalyst) or the acidic-ionic moiety (e.g., of a solid-supported catalyst) is independently:

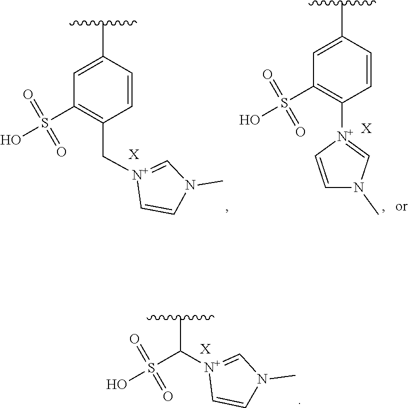

In other embodiments, the monomers (e.g., of a polymeric catalyst) or moieties (e.g., of a solid-supported catalyst) can have both a Bronsted-Lowry acid and a cationic group, where the Bronsted-Lowry acid is directly connected to the polymeric backbone or solid support, the cationic group is directly connected to the polymeric backbone or solid support, or both the Bronsted-Lowry acid and the cationic group are directly connected to the polymeric backbone or solid support. Such side chains in acidic-ionic monomers (e.g., of a polymeric catalyst) or moieties (e.g., of a solid-supported catalyst) can include, for example,

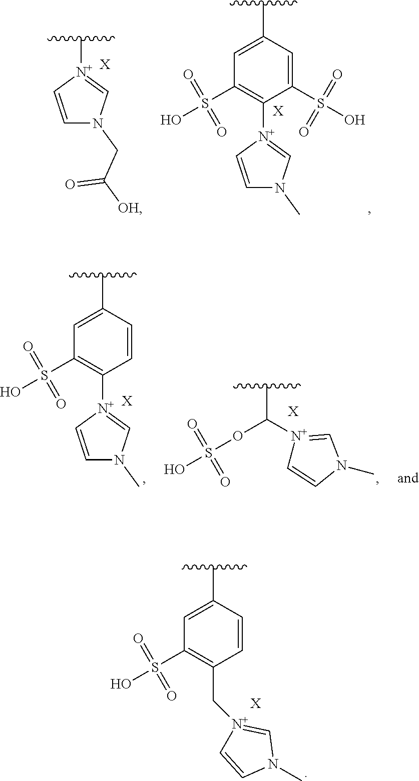

Hydrophobic Monomers and Moieties

In some embodiments, the polymeric catalyst further includes hydrophobic monomers connected to form the polymeric backbone. Similarly, in some embodiments, the solid-supported catalyst further includes hydrophobic moieties attached to the solid support. In either instance, each hydrophobic monomer or moiety has at least one hydrophobic group. In certain embodiments of the polymeric catalyst or solid-supported catalyst, each hydrophobic monomer or moiety, respectively, has one hydrophobic group. In certain embodiments of the polymeric catalyst or solid-supported catalyst, each hydrophobic monomer or moiety has two hydrophobic groups. In other embodiments of the polymeric catalyst or solid-supported catalyst, some of the hydrophobic monomers or moieties have one hydrophobic group, while others have two hydrophobic groups.

In some embodiments of the polymeric catalyst or solid-supported catalyst, each hydrophobic group is independently selected from an unsubstituted or substituted alkyl, an unsubstituted or substituted cycloalkyl, an unsubstituted or substituted aryl, and an unsubstituted or substituted heteroaryl. In certain embodiments of the polymeric catalyst or solid-supported catalyst, each hydrophobic group is an unsubstituted or substituted aryl, or an unsubstituted or substituted heteroaryl. In one embodiment, each hydrophobic group is phenyl. Further, it should be understood that the hydrophobic monomers may either all have the same hydrophobic group, or may have different hydrophobic groups.

In some embodiments of the polymeric catalyst, the hydrophobic group is directly connected to form the polymeric backbone. In some embodiments of the solid-supported catalyst, the hydrophobic group is directly attached to the solid support.

Other Characteristics of the Catalysts

In some embodiments, the acidic and ionic monomers make up a substantial portion of the polymeric catalyst. In some embodiments, the acidic and ionic moieties make up a substantial portion solid-supported catalyst. In certain embodiments, the acidic and ionic monomers or moieties make up at least about 30%, at least about 40%, at least about 50%, at least about 60%, at least about 70%, at least about 80%, at least about 90%, at least about 95%, or at least about 99% of the monomers or moieties of the catalyst, based on the ratio of the number of acidic and ionic monomers/moieties to the total number of monomers/moieties present in the catalyst.

In some embodiments, the polymeric catalyst or solid-supported catalyst has a total amount of Bronsted-Lowry acid of between about 0.1 and about 20 mmol, between about 0.1 and about 15 mmol, between about 0.01 and about 12 mmol, between about 0.05 and about 10 mmol, between about 1 and about 8 mmol, between about 2 and about 7 mmol, between about 3 and about 6 mmol, between about 1 and about 5, or between about 3 and about 5 mmol per gram of the polymeric catalyst or solid-supported catalyst.

In some embodiments of the polymeric catalyst or solid-supported catalyst, each ionic monomer further includes a counterion for each nitrogen-containing cationic group or phosphorous-containing cationic group. In certain embodiments of the polymeric catalyst or solid-supported catalyst, each counterion is independently selected from halide, nitrate, sulfate, formate, acetate, or organosulfonate. In some embodiments of the polymeric catalyst or solid-supported catalyst, the counterion is fluoride, chloride, bromide, or iodide. In one embodiment of the polymeric catalyst or solid-supported catalyst, the counterion is chloride. In another embodiment of the polymeric catalyst or solid-supported catalyst, the counterion is sulfate. In yet another embodiment of the polymeric catalyst or solid-supported catalyst, the counterion is acetate.

In some embodiments, the polymeric catalyst or solid-supported catalyst has a total amount of nitrogen-containing cationic groups and counterions or a total amount of phosphorous-containing cationic groups and counterions of between about 0.01 and about 10 mmol, between about 0.05 and about 10 mmol, between about 1 and about 8 mmol, between about 2 and about 6 mmol, or between about 3 and about 5 mmol per gram of the polymeric catalyst or solid-supported catalyst.

In some embodiments, the acidic and ionic monomers make up a substantial portion of the polymeric catalyst or solid-supported catalyst. In certain embodiments, the acidic and ionic monomers or moieties make up at least about 30%, at least about 40%, at least about 50%, at least about 60%, at least about 70%, at least about 80%, at least about 90%, at least about 95%, or at least about 99% of the monomers of the polymeric catalyst or solid-supported catalyst, based on the ratio of the number of acidic and ionic monomers or moieties to the total number of monomers or moieties present in the polymeric catalyst or solid-supported catalyst.

The ratio of the total number of acidic monomers or moieties to the total number of ionic monomers or moieties can be varied to tune the strength of the catalyst. In some embodiments, the total number of acidic monomers or moieties exceeds the total number of ionic monomers or moieties in the polymer or solid support. In other embodiments, the total number of acidic monomers or moieties is at least about 2, at least about 3, at least about 4, at least about 5, at least about 6, at least about 7, at least about 8, at least about 9 or at least about 10 times the total number of ionic monomers or moieties in the polymeric catalyst or solid-supported catalyst. In certain embodiments, the ratio of the total number of acidic monomers or moieties to the total number of ionic monomers or moieties is about 1:1, about 2:1, about 3:1, about 4:1, about 5:1, about 6:1, about 7:1, about 8:1, about 9:1 or about 10:1.

In some embodiments, the total number of ionic monomers or moieties exceeds the total number of acidic monomers or moieties in the catalyst. In other embodiments, the total number of ionic monomers or moieties is at least about 2, at least about 3, at least about 4, at least about 5, at least about 6, at least about 7, at least about 8, at least about 9 or at least about 10 times the total number of acidic monomers or moieties in the polymeric catalyst or solid-supported catalyst. In certain embodiments, the ratio of the total number of ionic monomers or moieties to the total number of acidic monomers or moieties is about 1:1, about 2:1, about 3:1, about 4:1, about 5:1, about 6:1, about 7:1, about 8:1, about 9:1 or about 10:1.

Arrangement of Monomers in Polymeric Catalysts

In some embodiments of the polymeric catalysts, the acidic monomers, the ionic monomers, the acidic-ionic monomers and the hydrophobic monomers, where present, can be arranged in alternating sequence or in a random order as blocks of monomers. In some embodiments, each block has not more than twenty, fifteen, ten, six, or three monomers.

In some embodiments of the polymeric catalysts, the monomers of the polymeric catalyst are randomly arranged in an alternating sequence. With reference to the portion of the polymeric catalyst depicted in FIG. 9, the monomers are randomly arranged in an alternating sequence.

In other embodiments of the polymeric catalysts, the monomers of the polymeric catalyst are randomly arranged as blocks of monomers. With reference to the portion of the polymeric catalyst depicted in FIG. 4, the monomers are arranged in blocks of monomers. In certain embodiments where the acidic monomers and the ionic monomers are arranged in blocks of monomers, each block has no more than 20, 19, 18, 17, 16, 15, 14, 13, 12, 11, 10, 9, 8, 7, 6, 5, 4, or 3 monomers.

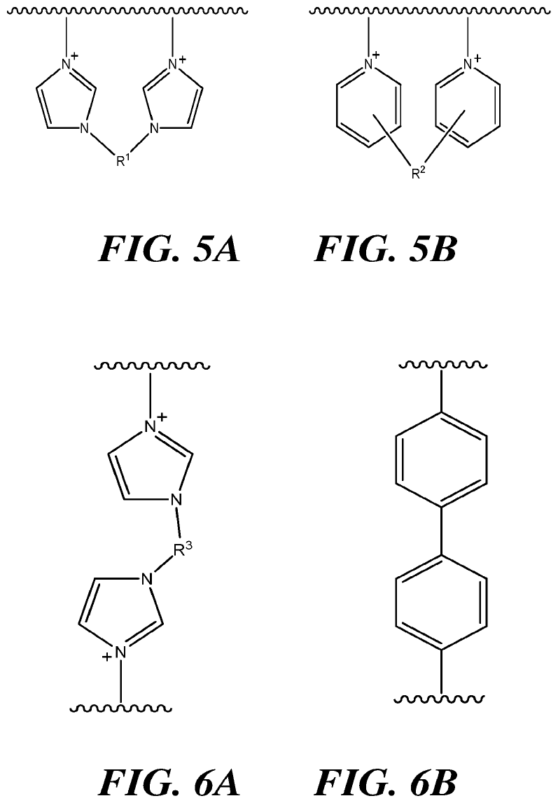

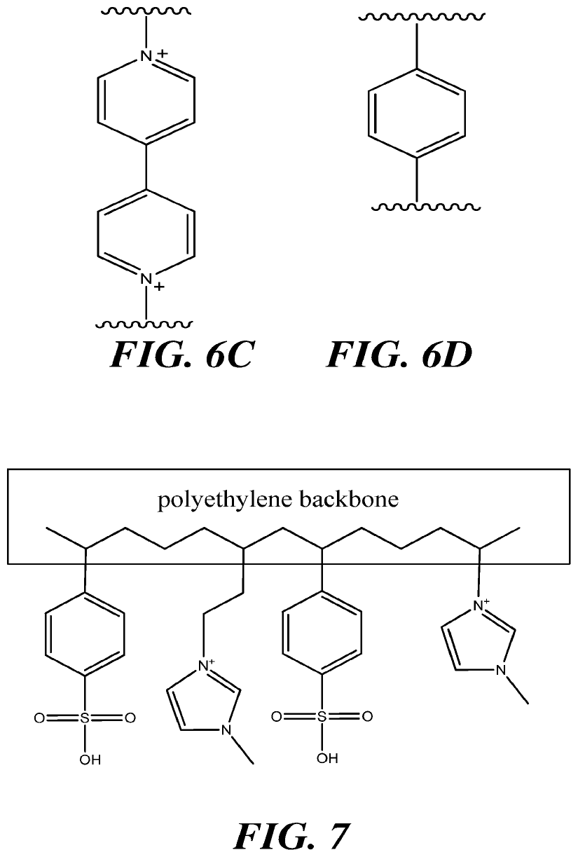

The polymeric catalysts described herein can also be cross-linked. Such cross-linked polymeric catalysts can be prepared by introducing cross-linking groups. In some embodiments, cross-linking can occur within a given polymeric chain, with reference to the portion of the polymeric catalysts depicted in FIGS. 5A and 5B. In other embodiments, cross-linking can occur between two or more polymeric chains, with reference to the portion of the polymeric catalysts in FIGS. 6A, 6B, 6C and 6D.

With reference to FIGS. 5A, 5B and 6A, it should be understood that R¹, R² and R³, respectively, are exemplary cross linking groups. Suitable cross-linking groups that can be used to form a cross-linked polymeric catalyst with the polymers described herein include, for example, substituted or unsubstituted divinyl alkanes, substituted or unsubstituted divinyl cycloalkanes, substituted or unsubstituted divinyl aryls, substituted or unsubstituted heteroaryls, dihaloalkanes, dihaloalkenes, and dihaloalkynes, where the substituents are those as defined herein. For example, cross-linking groups can include divinylbenzene, diallylbenzene, dichlorobenzene, divinylmethane, dichloromethane, divinylethane, dichloroethane, divinylpropane, dichloropropane, divinylbutane, dichlorobutane, ethylene glycol, and resorcinol. In one embodiment, the crosslinking group is divinyl benzene.

In some embodiments of the polymeric catalysts, the polymer is cross-linked. In certain embodiments, at least about 1%, at least about 2%, at least about 3%, at least about 4%, at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90% or at least about 99% of the polymer is cross-linked.

In some embodiments of the polymeric catalysts, the polymers described herein are not substantially cross-linked, such as less than about 0.9% cross-linked, less than about 0.5% cross-linked, less than about 0.1% cross-linked, less than about 0.01% cross-linked, or less than 0.001% cross-linked.

Polymeric Backbones

In some embodiments, the polymeric backbone is formed from one or more substituted or unsubstituted monomers. Polymerization processes using a wide variety of monomers are well known in the art (see, e.g., International Union of Pure and Applied Chemistry, et al., IUPAC Gold Book, Polymerization. (2000)). One such process involves monomer(s) with unsaturated substitution, such as vinyl, propenyl, butenyl, or other such substitutent(s). These types of monomers can undergo radical initiation and chain polymerization.

In some embodiments, the polymeric backbone is formed from one or more substituted or unsubstituted monomers selected from ethylene, propylene, hydroxyethylene, acetaldehyde, styrene, divinyl benzene, isocyanates, vinyl chloride, vinyl phenols, tetrafluoroethylene, butylene, terephthalic acid, caprolactam, acrylonitrile, butadiene, ammonias, diammonias, pyrrole, imidazole, pyrazole, oxazole, thiazole, pyridine, pyrimidine, pyrazine, pyridazine, thiazine, morpholine, piperidine, piperizines, pyrollizine, triphenylphosphonate, trimethylphosphonate, triethylphosphonate, tripropylphosphonate, tributylphosphonate, trichlorophosphonate, trifluorophosphonate, and diazole.

The polymeric backbone of the polymeric catalysts described herein can include, for example, polyalkylenes, polyalkenyl alcohols, polycarbonates, polyarylenes, polyaryletherketones, and polyamide-imides. In certain embodiments, the polymeric backbone can be selected from polyethylene, polypropylene, polyvinyl alcohol, polystyrene, polyurethane, polyvinyl chloride, polyphenol-aldehyde, polytetrafluoroethylene, polybutylene terephthalate, polycaprolactam, and poly(acrylonitrile butadiene styrene). In certain embodiments of the polymeric catalyst, the polymeric backbone is polyethyelene or polypropylene. In one embodiment of the polymeric catalyst, the polymeric backbone is polyethylene. In another embodiment of the polymeric catalyst, the polymeric backbone is polyvinyl alcohol. In yet another embodiment of the polymeric catalyst, the polymeric backbone is polystyrene.

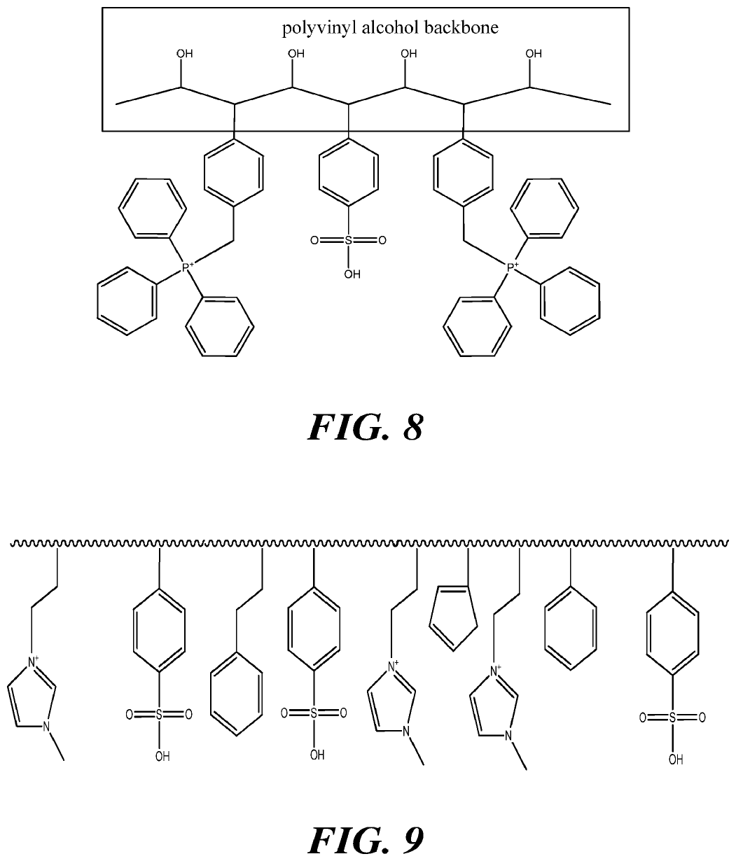

With reference to FIG. 7, in one embodiment, the polymeric backbone is polyethylene. With reference to FIG. 8, in another embodiment, the polymeric backbone is polyvinyl alcohol.

The polymeric backbone described herein can also include an ionic group integrated as part of the polymeric backbone. Such polymeric backbones can also be called “ionomeric backbones”. In certain embodiments, the polymeric backbone can be selected from: polyalkyleneammonium, polyalkylenediammonium, polyalkylenepyrrolium, polyalkyleneimidazolium, polyalkylenepyrazolium, polyalkyleneoxazolium, polyalkylenethiazolium, polyalkylenepyridinium, polyalkylenepyrimidinium, polyalkylenepyrazinium, polyalkylenepyridazinium, polyalkylenethiazinium, polyalkylenemorpholinium, polyalkylenepiperidinium, polyalkylenepiperizinium, polyalkylenepyrollizinium, polyalkylenetriphenylphosphonium, polyalkylenetrimethylphosphonium, polyalkylenetriethylphosphonium, polyalkylenetripropylphosphonium, polyalkylenetributylphosphonium, polyalkylenetrichlorophosphonium, polyalkylenetrifluorophosphonium, and polyalkylenediazolium, polyarylalkyleneammonium, polyarylalkylenediammonium, polyarylalkylenepyrrolium, polyarylalkyleneimidazolium, polyarylalkylenepyrazolium, polyarylalkyleneoxazolium, polyarylalkylenethiazolium, polyarylalkylenepyridinium, polyarylalkylenepyrimidinium, polyarylalkylenepyrazinium, polyarylalkylenepyridazinium, polyarylalkylenethiazinium, polyarylalkylenemorpholinium, polyarylalkylenepiperidinium, polyarylalkylenepiperizinium, polyarylalkylenepyrollizinium, polyarylalkylenetriphenylphosphonium, polyarylalkylenetrimethylphosphonium, polyarylalkylenetriethylphosphonium, polyarylalkylenetripropylphosphonium, polyarylalkylenetributylphosphonium, polyarylalkylenetrichlorophosphonium, polyarylalkylenetrifluorophosphonium, and polyarylalkylenediazolium.

Cationic polymeric backbones can be associated with one or more anions, including for example F⁻, Cl⁻, Br⁻, I⁻, NO₂⁻, NO₃⁻, SO₄²⁻, R⁷SO₄⁻, R⁷CO₂⁻, PO₄²⁻, R⁷PO₃⁻, and R⁷PO₂⁻, where R⁷ is selected from hydrogen, C₁₋₄alkyl, and C₁₋₄heteroalkyl. In one embodiment, each anion can be selected from Cl⁻, Br⁻, I⁻, HSO₄⁻, HCO₂⁻, CH₃CO₂⁻, and NO₃⁻. In other embodiments, each anion is acetate. In other embodiments, each anion is bisulfate. In other embodiments, each anion is chloride. In other embodiments, X is nitrate.

In other embodiments of the polymeric catalysts, the polymeric backbone is alkyleneimidazolium, which refers to an alkylene moiety, in which one or more of the methylene units of the alkylene moiety has been replaced with imidazolium. In one embodiment, the polymeric backbone is selected from polyethyleneimidazolium, polyprolyeneimidazolium, and polybutyleneimidazolium. It should further be understood that, in other embodiments of the polymeric backbone, when a nitrogen-containing cationic group or a phosphorous-containing cationic group follows the term “alkylene”, one or more of the methylene units of the alkylene moiety is substituted with that nitrogen-containing cationic group or phosphorous-containing cationic group.

In other embodiments, monomers having heteroatoms can be combined with one or more difunctionalized compounds, such as dihaloalkanes, di(alkylsulfonyloxy)alkanes, and di(arylsulfonyloxy)alkanes to form polymers. The monomers have at least two heteroatoms to link with the difunctionalized alkane to create the polymeric chain. These difunctionalized compounds can be further substituted as described herein. In some embodiments, the difunctionalized compound(s) can be selected from 1,2-dichloroethane, 1,2-dichloropropane, 1,3-dichloropropane, 1,2-dichlorobutane, 1,3-dichlorobutane, 1,4-dichlorobutane, 1,2-dichloropentane, 1,3-dichloropentane, 1,4-dichloropentane, 1,5-dichloropentane, 1,2-dibromoethane, 1,2-dibromopropane, 1,3-dibromopropane, 1,2-dibromobutane, 1,3-dibromobutane, 1,4-dibromobutane, 1,2-dibromopentane, 1,3-dibromopentane, 1,4-dibromopentane, 1,5-dibromopentane, 1,2-diiodoethane, 1,2-diiodopropane, 1,3-diiodopropane, 1,2-diiodobutane, 1,3-diiodobutane, 1,4-diiodobutane, 1,2-diiodopentane, 1,3-diiodopentane, 1,4-diiodopentane, 1,5-diiodopentane, 1,2-dimethanesulfoxyethane, 1,2-dimethanesulfoxypropane, 1,3-dimethanesulfoxypropane, 1,2-dimethanesulfoxybutane, 1,3-dimethanesulfoxybutane, 1,4-dimethanesulfoxybutane, 1,2-dimethanesulfoxypentane, 1,3-dimethanesulfoxypentane, 1,4-dimethanesulfoxypentane, 1,5-dimethanesulfoxypentane, 1,2-diethanesulfoxyethane, 1,2-diethanesulfoxypropane, 1,3-diethanesulfoxypropane, 1,2-diethanesulfoxybutane, 1,3-diethanesulfoxybutane, 1,4-diethanesulfoxybutane, 1,2-diethanesulfoxypentane, 1,3-diethanesulfoxypentane, 1,4-diethanesulfoxypentane, 1,5-diethanesulfoxypentane, 1,2-dibenzenesulfoxyethane, 1,2-dibenzenesulfoxypropane, 1,3-dibenzenesulfoxypropane, 1,2-dibenzenesulfoxybutane, 1,3-dibenzenesulfoxybutane, 1,4-dibenzenesulfoxybutane, 1,2-dibenzenesulfoxypentane, 1,3-dibenzenesulfoxypentane, 1,4-dibenzenesulfoxypentane, 1,5-dibenzenesulfoxypentane, 1,2-di-p-toluenesulfoxyethane, 1,2-di-p-toluenesulfoxypropane, 1,3-di-p-toluenesulfoxypropane, 1,2-di-p-toluenesulfoxybutane, 1,3-di-p-toluenesulfoxybutane, 1,4-di-p-toluenesulfoxybutane, 1,2-di-p-toluenesulfoxypentane, 1,3-di-p-toluene sulfoxypentane, 1,4-di-p-toluene sulfoxypentane, and 1,5-di-p-toluene sulfoxypentane.

Further, the number of atoms between side chains in the polymeric backbone can vary. In some embodiments, there are between zero and twenty atoms, zero and ten atoms, zero and six atoms, or zero and three atoms between side chains attached to the polymeric backbone.

In some embodiments, the polymer can be a homopolymer having at least two monomer units, and where all the units contained within the polymer are derived from the same monomer in the same manner. In other embodiments, the polymer can be a heteropolymer having at least two monomer units, and where at least one monomeric unit contained within the polymer that differs from the other monomeric units in the polymer. The different monomer units in the polymer can be in a random order, in an alternating sequence of any length of a given monomer, or in blocks of monomers.

Other exemplary polymers include, for example, polyalkylene backbones that are substituted with one or more groups selected from hydroxyl, carboxylic acid, unsubstituted and substituted phenyl, halides, unsubstituted and substituted amines, unsubstituted and substituted ammonias, unsubstituted and substituted pyrroles, unsubstituted and substituted imidazoles, unsubstituted and substituted pyrazoles, unsubstituted and substituted oxazoles, unsubstituted and substituted thiazoles, unsubstituted and substituted pyridines, unsubstituted and substituted pyrimidines, unsubstituted and substituted pyrazines, unsubstituted and substituted pyridazines, unsubstituted and substituted thiazines, unsubstituted and substituted morpholines, unsubstituted and substituted piperidines, unsubstituted and substituted piperizines, unsubstituted and substituted pyrollizines, unsubstituted and substituted triphenylphosphonates, unsubstituted and substituted trimethylphosphonates, unsubstituted and substituted triethylphosphonates, unsubstituted and substituted tripropylphosphonates, unsubstituted and substituted tributylphosphonates, unsubstituted and substituted trichlorophosphonates, unsubstituted and substituted trifluorophosphonates, and unsubstituted and substituted diazoles.

For the polymers as described herein, multiple naming conventions are well recognized in the art. For instance, a polyethylene backbone with a direct bond to an unsubstituted phenyl group (—CH₂—CH(phenyl)-CH₂—CH(phenyl)-) is also known as polystyrene. Should that phenyl group be substituted with an ethenyl group, the polymer can be named a polydivinylbenzene (—CH₂—CH(4-vinylphenyl)-CH₂—CH(4-vinylphenyl)-). Further examples of heteropolymers may include those that are functionalized after polymerization.

One suitable example would be polystyrene-co-divinylbenzene: (—CH₂—CH(phenyl)-CH₂—CH(4-ethylenephenyl)-CH₂—CH(phenyl)-CH₂—CH(4-ethylenephenyl)-). Here, the ethenyl functionality could be at the 2, 3, or 4 position on the phenyl ring.

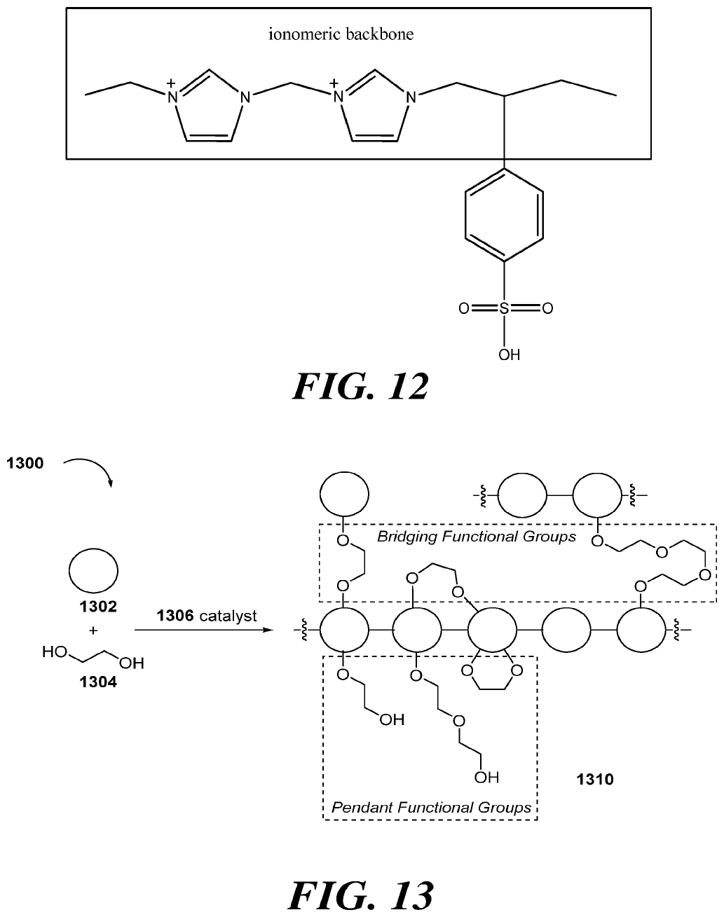

With reference to FIG. 12, in yet another embodiment, the polymeric backbone is a polyalkyleneimidazolium.

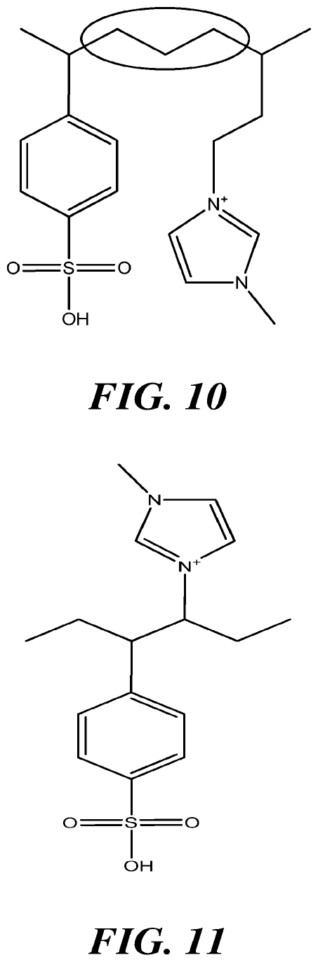

Further, the number of atoms between side chains in the polymeric backbone can vary. In some embodiments, there are between zero and twenty atoms, zero and ten atoms, or zero and six atoms, or zero and three atoms between side chains attached to the polymeric backbone. With reference to FIG. 10, in one embodiment, there are three carbon atoms between the side chain with the Bronsted-Lowry acid and the side chain with the cationic group. In another example, with reference to FIG. 11, there are zero atoms between the side chain with the acidic moiety and the side chain with the ionic moiety.

Solid Particles for Polymeric Catalysts

The polymeric catalysts described herein can form solid particles. One of skill in the art would recognize the various known techniques and methods to make solid particles from the polymers described herein. For example, a solid particle can be formed through the procedures of emulsion or dispersion polymerization, which are known to one of skill in the art. In other embodiments, the solid particles can be formed by grinding or breaking the polymer into particles, which are also techniques and methods that are known to one of skill in the art. Methods known in the art to prepare solid particles include coating the polymers described herein on the surface of a solid core. Suitable materials for the solid core can include an inert material (e.g., aluminum oxide, corn cob, crushed glass, chipped plastic, pumice, silicon carbide, or walnut shell) or a magnetic material. Polymeric coated core particles can be made by dispersion polymerization to grow a cross-linked polymer shell around the core material, or by spray coating or melting.

Other methods known in the art to prepare solid particles include coating the polymers described herein on the surface of a solid core. The solid core can be a non-catalytic support. Suitable materials for the solid core can include an inert material (e.g., aluminum oxide, corn cob, crushed glass, chipped plastic, pumice, silicon carbide, or walnut shell) or a magnetic material. In one embodiment of the polymeric catalyst, the solid core is made up of iron. Polymeric coated core particles can be made by techniques and methods that are known to one of skill in the art, for example, by dispersion polymerization to grow a cross-linked polymer shell around the core material, or by spray coating or melting.

The solid supported polymer catalyst particle can have a solid core where the polymer is coated on the surface of the solid core. In some embodiments, at least about 5%, at least about 10%, at least about 20%, at least about 30%, at least about 40%, or at least about 50% of the catalytic activity of the solid particle can be present on or near the exterior surface of the solid particle. In some embodiments, the solid core can have an inert material or a magnetic material. In one embodiment, the solid core is made up of iron.

The solid particles coated with the polymer described herein have one or more catalytic properties. In some embodiments, at least about 50%, at least about 60%, at least about 70%, at least about 80% or at least about 90% of the catalytic activity of the solid particle is present on or near the exterior surface of the solid particle.

In some embodiments, the solid particle is substantially free of pores, for example, having no more than about 50%, no more than about 40%, no more than about 30%, no more than about 20%, no more than about 15%, no more than about 10%, no more than about 5%, or no more than about 1% of pores. Porosity can be measured by methods well known in the art, such as determining the Brunauer-Emmett-Teller (BET) surface area using the absorption of nitrogen gas on the internal and external surfaces of a material (Brunauer, S. et al., J. Am. Chem. Soc. 1938, 60:309). Other methods include measuring solvent retention by exposing the material to a suitable solvent (such as water), then removing it thermally to measure the volume of interior pores. Other solvents suitable for porosity measurement of the polymeric catalysts include, for example, polar solvents such as DMF, DMSO, acetone, and alcohols.

In other embodiments, the solid particles include a microporous gel resin. In yet other embodiments, the solid particles include a macroporous gel resin.

Support of the Solid-Supported Catalysts

In certain embodiments of the solid-supported catalyst, the support may be selected from biochar, carbon, amorphous carbon, activated carbon, silica, silica gel, alumina, magnesia, titania, zirconia, clays (e.g., kaolinite), magnesium silicate, silicon carbide, zeolites (e.g., mordenite), ceramics, and any combinations thereof. In one embodiment, the support is carbon. The support for carbon support can be biochar, amorphous carbon, or activated carbon. In one embodiment, the support is activated carbon.

The carbon support can have a surface area from 0.01 to 50 m²/g of dry material. The carbon support can have a density from 0.5 to 2.5 kg/L. The support can be characterized using any suitable instrumental analysis methods or techniques known in the art, including for example scanning electron microscopy (SEM), powder X-ray diffraction (XRD), Raman spectroscopy, and Fourier Transform infrared spectroscopy (FTIR). The carbon support can be prepared from carbonaceous materials, including for example, shrimp shell, chitin, coconut shell, wood pulp, paper pulp, cotton, cellulose, hard wood, soft wood, wheat straw, sugarcane bagasse, cassava stem, corn stover, oil palm residue, bitumen, asphaltum, tar, coal, pitch, and any combinations thereof. One of skill in the art would recognize suitable methods to prepare the carbon supports used herein. See e.g., M. Inagaki, L. R. Radovic, Carbon, vol. 40, p. 2263 (2002), or A. G. Pandolfo and A. F. Hollenkamp, “Review: Carbon Properties and their role in supercapacitors,” Journal of Power Sources, vol. 157, pp. 11-27 (2006).

In other embodiments, the support is silica, silica gel, alumina, or silica-alumina. One of skill in the art would recognize suitable methods to prepare these silica- or alumina-based solid supports used herein. See e.g., Catalyst supports and supported catalysts, by A. B. Stiles, Butterworth Publishers, Stoneham Mass., 1987.

In yet other embodiments, the support is a combination of a carbon support, with one or more other supports selected from silica, silica gel, alumina, magnesia, titania, zirconia, clays (e.g., kaolinite), magnesium silicate, silicon carbide, zeolites (e.g., mordenite), and ceramics.

### Definitions

“Bronsted-Lowry acid” refers to a molecule, or substituent thereof, in neutral or ionic form that is capable of donating a proton (hydrogen cation, H⁺).

“Homopolymer” refers to a polymer having at least two monomer units, and where all the units contained within the polymer are derived from the same monomer. One suitable example is polyethylene, where ethylene monomers are linked to form a uniform repeating chain (—CH₂—CH₂—CH₂—). Another suitable example is polyvinyl chloride, having a structure (—CH₂—CHCl—CH₂—CHCl—) where the —CH₂—CHCl— repeating unit is derived from the H₂C═CHCl monomer.

“Heteropolymer” refers to a polymer having at least two monomer units, and where at least one monomeric unit differs from the other monomeric units in the polymer. Heteropolymer also refers to polymers having difunctionalized or trifunctionalized monomer units that can be incorporated in the polymer in different ways. The different monomer units in the polymer can be in a random order, in an alternating sequence of any length of a given monomer, or in blocks of monomers. One suitable example is polyethyleneimidazolium, where if in an alternating sequence, would be the polymer depicted in FIG. 12. Another suitable example is polystyrene-co-divinylbenzene, where if in an alternating sequence, could be (—CH₂—CH(phenyl)-CH₂—CH(4-ethylenephenyl)-CH₂—CH(phenyl)-CH₂—CH(4-ethylenephenyl)-). Here, the ethenyl functionality could be at the 2, 3, or 4 position on the phenyl ring.

As used herein, [symbol] denotes the attachment point of a moiety to the parent structure.

When a range of values is listed, it is intended to encompass each value and sub-range within the range. For example, “C₁₋₆ alkyl” (which may also be referred to as 1-6C alkyl, C1-C6 alkyl, or C1-6 alkyl) is intended to encompass, C₁, C₂, C₃, C₄, C₅, C₆, C₁₋₆, C₁₋₅, C₁₋₄, C₁₋₃, C₁₋₂, C₂₋₆, C₂-s, C₂₋₄, C₂₋₃, C₃₋₆, C₃₋₅, C₃₋₄, C₄₋₆, C₄₋₅, and C₅₋₆ alkyl.

“Alkyl” includes saturated straight-chained or branched monovalent hydrocarbon radicals, which contain only C and H when unsubstituted. In some embodiments, alkyl as used herein may have 1 to 10 carbon atoms (e.g., C₁₋₁₀ alkyl), 1 to 6 carbon atoms (e.g., C1-6 alkyl), or 1 to 3 carbon atoms (e.g., C1-3 alkyl). Representative straight-chained alkyls include, for example, methyl, ethyl, n-propyl, n-butyl, n-pentyl, and n-hexyl. Representative branched alkyls include, for example, isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, and 2,3-dimethylbutyl. When an alkyl residue having a specific number of carbons is named, all geometric isomers having that number of carbons are intended to be encompassed and described; thus, for example, “butyl” is meant to include n-butyl, sec-butyl, iso-butyl, and tert-butyl; “propyl” includes n-propyl, and iso-propyl.

“Alkoxy” refers to the group —O-alkyl, which is attached to the parent structure through an oxygen atom. Examples of alkoxy may include methoxy, ethoxy, propoxy, and isopropoxy. In some embodiments, alkoxy as used herein has 1 to 6 carbon atoms (e.g., O—(C₁₋₆ alkyl)), or 1 to 4 carbon atoms (e.g., O—(C₁₋₄ alkyl)).

“Alkenyl” refers to straight-chained or branched monovalent hydrocarbon radicals, which contain only C and H when unsubstituted and at least one double bond. In some embodiments, alkenyl has 2 to 10 carbon atoms (e.g., C₂₋₁₀ alkenyl), or 2 to 5 carbon atoms (e.g., C2-s alkenyl). When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl may include —CH═CH₂, —CH₂—CH═CH₂ and —CH₂—CH═CH—CH═CH₂. The one or more carbon-carbon double bonds can be internal (such as in 2-butenyl) or terminal (such as in 1-butenyl). Examples of C2-4 alkenyl groups include ethenyl (C2), 1-propenyl (C3), 2-propenyl (C3), 1-butenyl (C4), 2-butenyl (C4), and butadienyl (C4). Examples of C2-6 alkenyl groups include the aforementioned C2-4 alkenyl groups as well as pentenyl (C5), pentadienyl (C5), and hexenyl (C6). Additional examples of alkenyl include heptenyl (C7), octenyl (C8), and octatrienyl (C8).

“Alkynyl” refers to straight-chained or branched monovalent hydrocarbon radicals, which contain only C and H when unsubstituted and at least one triple bond. In some embodiments, alkynyl has 2 to 10 carbon atoms (e.g., C₂₋₁₀ alkynyl), or 2 to 5 carbon atoms (e.g., C2-s alkynyl). When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl may include —C≡CH or —C≡C—CH₃.

In some embodiments, alkyl, alkoxy, alkenyl, and alkynyl at each occurrence may independently be unsubstituted or substituted by one or more of substituents. In certain embodiments, substituted alkyl, substituted alkoxy, substituted alkenyl, and substituted alkynyl at each occurrence may independently have 1 to 5 substituents, 1 to 3 substituents, 1 to 2 substituents, or 1 substituent. Examples of alkyl, alkoxy, alkenyl, and alkynyl substituents may include alkoxy, cycloalkyl, aryl, aryloxy, amino, amido, carbamate, carbonyl, oxo (═O), heteroalkyl (e.g., ether), heteroaryl, heterocycloalkyl, cyano, halo, haloalkoxy, haloalkyl, and thio. In certain embodiments, the one or more substituents of substituted alkyl, alkoxy, alkenyl, and alkynyl is independently selected from cycloalkyl, aryl, heteroalkyl (e.g., ether), heteroaryl, heterocycloalkyl, cyano, halo, haloalkoxy, haloalkyl, oxo, —ORₐ, —N(Rₐ)₂, —C(O)N(Rₐ)₂, —N(Rₐ)C(O)Rₐ, —C(O)Rₐ, —N(Rₐ)S(O)ₜRₐ (where t is 1 or 2), —SRₐ, and —S(O)ₜN(Rₐ)₂ (where t is 1 or 2). In certain embodiments, each Rₐ is independently hydrogen, alkyl, alkenyl, alkynyl, haloalkyl, heteroalkyl, cycloalkyl, aryl, heterocycloalkyl, heteroaryl (e.g., bonded through a ring carbon), —C(O)R′ and —S(O)ₜR′ (where t is 1 or 2), where each R′ is independently hydrogen, alkyl, alkenyl, alkynyl, haloalkyl, heteroalkyl, cycloalkyl, aryl, heterocycloalkyl, or heteroaryl. In one embodiment, Rₐ is independently hydrogen, alkyl, haloalkyl, cycloalkyl, aryl, aralkyl (e.g., alkyl substituted with aryl, bonded to parent structure through the alkyl group), heterocycloalkyl, or heteroaryl.

“Heteroalkyl”, “heteroalkenyl” and “heteroalkynyl” includes alkyl, alkenyl and alkynyl groups, respectively, wherein one or more skeletal chain atoms are selected from an atom other than carbon, e.g., oxygen, nitrogen, sulfur, phosphorus, or any combinations thereof. For example, heteroalkyl may be an ether where at least one of the carbon atoms in the alkyl group is replaced with an oxygen atom. A numerical range can be given, e.g., CIA heteroalkyl which refers to the chain length in total, which in this example is 4 atoms long. For example, a —CH₂OCH₂CH₃ group is referred to as a “Ca” heteroalkyl, which includes the heteroatom center in the atom chain length description. Connection to the rest of the parent structure can be through, in one embodiment, a heteroatom, or, in another embodiment, a carbon atom in the heteroalkyl chain. Heteroalkyl groups may include, for example, ethers such as methoxyethanyl (—CH₂CH₂OCH₃), ethoxymethanyl (—CH₂OCH₂CH₃), (methoxymethoxy)ethanyl (—CH₂CH₂OCH₂OCH₃), (methoxymethoxy)methanyl (—CH₂OCH₂OCH₃) and (methoxyethoxy)methanyl (—CH₂OCH₂CH₂OCH₃); amines such as —CH₂CH₂NHCH₃, —CH₂CH₂N(CH₃)₂, —CH₂NHCH₂CH₃, and —CH₂N(CH₂CH₃)(CH₃). In some embodiments, heteroalkyl, heteroalkenyl, or heteroalkynyl may be unsubstituted or substituted by one or more of substituents. In certain embodiments, a substituted heteroalkyl, heteroalkenyl, or heteroalkynyl may have 1 to 5 substituents, 1 to 3 substituents, 1 to 2 substituents, or 1 substituent. Examples for heteroalkyl, heteroalkenyl, or heteroalkynyl substituents may include the substituents described above for alkyl.

“Carbocyclyl” may include cycloalkyl, cycloalkenyl or cycloalkynyl. “Cycloalkyl” refers to a monocyclic or polycyclic alkyl group. “Cycloalkenyl” refers to a monocyclic or polycyclic alkenyl group (e.g., containing at least one double bond). “Cycloalkynyl” refers to a monocyclic or polycyclic alkynyl group (e.g., containing at least one triple bond). The cycloalkyl, cycloalkenyl, or cycloalkynyl can consist of one ring, such as cyclohexyl, or multiple rings, such as adamantyl. A cycloalkyl, cycloalkenyl, or cycloalkynyl with more than one ring can be fused, spiro or bridged, or combinations thereof. In some embodiments, cycloalkyl, cycloalkenyl, and cycloalkynyl has 3 to 10 ring atoms (i.e., C₃-C₁₀cycloalkyl, C₃-C₁₀ cycloalkenyl, and C₃-C₁₀ cycloalkynyl), 3 to 8 ring atoms (e.g., C₃-C₈ cycloalkyl, C₃-C₈ cycloalkenyl, and C₃-C₈ cycloalkynyl), or 3 to 5 ring atoms (i.e., C₃-C₅ cycloalkyl, C₃-C₅ cycloalkenyl, and C₃-C₅ cycloalkynyl). In certain embodiments, cycloalkyl, cycloalkenyl, or cycloalkynyl includes bridged and spiro-fused cyclic structures containing no heteroatoms. In other embodiments, cycloalkyl, cycloalkenyl, or cycloalkynyl includes monocyclic or fused-ring polycyclic (i.e., rings which share adjacent pairs of ring atoms) groups. C₃₋₆ carbocyclyl groups may include, for example, cyclopropyl (C₃), cyclobutyl (C₄), cyclopentyl (C₅), cyclopentenyl (C₅), cyclohexyl (C₆), cyclohexenyl (C₆), and cyclohexadienyl (C₆). C₃₋₈ carbocyclyl groups may include, for example, the aforementioned C₃₋₆ carbocyclyl groups as well as cycloheptyl (C₇), cycloheptadienyl (C₇), cycloheptatrienyl (C₇), cyclooctyl (C₈), bicyclo[2.2.1]heptanyl, and bicyclo[2.2.2]octanyl. C₃₋₁₀ carbocyclyl groups may include, for example, the aforementioned C₃₋₈ carbocyclyl groups as well as octahydro-1H-indenyl, decahydronaphthalenyl, and spiro[4.5]decanyl.

“Heterocyclyl” refers to carbocyclyl as described above, with one or more ring heteroatoms independently selected from nitrogen, oxygen, phosphorous, and sulfur. Heterocyclyl may include, for example, heterocycloalkyl, heterocycloalkenyl, and heterocycloalknyl. In some embodiments, heterocyclyl is a 3- to 18-membered non-aromatic monocyclic or polycyclic moiety that has at least one heteroatom selected from nitrogen, oxygen, phosphorous and sulfur. In certain embodiments, the heterocyclyl can be a monocyclic or polycyclic (e.g., bicyclic, tricyclic or tetracyclic), wherein polycyclic ring systems can be a fused, bridged or spiro ring system. Heterocyclyl polycyclic ring systems can include one or more heteroatoms in one or both rings.

An N-containing heterocyclyl moiety refers to an non-aromatic group in which at least one of the skeletal atoms of the ring is a nitrogen atom. The heteroatom(s) in the heterocyclyl group is optionally oxidized. One or more nitrogen atoms, if present, are optionally quaternized. In certain embodiments, heterocyclyl may also include ring systems substituted with one or more oxide (—O—) substituents, such as piperidinyl N-oxides. The heterocyclyl is attached to the parent molecular structure through any atom of the ring(s).

In some embodiments, heterocyclyl also includes ring systems with one or more fused carbocyclyl, aryl or heteroaryl groups, wherein the point of attachment is either on the carbocyclyl or heterocyclyl ring. In some embodiments, heterocyclyl is a 5-10 membered non-aromatic ring system having ring carbon atoms and 1-4 ring heteroatoms, wherein each heteroatom is independently selected from nitrogen, oxygen and sulfur (e.g., 5-10 membered heterocyclyl). In some embodiments, a heterocyclyl group is a 5-8 membered non-aromatic ring system having ring carbon atoms and 1-4 ring heteroatoms, wherein each heteroatom is independently selected from nitrogen, oxygen and sulfur (e.g., 5-8 membered heterocyclyl). In some embodiments, a heterocyclyl group is a 5-6 membered non-aromatic ring system having ring carbon atoms and 1-4 ring heteroatoms, wherein each heteroatom is independently selected from nitrogen, oxygen and sulfur (e.g., 5-6 membered heterocyclyl). In some embodiments, the 5-6 membered heterocyclyl has 1-3 ring heteroatoms selected from nitrogen, oxygen and sulfur. In some embodiments, the 5-6 membered heterocyclyl has 1-2 ring heteroatoms selected from nitrogen, oxygen and sulfur. In some embodiments, the 5-6 membered heterocyclyl has 1 ring heteroatom selected from nitrogen, oxygen and sulfur.

“Aryl” refers to an aromatic group having a single ring (e.g., phenyl), multiple rings (e.g., biphenyl), or multiple fused rings (e.g., naphthyl, fluorenyl, and anthryl). In some embodiments, aryl as used herein has 6 to 10 ring atoms (e.g., C₆-C₁₀ aromatic or C₆-C₁₀ aryl) which has at least one ring having a conjugated pi electron system. For example, bivalent radicals formed from substituted benzene derivatives and having the free valences at ring atoms are named as substituted phenylene radicals. In certain embodiments, aryl may have more than one ring where at least one ring is non-aromatic can be connected to the parent structure at either an aromatic ring position or at a non-aromatic ring position. In certain embodiments, aryl includes monocyclic or fused-ring polycyclic (i.e., rings which share adjacent pairs of ring atoms) groups.

“Heteroaryl” refers to an aromatic group having a single ring, multiple rings, or multiple fused rings, with one or more ring heteroatoms independently selected from nitrogen, oxygen, phosphorous, and sulfur. In some embodiments, heteroaryl is an aromatic, monocyclic or bicyclic ring containing one or more heteroatoms independently selected from nitrogen, oxygen and sulfur with the remaining ring atoms being carbon. In certain embodiments, heteroaryl is a 5- to 18-membered monocyclic or polycyclic (e.g., bicyclic or tricyclic) aromatic ring system (e.g., having 6, 10 or 14 pi electrons shared in a cyclic array) having ring carbon atoms and 1 to 6 ring heteroatoms provided in the aromatic ring system, wherein each heteroatom is independently selected from nitrogen, oxygen, phosphorous and sulfur (e.g., 5-18 membered heteroaryl). In certain embodiments, heteroaryl may have a single ring (e.g., pyridyl, pyridinyl, imidazolyl) or multiple condensed rings (e.g., indolizinyl, benzothienyl) which condensed rings may or may not be aromatic. In other embodiments, heteroaryl may have more than one ring where at least one ring is non-aromatic can be connected to the parent structure at either an aromatic ring position or at a non-aromatic ring position. In one embodiment, heteroaryl may have more than one ring where at least one ring is non-aromatic is connected to the parent structure at an aromatic ring position. Heteroaryl polycyclic ring systems can include one or more heteroatoms in one or both rings.

For example, in one embodiment, an N-containing “heteroaryl” refers to an aromatic group in which at least one of the skeletal atoms of the ring is a nitrogen atom. One or more heteroatom(s) in the heteroaryl group can be optionally oxidized. One or more nitrogen atoms, if present, are optionally quaternized. In other embodiments, heteroaryl may include ring systems substituted with one or more oxide (—O—) substituents, such as pyridinyl N-oxides. The heteroaryl may be attached to the parent molecular structure through any atom of the ring(s).

In other embodiments, heteroaryl may include ring systems with one or more fused aryl groups, wherein the point of attachment is either on the aryl or on the heteroaryl ring. In yet other embodiments, heteroaryl may include ring systems with one or more carbocyclyl or heterocycyl groups wherein the point of attachment is on the heteroaryl ring. For polycyclic heteroaryl groups wherein one ring does not contain a heteroatom (e.g., indolyl, quinolinyl, and carbazolyl) the point of attachment can be on either ring, i.e., either the ring bearing a heteroatom (e.g., 2-indolyl) or the ring that does not contain a heteroatom (e.g., 5-indolyl). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, phosphorous, and sulfur (e.g., 5-10 membered heteroaryl). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, phosphorous, and sulfur (e.g., 5-8 membered heteroaryl). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, phosphorous, and sulfur (e.g., 5-6 membered heteroaryl). In some embodiments, the 5-6 membered heteroaryl has 1-3 ring heteroatoms selected from nitrogen, oxygen, phosphorous, and sulfur. In some embodiments, the 5-6 membered heteroaryl has 1-2 ring heteroatoms selected from nitrogen, oxygen, phosphorous, and sulfur. In some embodiments, the 5-6 membered heteroaryl has 1 ring heteroatom selected from nitrogen, oxygen, phosphorous, and sulfur.

In some embodiments, carbocyclyl (including, for example, cycloalkyl, cycloalkenyl or cycloalkynyl), aryl, heteroaryl, and heterocyclyl at each occurrence may independently be unsubstituted or substituted by one or more of substituents. In certain embodiments, a substituted carbocyclyl (including, for example, substituted cycloalkyl, substituted cycloalkenyl or substituted cycloalkynyl), substituted aryl, substituted heteroaryl, substituted heterocyclyl at each occurrence may be independently may independently have 1 to 5 substituents, 1 to 3 substituents, 1 to 2 substituents, or 1 substituent.

Examples of carbocyclyl (including, for example, cycloalkyl, cycloalkenyl or cycloalkynyl), aryl, heteroaryl, heterocyclyl substituents may include alkyl alkenyl, alkoxy, cycloalkyl, aryl, heteroalkyl (e.g., ether), heteroaryl, heterocycloalkyl, cyano, halo, haloalkoxy, haloalkyl, oxo (═O), —ORₐ, —N(Rₐ)₂, —C(O)N(Rₐ)₂, —N(Rₐ)C(O)Rₐ, —C(O)Rₐ, —N(Rₐ)S(O)ₜRₐ (where t is 1 or 2), —SRₐ, and —S(O)ₜN(Rₐ)₂ (where t is 1 or 2), wherein Rₐ is as described herein.

It should be understood that, as used herein, any moiety referred to as a “linker” refers to the moiety has having bivalency. Thus, for example, “alkyl linker” refers to the same residues as alkyl, but having bivalency. Examples of alkyl linkers include —CH₂-, —CH₂CH₂-, —CH₂CH₂CH₂-, and —CH₂CH₂CH₂CH₂-. “Alkenyl linker” refers to the same residues as alkenyl, but having bivalency. Examples of alkenyl linkers include —CH═CH—, —CH₂—CH═CH— and —CH₂—CH═CH—CH₂—. “Alkynyl linker” refers to the same residues as alkynyl, but having bivalency. Examples alkynyl linkers include —C≡C— or —C≡C—CH₂—. Similarly, “carbocyclyl linker”, “aryl linker”, “heteroaryl linker”, and “heterocyclyl linker” refer to the same residues as carbocyclyl, aryl, heteroaryl, and heterocyclyl, respectively, but having bivalency.

“Amino” or “amine” refers to —N(Rₐ)(R_{b}), where each Rₐ and R_{b} is independently selected from hydrogen, alkyl, alkenyl, alkynyl, haloalkyl, heteroalkyl (e.g., bonded through a chain carbon), cycloalkyl, aryl, heterocycloalkyl (e.g., bonded through a ring carbon), heteroaryl (e.g., bonded through a ring carbon), —C(O)R′ and —S(O)ₜR′ (where t is 1 or 2), where each R′ is independently hydrogen, alkyl, alkenyl, alkynyl, haloalkyl, heteroalkyl, cycloalkyl, aryl, heterocycloalkyl, or heteroaryl. It should be understood that, in one embodiment, amino includes amido (e.g., —NRₐC(O)R_{b}). It should be further understood that in certain embodiments, the alkyl, alkenyl, alkynyl, haloalkyl, heteroalkyl, cycloalkyl, aryl, heterocycloalkyl, or heteroaryl moiety of Rₐ and R_{b} may be further substituted as described herein. Rₐ and R_{b} may be the same or different. For example, in one embodiment, amino is —NH₂ (where Rₐ and R_{b} are each hydrogen). In other embodiments where Rₐ and R_{b} are other than hydrogen, Rₐ and R_{b} can be combined with the nitrogen atom to which they are attached to form a 3-, 4-, 5-, 6-, or 7-membered ring. Such examples may include 1-pyrrolidinyl and 4-morpholinyl.

“Ammonium” refers to —N(Rₐ)(R_{b})(R_{c})⁺, where each Rₐ, R_{b} and R_{c} is independently selected from hydrogen, alkyl, alkenyl, alkynyl, haloalkyl, heteroalkyl (e.g., bonded through a chain carbon), cycloalkyl, aryl, heterocycloalkyl (e.g., bonded through a ring carbon), heteroaryl (e.g., bonded through a ring carbon), —C(O)R′ and —S(O)ₜR′ (where t is 1 or 2), where each R′ is independently hydrogen, alkyl, alkenyl, alkynyl, haloalkyl, heteroalkyl, cycloalkyl, aryl, heterocycloalkyl, or heteroaryl; or any two of Rₐ, R_{b} and R_{c} may be taken together with the atom to which they are attached to form a cycloalkyl, heterocycloalkyl; or any three of Rₐ, R_{b} and R_{c} may be taken together with the atom to which they are attached to form aryl or heteroaryl. It should be further understood that in certain embodiments, the alkyl, alkenyl, alkynyl, haloalkyl, heteroalkyl, cycloalkyl, aryl, heterocycloalkyl, or heteroaryl moiety of any one or more of Rₐ, R_{b} and R_{c} may be further substituted as described herein. Rₐ, R_{b} and R_{c} may be the same or different.

In certain embodiments, “amino” also refers to N-oxides of the groups —N⁺(H)(Rₐ)O⁻, and —N⁺(Rₐ)(R_{b})O—, where Rₐ and R_{b} are as described herein, where the N-oxide is bonded to the parent structure through the N atom. N-oxides can be prepared by treatment of the corresponding amino group with, for example, hydrogen peroxide or m-chloroperoxybenzoic acid. The person skilled in the art is familiar with reaction conditions for carrying out the N-oxidation.

“Amide” or “amido” refers to a chemical moiety with formula —C(O) N(Rₐ)(R_{b}) or —NRₐC(O)R_{b}, where Rₐ and R_{b} at each occurrence are as described herein. In some embodiments, amido is a C1-4 amido, which includes the amide carbonyl in the total number of carbons in the group. When a —C(O) N(Rₐ)(R_{b}) has Rₐ and R_{b} other than hydrogen, they can be combined with the nitrogen atom to form a 3-, 4-, 5-, 6-, or 7-membered ring.

“Carbonyl” refers to —C(O)Rₐ, where Rₐ is hydrogen, alkyl, alkenyl, alkynyl, haloalkyl, heteroalkyl, cycloalkyl, aryl, heterocycloalkyl, heteroaryl, —N(R′)₂, —S(O)ₜR′, where each R′ is independently hydrogen, alkyl, alkenyl, alkynyl, haloalkyl, heteroalkyl, cycloalkyl, aryl, heterocycloalkyl, or heteroaryl, and t is 1 or 2. In certain embodiments where each R′ are other than hydrogen, the two R′ moieties can be combined with the nitrogen atom to which they are attached to form a 3-, 4-, 5-, 6-, or 7-membered ring. It should be understood that, in one embodiment, carbonyl includes amido (e.g., —C(O) N(Rₐ)(R_{b})).

“Carbamate” refers to any of the following groups: —O—C(═O)—N(Rₐ)(R_{b}) and —N(Rₐ)—C(═O)—OR_{b}, wherein Rₐ and R_{b} at each occurrence are as described herein.

“Cyano” refers to a —CN group.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy moieties as described above, wherein one or more hydrogen atoms are replaced by halo. For example, where a residue is substituted with more than one halo groups, it may be referred to by using a prefix corresponding to the number of halo groups attached. For example, dihaloaryl, dihaloalkyl, and trihaloaryl refer to aryl and alkyl substituted with two (“di”) or three (“tri”) halo groups, which may be, but are not necessarily, the same halogen; thus, for example, 3,5-difluorophenyl, 3-chloro-5-fluorophenyl, 4-chloro-3-fluorophenyl, and 3,5-difluoro-4-chlorophenyl is within the scope of dihaloaryl. Other examples of a haloalkyl group include difluoromethyl (˜CHF₂), trifluoromethyl (˜CF₃), 2,2,2-trifluoroethyl, and 1-fluoromethyl-2-fluoroethyl. Each of the alkyl, alkenyl, alkynyl and alkoxy groups of haloalkyl, haloalkenyl, haloalkynyl and haloalkoxy, respectively, can be optionally substituted as defined herein. “Perhaloalkyl” refers to an alkyl or alkylene group in which all of the hydrogen atoms have been replaced with a halogen (e.g., fluoro, chloro, bromo, or iodo). In some embodiments, all of the hydrogen atoms are each replaced with fluoro. In some embodiments, all of the hydrogen atoms are each replaced with chloro. Examples of perhaloalkyl groups include —CF₃, —CF₂CF₃, —CF₂CF₂CF₃, —CCl₃, —CFCl₂, and —CF₂Cl.

“Thio” refers to —SRₐ, wherein Rₐ is as described herein. “Thiol” refers to the group —RₐSH, wherein Rₐ is as described herein.

“Sulfinyl” refers to —S(O)Rₐ. In some embodiments, sulfinyl is —S(O)N(Rₐ)(R_{b}). “Sulfonyl” refers to the —S(O₂)Rₐ. In some embodiments, sulfonyl is —S(O₂) N(Rₐ)(R_{b}) or —S(O₂)OH. For each of these moieties, it should be understood that Rₐ and R_{b} are as described herein.

“Moiety” refers to a specific segment or functional group of a molecule. Chemical moieties are often recognized chemical entities embedded in or appended to a molecule.

As used herein, the term “unsubstituted” means that for carbon atoms, only hydrogen atoms are present besides those valencies linking the atom to the parent molecular group. One example is propyl (—CH₂—CH₂—CH₃). For nitrogen atoms, valencies not linking the atom to the parent molecular group are either hydrogen or an electron pair. For sulfur atoms, valencies not linking the atom to the parent molecular group are either hydrogen, oxygen or electron pair(s).

As used herein, the term “substituted” or “substitution” means that at least one hydrogen present on a group (e.g., a carbon or nitrogen atom) is replaced with a permissible substituent, e.g., a substituent which upon substitution for the hydrogen results in a stable compound, e.g., a compound which does not spontaneously undergo transformation such as by rearrangement, cyclization, elimination, or other reaction. Unless otherwise indicated, a “substituted” group can have a substituent at one or more substitutable positions of the group, and when more than one position in any given structure is substituted, the substituent is either the same or different at each position. Substituents include one or more group(s) individually and independently selected from alkyl alkenyl, alkoxy, cycloalkyl, aryl, heteroalkyl (e.g., ether), heteroaryl, heterocycloalkyl, cyano, halo, haloalkoxy, haloalkyl, oxo (═O), —ORₐ, —N(Rₐ)₂, —C(O)N(Rₐ)₂, —N(Rₐ)C(O)Rₐ, —C(O)Rₐ, —N(Rₐ)S(O)ₜRₐ (where t is 1 or 2), —SRₐ, and —S(O)ₜN(Rₐ)₂ (where t is 1 or 2), wherein Rₐ is as described herein.

Where substituent groups are specified by their conventional chemical formulae, written from left to right, they equally encompass the chemically identical substituents that would result from writing the structure from right to left, e.g., —CH₂O— is equivalent to —OCH₂—.

Unless defined otherwise, all technical and scientific terms used herein have the same meaning as is commonly understood by one of skill in the art to which this specification pertains.

As used in the specification and claims, the singular form “a”, “an” and “the” includes plural references unless the context clearly dictates otherwise.

Reference to “about” a value or parameter herein includes (and describes) embodiments that are directed to that value or parameter per se. For example, description referring to “about x” includes description of “x” per se. In other instances, the term “about” when used in association with other measurements, or used to modify a value, a unit, a constant, or a range of values, refers to variations of between ±0.1% and ±15% of the stated number. For example, in one variation, “about 1” refers to a range between 0.85 and 1.15.

Reference to “between” two values or parameters herein includes (and describes) embodiments that include those two values or parameters per se. For example, description referring to “between x and y” includes description of “x” and “y” per se.

Representative Examples of Catalysts for Use in Producing Oligosaccharide Compositions

It should be understood that the polymeric catalysts and the solid-supported catalysts can include any of the Bronsted-Lowry acids, cationic groups, counterions, linkers, hydrophobic groups, cross-linking groups, and polymeric backbones or solid supports (as the case may be) described herein, as if each and every combination were listed separately. For example, in one embodiment, the catalyst can include benzenesulfonic acid (i.e., a sulfonic acid with a phenyl linker) connected to a polystyrene backbone or attached to the solid support, and an imidazolium chloride connected directly to the polystyrene backbone or attached directly to the solid support. In another embodiment, the polymeric catalyst can include boronyl-benzyl-pyridinium chloride (i.e., a boronic acid and pyridinium chloride in the same monomer unit with a phenyl linker) connected to a polystyrene backbone or attached to the solid support. In yet another embodiment, the catalyst can include benzenesulfonic acid and imidazolium sulfate each individually connected to a polyvinyl alcohol backbone or individually attached to the solid support.

In some embodiments, the polymeric catalyst is selected from:
poly [styrene-co-4-vinylbenzenesulfonic acid-co-3-methyl-1-(4-vinylbenzyl)-3H-imidazol-1-ium chloride-co-divinylbenzene];
poly [styrene-co-4-vinylbenzenesulfonic acid-co-3-methyl-1-(4-vinylbenzyl)-3H-imidazol-1-ium bisulfate-co-divinylbenzene];
poly [styrene-co-4-vinylbenzenesulfonic acid-co-3-methyl-1-(4-vinylbenzyl)-3H-imidazol-1-ium acetate-co-divinylbenzene];
poly [styrene-co-4-vinylbenzenesulfonic acid-co-3-methyl-1-(4-vinylbenzyl)-3H-imidazol-1-ium nitrate-co-divinylbenzene];
poly [styrene-co-4-vinylbenzenesulfonic acid-co-3-ethyl-1-(4-vinylbenzyl)-3H-imidazol-1-ium chloride-co-divinylbenzene];
poly [styrene-co-4-vinylbenzenesulfonic acid-co-3-ethyl-1-(4-vinylbenzyl)-3H-imidazol-1-ium bisulfate-co-divinylbenzene];
poly [styrene-co-4-vinylbenzenesulfonic acid-co-3-ethyl-1-(4-vinylbenzyl)-3H-imidazol-1-ium acetate-co-divinylbenzene];
poly [styrene-co-4-vinylbenzenesulfonic acid-co-3-ethyl-1-(4-vinylbenzyl)-3H-imidazol-1-ium nitrate-co-divinylbenzene];
poly [styrene-co-4-vinylbenzenesulfonic acid-co-1-(4-vinylbenzyl)-3H-imidazol-1-ium chloride-co-divinylbenzene];
poly [styrene-co-4-vinylbenzenesulfonic acid-co-1-(4-vinylbenzyl)-3H-imidazol-1-ium iodide-co-divinylbenzene];
poly [styrene-co-4-vinylbenzenesulfonic acid-co-1-(4-vinylbenzyl)-3H-imidazol-1-ium bromide-co-divinylbenzene];
poly [styrene-co-4-vinylbenzenesulfonic acid-co-1-(4-vinylbenzyl)-3H-imidazol-1-ium bisulfate-co-divinylbenzene];
poly [styrene-co-4-vinylbenzenesulfonic acid-co-1-(4-vinylbenzyl)-3H-imidazol-1-ium acetate-co-divinylbenzene];
poly [styrene-co-4-vinylbenzenesulfonic acid-co-3-methyl-1-(4-vinylbenzyl)-3H-benzoimidazol-1-ium chloride-co-divinylbenzene];
poly [styrene-co-4-vinylbenzenesulfonic acid-co-3-methyl-1-(4-vinylbenzyl)-3H-benzoimidazol-1-ium bisulfate-co-divinylbenzene];
poly [styrene-co-4-vinylbenzenesulfonic acid-co-3-methyl-1-(4-vinylbenzyl)-3H-benzoimidazol-1-ium acetate-co-divinylbenzene];
poly [styrene-co-4-vinylbenzenesulfonic acid-co-3-methyl-1-(4-vinylbenzyl)-3H-benzoimidazol-1-ium formate-co-divinylbenzene];
poly [styrene-co-4-vinylbenzenesulfonic acid-co-1-(4-vinylbenzyl)-pyridinium-chloride-co-divinylbenzene];
poly [styrene-co-4-vinylbenzenesulfonic acid-co-1-(4-vinylbenzyl)-pyridinium-bisulfate-co-divinylbenzene];
poly [styrene-co-4-vinylbenzenesulfonic acid-co-1-(4-vinylbenzyl)-pyridinium-acetate-co-divinylbenzene];
poly [styrene-co-4-vinylbenzenesulfonic acid-co-1-(4-vinylbenzyl)-pyridinium-nitrate-co-divinylbenzene];
poly[styrene-co-4-vinylbenzenesulfonic acid-co-1-(4-vinylbenzyl)-pyridinium-chloride-co-3-methyl-1-(4-vinylbenzyl)-3H-imidazol-1-ium bisulfate-co-divinylbenzene];
poly[styrene-co-4-vinylbenzenesulfonic acid-co-1-(4-vinylbenzyl)-pyridinium-bromide-co-3-methyl-1-(4-vinylbenzyl)-3H-imidazol-1-ium bisulfate-co-divinylbenzene];
poly[styrene-co-4-vinylbenzenesulfonic acid-co-1-(4-vinylbenzyl)-pyridinium-iodide-co-3-methyl-1-(4-vinylbenzyl)-3H-imidazol-1-ium bisulfate-co-divinylbenzene];
poly[styrene-co-4-vinylbenzenesulfonic acid-co-1-(4-vinylbenzyl)-pyridinium-bisulfate-co-3-methyl-1-(4-vinylbenzyl)-3H-imidazol-1-ium bisulfate-co-divinylbenzene];
poly[styrene-co-4-vinylbenzenesulfonic acid-co-1-(4-vinylbenzyl)-pyridinium-acetate-co-3-methyl-1-(4-vinylbenzyl)-3H-imidazol-1-ium bisulfate-co-divinylbenzene];
poly[styrene-co-4-vinylbenzenesulfonic acid-co-4-methyl-4-(4-vinylbenzyl)-morpholin-4-ium chloride-co-divinylbenzene];
poly[styrene-co-4-vinylbenzenesulfonic acid-co-4-methyl-4-(4-vinylbenzyl)-morpholin-4-ium bisulfate-co-divinylbenzene];
poly[styrene-co-4-vinylbenzenesulfonic acid-co-4-methyl-4-(4-vinylbenzyl)-morpholin-4-ium acetate-co-divinylbenzene];
poly[styrene-co-4-vinylbenzenesulfonic acid-co-4-methyl-4-(4-vinylbenzyl)-morpholin-4-ium formate-co-divinylbenzene];
poly[styrene-co-4-vinylbenzenesulfonic acid-co-triphenyl-(4-vinylbenzyl)-phosphonium chloride-co-divinylbenzene];
poly[styrene-co-4-vinylbenzenesulfonic acid-co-triphenyl-(4-vinylbenzyl)-phosphonium bisulfate-co-divinylbenzene];
poly[styrene-co-4-vinylbenzenesulfonic acid-co-triphenyl-(4-vinylbenzyl)-phosphonium acetate-co-divinylbenzene];
poly[styrene-co-4-vinylbenzenesulfonic acid-co-1-methyl-1-(4-vinylbenzyl)-piperidin-1-ium chloride-co-divinylbenzene];
poly[styrene-co-4-vinylbenzenesulfonic acid-co-1-methyl-1-(4-vinylbenzyl)-piperidin-1-ium bisulfate-co-divinylbenzene];
poly[styrene-co-4-vinylbenzenesulfonic acid-co-1-methyl-1-(4-vinylbenzyl)-piperidin-1-ium acetate-co-divinylbenzene];
poly[styrene-co-4-vinylbenzenesulfonic acid-co-4-(4-vinylbenzyl)-morpholine-4-oxide-co-divinyl benzene];
poly[styrene-co-4-vinylbenzenesulfonic acid-co-triethyl-(4-vinylbenzyl)-ammonium chloride-co-divinylbenzene];
poly[styrene-co-4-vinylbenzenesulfonic acid-co-triethyl-(4-vinylbenzyl)-ammonium bisulfate-co-divinylbenzene];
poly[styrene-co-4-vinylbenzenesulfonic acid-co-triethyl-(4-vinylbenzyl)-ammonium acetate-co-divinylbenzene];
poly[styrene-co-3-methyl-1-(4-vinylbenzyl)-3H-imidazol-1-ium chloride-co-4-boronyl-1-(4-vinylbenzyl)-pyridinium chloride-co-divinylbenzene];
poly[styrene-co-3-methyl-1-(4-vinylbenzyl)-3H-imidazol-1-ium chloride-co-1-(4-vinylphenyl)methylphosphonic acid-co-divinylbenzene];
poly[styrene-co-3-methyl-1-(4-vinylbenzyl)-3H-imidazol-1-ium bisulfate-co-1-(4-vinylphenyl)methylphosphonic acid-co-divinylbenzene];
poly[styrene-co-3-methyl-1-(4-vinylbenzyl)-3H-imidazol-1-ium acetate-co-1-(4-vinylphenyl)methylphosphonic acid-co-divinylbenzene];
poly[styrene-co-3-methyl-1-(4-vinylbenzyl)-3H-imidazol-1-ium nitrate-co-1-(4-vinylphenyl)methylphosphonic acid-co-divinylbenzene];
poly[styrene-co-4-vinylbenzenesulfonic acid-co-vinylbenzylchloride-co-1-methyl-2-vinyl-pyridinium chloride-co-divinylbenzene];
poly[styrene-co-4-vinylbenzenesulfonic acid-co-vinylbenzylchloride-co-1-methyl-2-vinyl-pyridinium bisulfate-co-divinylbenzene];
poly[styrene-co-4-vinylbenzenesulfonic acid-co-vinylbenzylchloride-co-1-methyl-2-vinyl-pyridinium acetate-co-divinylbenzene];
poly[styrene-co-4-vinylbenzenesulfonic acid-co-4-(4-vinylbenzyl)-morpholine-4-oxide-co-divinyl benzene];
poly [styrene-co-4-vinylphenylphosphonic acid-co-3-methyl-1-(4-vinylbenzyl)-3H-imidazol-1-ium chloride-co-divinylbenzene];
poly [styrene-co-4-vinylphenylphosphonic acid-co-3-methyl-1-(4-vinylbenzyl)-3H-imidazol-1-ium bisulfate-co-divinylbenzene];
poly [styrene-co-4-vinylphenylphosphonic acid-co-3-methyl-1-(4-vinylbenzyl)-3H-imidazol-1-ium acetate-co-divinylbenzene];
poly[styrene-co-3-carboxymethyl-1-(4-vinylbenzyl)-3H-imidazol-1-ium chloride-co-divinylbenzene];
poly[styrene-co-3-carboxymethyl-1-(4-vinylbenzyl)-3H-imidazol-1-ium bisulfate-co-divinylbenzene];
poly[styrene-co-3-carboxymethyl-1-(4-vinylbenzyl)-3H-imidazol-1-ium acetate-co-divinylbenzene];
poly[styrene-co-5-(4-vinylbenzylamino)-isophthalic acid-co-3-methyl-1-(4-vinylbenzyl)-3H-imidazol-1-ium chloride-co-divinylbenzene];
poly[styrene-co-5-(4-vinylbenzylamino)-isophthalic acid-co-3-methyl-1-(4-vinylbenzyl)-3H-imidazol-1-ium bisulfate-co-divinylbenzene];
poly[styrene-co-5-(4-vinylbenzylamino)-isophthalic acid-co-3-methyl-1-(4-vinylbenzyl)-3H-imidazol-1-ium acetate-co-divinylbenzene];
poly[styrene-co-(4-vinylbenzylamino)-acetic acid-co-3-methyl-1-(4-vinylbenzyl)-3H-imidazol-1-ium chloride-co-divinylbenzene];
poly[styrene-co-(4-vinylbenzylamino)-acetic acid-co-3-methyl-1-(4-vinylbenzyl)-3H-imidazol-1-ium bisulfate-co-divinylbenzene];
poly[styrene-co-(4-vinylbenzylamino)-acetic acid-co-3-methyl-1-(4-vinylbenzyl)-3H-imidazol-1-ium acetate-co-divinylbenzene];
poly(styrene-co-4-vinylbenzenesulfonic acid-co-vinylbenzylmethylimidazolium chloride-co-vinylbenzylmethylmorpholinium chloride-co-vinylbenzyltriphenyl phosphonium chloride-co-divinylbenzene);
poly(styrene-co-4-vinylbenzenephosphonic acid-co-vinylbenzylmethylimidazolium chloride-co-vinylbenzylmethylmorpholinium chloride-co-vinylbenzyltriphenyl phosphonium chloride-co-divinylbenzene);
poly(styrene-co-4-vinylbenzenesulfonic acid-co-vinylbenzylmethylimidazolium bisulfate-co-vinylbenzylmethylmorpholinium bisulfate-co-vinylbenzyltriphenyl phosphonium bisulfate-co-divinylbenzene);
poly(styrene-co-4-vinylbenzenephosphonic acid-co-vinylbenzylmethylimidazolium bisulfate-co-vinylbenzylmethylmorpholinium bisulfate-co-vinylbenzyltriphenyl phosphonium bisulfate-co-divinylbenzene);
poly(styrene-co-4-vinylbenzenesulfonic acid-co-vinylbenzylmethylimidazolium acetate-co-vinylbenzylmethylmorpholinium acetate-co-vinylbenzyltriphenyl phosphonium acetate-co-divinylbenzene);
poly(styrene-co-4-vinylbenzenephosphonic acid-co-vinylbenzylmethylimidazolium acetate-co-vinylbenzylmethylmorpholinium acetate-co-vinylbenzyltriphenyl phosphonium acetate-co-divinylbenzene);
poly(styrene-co-4-vinylbenzenesulfonic acid-co-vinylbenzylmethylmorpholinium chloride-co-vinylbenzyltriphenylphosphonium chloride-co-divinylbenzene);
poly(styrene-co-4-vinylbenzenephosphonic acid-co-vinylbenzylmethylmorpholinium chloride-co-vinylbenzyltriphenylphosphonium chloride-co-divinylbenzene);
poly(styrene-co-4-vinylbenzenesulfonic acid-co-vinylbenzylmethylmorpholinium bisulfate-co-vinylbenzyltriphenylphosphonium bisulfate-co-divinylbenzene);
poly(styrene-co-4-vinylbenzenephosphonic acid-co-vinylbenzylmethylmorpholinium bisulfate-co-vinylbenzyltriphenylphosphonium bisulfate-co-divinylbenzene);
poly(styrene-co-4-vinylbenzenesulfonic acid-co-vinylbenzylmethylmorpholinium acetate-co-vinylbenzyltriphenylphosphonium bisulfate-co-divinylbenzene);
poly(styrene-co-4-vinylbenzenephosphonic acid-co-vinylbenzylmethylmorpholinium acetate-co-vinylbenzyltriphenylphosphonium bisulfate-co-divinylbenzene) poly(styrene-co-4-vinylbenzenesulfonic acid-co-vinylmethylimidazolium chloride-co-divinylbenzene);
poly(styrene-co-4-vinylbenzenesulfonic acid-co-vinylmethylimidazolium bisulfate-co-divinylbenzene);
poly(styrene-co-4-vinylbenzenesulfonic acid-co-vinylmethylimidazolium acetate-co-divinylbenzene);
poly(styrene-co-4-vinylbenzenesulfonic acid-co-vinylmethylimidazolium nitrate-co-divinylbenzene);
poly(styrene-co-4-vinylbenzenephosphonic acid-co-vinylmethylimidazolium chloride-co-divinylbenzene);
poly(styrene-co-4-vinylbenzenephosphonic acid-co-vinylmethylimidazolium bisulfate-co-divinylbenzene);
poly(styrene-co-4-vinylbenzenephosphonic acid-co-vinylmethylimidazolium acetate-co-divinylbenzene);
poly(styrene-co-4-vinylbenzenesulfonic acid-co-vinylbenzyltriphenylphosphonium chloride-co-divinylbenzene);
poly(styrene-co-4-vinylbenzenesulfonic acid-co-vinylbenzyltriphenylphosphonium bisulfate-co-divinylbenzene);
poly(styrene-co-4-vinylbenzenesulfonic acid-co-vinylbenzyltriphenylphosphonium acetate-co-divinylbenzene);
poly(styrene-co-4-vinylbenzenephosphonic acid-co-vinylbenzyltriphenylphosphonium chloride-co-divinylbenzene);
poly(styrene-co-4-vinylbenzenephosphonic acid-co-vinylbenzyltriphenylphosphonium bisulfate-co-divinylbenzene);
poly(styrene-co-4-vinylbenzenephosphonic acid-co-vinylbenzyltriphenylphosphonium acetate-co-divinylbenzene);
poly(styrene-co-4-vinylbenzenesulfonic acid-co-vinylbenzylmethylimidazolium chloride-co-divinylbenzene);
poly(styrene-co-4-vinylbenzenesulfonic acid-co-vinylbenzylmethylimidazolium bisulfate-co-divinylbenzene);
poly(styrene-co-4-vinylbenzenesulfonic acid-co-vinylbenzylmethylimidazolium acetate-co-divinylbenzene);
poly(styrene-co-4-vinylbenzenephosphonic acid-co-vinylbenzylmethylimidazolium chloride-co-divinylbenzene);
poly(styrene-co-4-vinylbenzenephosphonic acid-co-vinylbenzylmethylimidazolium bisulfate-co-divinylbenzene);
poly(styrene-co-4-vinylbenzenephosphonic acid-co-vinylbenzylmethylimidazolium acetate-co-divinylbenzene);
poly(styrene-co-4-vinylbenzenesulfonic acid-co-vinylbenzyltriphenylphosphonium chloride-co-divinylbenzene);
poly(styrene-co-4-vinylbenzenesulfonic acid-co-vinylbenzyltriphenylphosphonium bisulfate-co-divinylbenzene);
poly(styrene-co-4-vinylbenzenesulfonic acid-co-vinylbenzyltriphenylphosphonium acetate-co-divinylbenzene);
poly(styrene-co-4-vinylbenzenephosphonic acid-co-vinylbenzyltriphenylphosphonium chloride-co-divinylbenzene);
poly(styrene-co-4-vinylbenzenephosphonic acid-co-vinylbenzyltriphenylphosphonium bisulfate-co-divinylbenzene);
poly(styrene-co-4-vinylbenzenephosphonic acid-co-vinylbenzyltriphenylphosphonium acetate-co-divinylbenzene);
poly(butyl-vinylimidazolium chloride-co-butylimidazolium bisulfate-co-4-vinylbenzenesulfonic acid);
poly(butyl-vinylimidazolium bisulfate-co-butylimidazolium bisulfate-co-4-vinylbenzenesulfonic acid);
poly(benzyl alcohol-co-4-vinylbenzylalcohol sulfonic acid-co-vinylbenzyltriphenylphosphonium chloride-co-divinylbenzyl alcohol); and
poly(benzyl alcohol-co-4-vinylbenzylalcohol sulfonic acid-co-vinylbenzyltriphenylphosphonium bisulfate-co-divinylbenzyl alcohol).

In some embodiments, the solid-supported catalyst is selected from:

amorphous carbon-supported pyrrolium chloride sulfonic acid;

amorphous carbon-supported imidazolium chloride sulfonic acid;

amorphous carbon-supported pyrazolium chloride sulfonic acid;

amorphous carbon-supported oxazolium chloride sulfonic acid;

amorphous carbon-supported thiazolium chloride sulfonic acid;

amorphous carbon-supported pyridinium chloride sulfonic acid;

amorphous carbon-supported pyrimidinium chloride sulfonic acid;

amorphous carbon-supported pyrazinium chloride sulfonic acid;

amorphous carbon-supported pyridazinium chloride sulfonic acid;

amorphous carbon-supported thiazinium chloride sulfonic acid;

amorphous carbon-supported morpholinium chloride sulfonic acid;

amorphous carbon-supported piperidinium chloride sulfonic acid;

amorphous carbon-supported piperizinium chloride sulfonic acid;

amorphous carbon-supported pyrollizinium chloride sulfonic acid;

amorphous carbon-supported triphenyl phosphonium chloride sulfonic acid;

amorphous carbon-supported trimethyl phosphonium chloride sulfonic acid;

amorphous carbon-supported triethyl phosphonium chloride sulfonic acid;

amorphous carbon-supported tripropyl phosphonium chloride sulfonic acid;

amorphous carbon-supported tributyl phosphonium chloride sulfonic acid;

amorphous carbon-supported trifluoro phosphonium chloride sulfonic acid;

amorphous carbon-supported pyrrolium bromide sulfonic acid;

amorphous carbon-supported imidazolium bromide sulfonic acid;

amorphous carbon-supported pyrazolium bromide sulfonic acid;

amorphous carbon-supported oxazolium bromide sulfonic acid;

amorphous carbon-supported thiazolium bromide sulfonic acid;

amorphous carbon-supported pyridinium bromide sulfonic acid;

amorphous carbon-supported pyrimidinium bromide sulfonic acid;

amorphous carbon-supported pyrazinium bromide sulfonic acid;

amorphous carbon-supported pyridazinium bromide sulfonic acid;

amorphous carbon-supported thiazinium bromide sulfonic acid;

amorphous carbon-supported morpholinium bromide sulfonic acid;

amorphous carbon-supported piperidinium bromide sulfonic acid;

amorphous carbon-supported piperizinium bromide sulfonic acid;

amorphous carbon-supported pyrollizinium bromide sulfonic acid;

amorphous carbon-supported triphenyl phosphonium bromide sulfonic acid;

amorphous carbon-supported trimethyl phosphonium bromide sulfonic acid;

amorphous carbon-supported triethyl phosphonium bromide sulfonic acid;

amorphous carbon-supported tripropyl phosphonium bromide sulfonic acid;

amorphous carbon-supported tributyl phosphonium bromide sulfonic acid;

amorphous carbon-supported trifluoro phosphonium bromide sulfonic acid;

amorphous carbon-supported pyrrolium bisulfate sulfonic acid;

amorphous carbon-supported imidazolium bisulfate sulfonic acid;

amorphous carbon-supported pyrazolium bisulfate sulfonic acid;

amorphous carbon-supported oxazolium bisulfate sulfonic acid;

amorphous carbon-supported thiazolium bisulfate sulfonic acid;

amorphous carbon-supported pyridinium bisulfate sulfonic acid;

amorphous carbon-supported pyrimidinium bisulfate sulfonic acid;

amorphous carbon-supported pyrazinium bisulfate sulfonic acid;

amorphous carbon-supported pyridazinium bisulfate sulfonic acid;

amorphous carbon-supported thiazinium bisulfate sulfonic acid;

amorphous carbon-supported morpholinium bisulfate sulfonic acid;

amorphous carbon-supported piperidinium bisulfate sulfonic acid;

amorphous carbon-supported piperizinium bisulfate sulfonic acid;

amorphous carbon-supported pyrollizinium bisulfate sulfonic acid;

amorphous carbon-supported triphenyl phosphonium bisulfate sulfonic acid;

amorphous carbon-supported trimethyl phosphonium bisulfate sulfonic acid;

amorphous carbon-supported triethyl phosphonium bisulfate sulfonic acid;

amorphous carbon-supported tripropyl phosphonium bisulfate sulfonic acid;

amorphous carbon-supported tributyl phosphonium bisulfate sulfonic acid;

amorphous carbon-supported trifluoro phosphonium bisulfate sulfonic acid;

amorphous carbon-supported pyrrolium formate sulfonic acid;

amorphous carbon-supported imidazolium formate sulfonic acid;

amorphous carbon-supported pyrazolium formate sulfonic acid;

amorphous carbon-supported oxazolium formate sulfonic acid;

amorphous carbon-supported thiazolium formate sulfonic acid;

amorphous carbon-supported pyridinium formate sulfonic acid;

amorphous carbon-supported pyrimidinium formate sulfonic acid;

amorphous carbon-supported pyrazinium formate sulfonic acid;

amorphous carbon-supported pyridazinium formate sulfonic acid;

amorphous carbon-supported thiazinium formate sulfonic acid;

amorphous carbon supported morpholinium formate sulfonic acid;

amorphous carbon-supported piperidinium formate sulfonic acid;

amorphous carbon-supported piperizinium formate sulfonic acid;

amorphous carbon-supported pyrollizinium formate sulfonic acid;

amorphous carbon-supported triphenyl phosphonium formate sulfonic acid;

amorphous carbon-supported trimethyl phosphonium formate sulfonic acid;

amorphous carbon-supported triethyl phosphonium formate sulfonic acid;

amorphous carbon-supported tripropyl phosphonium formate sulfonic acid;

amorphous carbon-supported tributyl phosphonium formate sulfonic acid;

amorphous carbon-supported trifluoro phosphonium formate sulfonic acid;

amorphous carbon-supported pyrrolium acetate sulfonic acid;

amorphous carbon-supported imidazolium acetate sulfonic acid;

amorphous carbon-supported pyrazolium acetate sulfonic acid;

amorphous carbon-supported oxazolium acetate sulfonic acid;

amorphous carbon-supported thiazolium acetate sulfonic acid;

amorphous carbon-supported pyridinium acetate sulfonic acid;

amorphous carbon-supported pyrimidinium acetate sulfonic acid;

amorphous carbon-supported pyrazinium acetate sulfonic acid;

amorphous carbon-supported pyridazinium acetate sulfonic acid;

amorphous carbon-supported thiazinium acetate sulfonic acid;

amorphous carbon-supported morpholinium acetate sulfonic acid;

amorphous carbon-supported piperidinium acetate sulfonic acid;

amorphous carbon-supported piperizinium acetate sulfonic acid;

amorphous carbon-supported pyrollizinium acetate sulfonic acid;

amorphous carbon-supported triphenyl phosphonium acetate sulfonic acid;

amorphous carbon-supported trimethyl phosphonium acetate sulfonic acid;

amorphous carbon-supported triethyl phosphonium acetate sulfonic acid;

amorphous carbon-supported tripropyl phosphonium acetate sulfonic acid;

amorphous carbon-supported tributyl phosphonium acetate sulfonic acid;

amorphous carbon-supported trifluoro phosphonium acetate sulfonic acid;

amorphous carbon-supported pyrrolium chloride phosphonic acid;

amorphous carbon-supported imidazolium chloride phosphonic acid;

amorphous carbon-supported pyrazolium chloride phosphonic acid;

amorphous carbon-supported oxazolium chloride phosphonic acid;

amorphous carbon-supported thiazolium chloride phosphonic acid;

amorphous carbon-supported pyridinium chloride phosphonic acid;

amorphous carbon-supported pyrimidinium chloride phosphonic acid;

amorphous carbon-supported pyrazinium chloride phosphonic acid;

amorphous carbon-supported pyridazinium chloride phosphonic acid;

amorphous carbon-supported thiazinium chloride phosphonic acid;

amorphous carbon-supported morpholinium chloride phosphonic acid;

amorphous carbon-supported piperidinium chloride phosphonic acid;

amorphous carbon-supported piperizinium chloride phosphonic acid;

amorphous carbon-supported pyrollizinium chloride phosphonic acid;

amorphous carbon-supported triphenyl phosphonium chloride phosphonic acid;

amorphous carbon-supported trimethyl phosphonium chloride phosphonic acid;

amorphous carbon-supported triethyl phosphonium chloride phosphonic acid;

amorphous carbon-supported tripropyl phosphonium chloride phosphonic acid;

amorphous carbon-supported tributyl phosphonium chloride phosphonic acid;

amorphous carbon-supported trifluoro phosphonium chloride phosphonic acid;

amorphous carbon-supported pyrrolium bromide phosphonic acid;

amorphous carbon-supported imidazolium bromide phosphonic acid;

amorphous carbon-supported pyrazolium bromide phosphonic acid;

amorphous carbon-supported oxazolium bromide phosphonic acid;

amorphous carbon-supported thiazolium bromide phosphonic acid;

amorphous carbon-supported pyridinium bromide phosphonic acid;

amorphous carbon-supported pyrimidinium bromide phosphonic acid;

amorphous carbon-supported pyrazinium bromide phosphonic acid;

amorphous carbon-supported pyridazinium bromide phosphonic acid;

amorphous carbon-supported thiazinium bromide phosphonic acid;

amorphous carbon-supported morpholinium bromide phosphonic acid;

amorphous carbon-supported piperidinium bromide phosphonic acid;

amorphous carbon-supported piperizinium bromide phosphonic acid;

amorphous carbon-supported pyrollizinium bromide phosphonic acid;

amorphous carbon-supported triphenyl phosphonium bromide phosphonic acid;

amorphous carbon-supported trimethyl phosphonium bromide phosphonic acid;

amorphous carbon-supported triethyl phosphonium bromide phosphonic acid;

amorphous carbon-supported tripropyl phosphonium bromide phosphonic acid;

amorphous carbon-supported tributyl phosphonium bromide phosphonic acid;

amorphous carbon-supported trifluoro phosphonium bromide phosphonic acid;

amorphous carbon-supported pyrrolium bisulfate phosphonic acid;

amorphous carbon-supported imidazolium bisulfate phosphonic acid;

amorphous carbon-supported pyrazolium bisulfate phosphonic acid;

amorphous carbon-supported oxazolium bisulfate phosphonic acid;

amorphous carbon-supported thiazolium bisulfate phosphonic acid;

amorphous carbon-supported pyridinium bisulfate phosphonic acid;

amorphous carbon-supported pyrimidinium bisulfate phosphonic acid;

amorphous carbon-supported pyrazinium bisulfate phosphonic acid;

amorphous carbon-supported pyridazinium bisulfate phosphonic acid;

amorphous carbon-supported thiazinium bisulfate phosphonic acid;

amorphous carbon-supported morpholinium bisulfate phosphonic acid;

amorphous carbon-supported piperidinium bisulfate phosphonic acid;

amorphous carbon-supported piperizinium bisulfate phosphonic acid;

amorphous carbon-supported pyrollizinium bisulfate phosphonic acid;

amorphous carbon-supported triphenyl phosphonium bisulfate phosphonic acid;

amorphous carbon-supported trimethyl phosphonium bisulfate phosphonic acid;

amorphous carbon-supported triethyl phosphonium bisulfate phosphonic acid;

amorphous carbon-supported tripropyl phosphonium bisulfate phosphonic acid;

amorphous carbon-supported tributyl phosphonium bisulfate phosphonic acid;

amorphous carbon-supported trifluoro phosphonium bisulfate phosphonic acid;

amorphous carbon-supported pyrrolium formate phosphonic acid;

amorphous carbon-supported imidazolium formate phosphonic acid;

amorphous carbon-supported pyrazolium formate phosphonic acid;

amorphous carbon-supported oxazolium formate phosphonic acid;

amorphous carbon-supported thiazolium formate phosphonic acid;

amorphous carbon-supported pyridinium formate phosphonic acid;

amorphous carbon-supported pyrimidinium formate phosphonic acid;

amorphous carbon-supported pyrazinium formate phosphonic acid;

amorphous carbon-supported pyridazinium formate phosphonic acid;

amorphous carbon-supported thiazinium formate phosphonic acid;

amorphous carbon-supported morpholinium formate phosphonic acid;

amorphous carbon-supported piperidinium formate phosphonic acid;

amorphous carbon-supported piperizinium formate phosphonic acid;

amorphous carbon-supported pyrollizinium formate phosphonic acid;

amorphous carbon-supported triphenyl phosphonium formate phosphonic acid;

amorphous carbon-supported trimethyl phosphonium formate phosphonic acid;

amorphous carbon-supported triethyl phosphonium formate phosphonic acid;

amorphous carbon-supported tripropyl phosphonium formate phosphonic acid;

amorphous carbon-supported tributyl phosphonium formate phosphonic acid;

amorphous carbon-supported trifluoro phosphonium formate phosphonic acid;

amorphous carbon-supported pyrrolium acetate phosphonic acid;

amorphous carbon-supported imidazolium acetate phosphonic acid;

amorphous carbon-supported pyrazolium acetate phosphonic acid;

amorphous carbon-supported oxazolium acetate phosphonic acid;

amorphous carbon-supported thiazolium acetate phosphonic acid;

amorphous carbon-supported pyridinium acetate phosphonic acid;

amorphous carbon-supported pyrimidinium acetate phosphonic acid;

amorphous carbon-supported pyrazinium acetate phosphonic acid;

amorphous carbon-supported pyridazinium acetate phosphonic acid;

amorphous carbon-supported thiazinium acetate phosphonic acid;

amorphous carbon-supported morpholinium acetate phosphonic acid;

amorphous carbon-supported piperidinium acetate phosphonic acid;

amorphous carbon-supported piperizinium acetate phosphonic acid;

amorphous carbon-supported pyrollizinium acetate phosphonic acid;

amorphous carbon-supported triphenyl phosphonium acetate phosphonic acid;

amorphous carbon-supported trimethyl phosphonium acetate phosphonic acid;

amorphous carbon-supported triethyl phosphonium acetate phosphonic acid;

amorphous carbon-supported tripropyl phosphonium acetate phosphonic acid;

amorphous carbon-supported tributyl phosphonium acetate phosphonic acid;

amorphous carbon-supported trifluoro phosphonium acetate phosphonic acid;

amorphous carbon-supported ethanoyl-triphosphonium sulfonic acid;

amorphous carbon-supported ethanoyl-methylmorpholinium sulfonic acid; and

amorphous carbon-supported ethanoyl-imidazolium sulfonic acid.

In other embodiments, the solid-supported catalyst is selected from:

activated carbon-supported pyrrolium chloride sulfonic acid;

activated carbon-supported imidazolium chloride sulfonic acid;

activated carbon-supported pyrazolium chloride sulfonic acid;

activated carbon-supported oxazolium chloride sulfonic acid;

activated carbon-supported thiazolium chloride sulfonic acid;

activated carbon-supported pyridinium chloride sulfonic acid;

activated carbon-supported pyrimidinium chloride sulfonic acid;

activated carbon-supported pyrazinium chloride sulfonic acid;

activated carbon-supported pyridazinium chloride sulfonic acid;

activated carbon-supported thiazinium chloride sulfonic acid;

activated carbon-supported morpholinium chloride sulfonic acid;

activated carbon-supported piperidinium chloride sulfonic acid;

activated carbon-supported piperizinium chloride sulfonic acid;

activated carbon-supported pyrollizinium chloride sulfonic acid;

activated carbon-supported triphenyl phosphonium chloride sulfonic acid;

activated carbon-supported trimethyl phosphonium chloride sulfonic acid;

activated carbon-supported triethyl phosphonium chloride sulfonic acid;

activated carbon-supported tripropyl phosphonium chloride sulfonic acid;

activated carbon-supported tributyl phosphonium chloride sulfonic acid;

activated carbon-supported trifluoro phosphonium chloride sulfonic acid;

activated carbon-supported pyrrolium bromide sulfonic acid;

activated carbon-supported imidazolium bromide sulfonic acid;

activated carbon-supported pyrazolium bromide sulfonic acid;

activated carbon-supported oxazolium bromide sulfonic acid;

activated carbon-supported thiazolium bromide sulfonic acid;

activated carbon-supported pyridinium bromide sulfonic acid;

activated carbon-supported pyrimidinium bromide sulfonic acid;

activated carbon-supported pyrazinium bromide sulfonic acid;

activated carbon-supported pyridazinium bromide sulfonic acid;

activated carbon-supported thiazinium bromide sulfonic acid;

activated carbon-supported morpholinium bromide sulfonic acid;

activated carbon-supported piperidinium bromide sulfonic acid;

activated carbon-supported piperizinium bromide sulfonic acid;

activated carbon-supported pyrollizinium bromide sulfonic acid;

activated carbon-supported triphenyl phosphonium bromide sulfonic acid;

activated carbon-supported trimethyl phosphonium bromide sulfonic acid;

activated carbon-supported triethyl phosphonium bromide sulfonic acid;

activated carbon-supported tripropyl phosphonium bromide sulfonic acid;

activated carbon-supported tributyl phosphonium bromide sulfonic acid;

activated carbon-supported trifluoro phosphonium bromide sulfonic acid;

activated carbon-supported pyrrolium bisulfate sulfonic acid;

activated carbon-supported imidazolium bisulfate sulfonic acid;

activated carbon-supported pyrazolium bisulfate sulfonic acid;

activated carbon-supported oxazolium bisulfate sulfonic acid;

activated carbon-supported thiazolium bisulfate sulfonic acid;

activated carbon-supported pyridinium bisulfate sulfonic acid;

activated carbon-supported pyrimidinium bisulfate sulfonic acid;

activated carbon-supported pyrazinium bisulfate sulfonic acid;

activated carbon-supported pyridazinium bisulfate sulfonic acid;

activated carbon-supported thiazinium bisulfate sulfonic acid;

activated carbon-supported morpholinium bisulfate sulfonic acid;

activated carbon-supported piperidinium bisulfate sulfonic acid;

activated carbon-supported piperizinium bisulfate sulfonic acid;

activated carbon-supported pyrollizinium bisulfate sulfonic acid;

activated carbon-supported triphenyl phosphonium bisulfate sulfonic acid;

activated carbon-supported trimethyl phosphonium bisulfate sulfonic acid;

activated carbon-supported triethyl phosphonium bisulfate sulfonic acid;

activated carbon-supported tripropyl phosphonium bisulfate sulfonic acid;

activated carbon-supported tributyl phosphonium bisulfate sulfonic acid;

activated carbon-supported trifluoro phosphonium bisulfate sulfonic acid;

activated carbon-supported pyrrolium formate sulfonic acid;

activated carbon-supported imidazolium formate sulfonic acid;

activated carbon-supported pyrazolium formate sulfonic acid;

activated carbon-supported oxazolium formate sulfonic acid;

activated carbon-supported thiazolium formate sulfonic acid;

activated carbon-supported pyridinium formate sulfonic acid;

activated carbon-supported pyrimidinium formate sulfonic acid;

activated carbon-supported pyrazinium formate sulfonic acid;

activated carbon-supported pyridazinium formate sulfonic acid;

activated carbon-supported thiazinium formate sulfonic acid;

activated carbon supported morpholinium formate sulfonic acid;

activated carbon-supported piperidinium formate sulfonic acid;

activated carbon-supported piperizinium formate sulfonic acid;

activated carbon-supported pyrollizinium formate sulfonic acid;

activated carbon-supported triphenyl phosphonium formate sulfonic acid;

activated carbon-supported trimethyl phosphonium formate sulfonic acid;

activated carbon-supported triethyl phosphonium formate sulfonic acid;

activated carbon-supported tripropyl phosphonium formate sulfonic acid;

activated carbon-supported tributyl phosphonium formate sulfonic acid;

activated carbon-supported trifluoro phosphonium formate sulfonic acid;

activated carbon-supported pyrrolium acetate sulfonic acid;

activated carbon-supported imidazolium acetate sulfonic acid;

activated carbon-supported pyrazolium acetate sulfonic acid;

activated carbon-supported oxazolium acetate sulfonic acid;

activated carbon-supported thiazolium acetate sulfonic acid;

activated carbon-supported pyridinium acetate sulfonic acid;

activated carbon-supported pyrimidinium acetate sulfonic acid;

activated carbon-supported pyrazinium acetate sulfonic acid;

activated carbon-supported pyridazinium acetate sulfonic acid;

activated carbon-supported thiazinium acetate sulfonic acid;

activated carbon-supported morpholinium acetate sulfonic acid;

activated carbon-supported piperidinium acetate sulfonic acid;

activated carbon-supported piperizinium acetate sulfonic acid;

activated carbon-supported pyrollizinium acetate sulfonic acid;

activated carbon-supported triphenyl phosphonium acetate sulfonic acid;

activated carbon-supported trimethyl phosphonium acetate sulfonic acid;

activated carbon-supported triethyl phosphonium acetate sulfonic acid;

activated carbon-supported tripropyl phosphonium acetate sulfonic acid;

activated carbon-supported tributyl phosphonium acetate sulfonic acid;

activated carbon-supported trifluoro phosphonium acetate sulfonic acid;

activated carbon-supported pyrrolium chloride phosphonic acid;

activated carbon-supported imidazolium chloride phosphonic acid;

activated carbon-supported pyrazolium chloride phosphonic acid;

activated carbon-supported oxazolium chloride phosphonic acid;

activated carbon-supported thiazolium chloride phosphonic acid;

activated carbon-supported pyridinium chloride phosphonic acid;

activated carbon-supported pyrimidinium chloride phosphonic acid;

activated carbon-supported pyrazinium chloride phosphonic acid;

activated carbon-supported pyridazinium chloride phosphonic acid;

activated carbon-supported thiazinium chloride phosphonic acid;

activated carbon-supported morpholinium chloride phosphonic acid;

activated carbon-supported piperidinium chloride phosphonic acid;

activated carbon-supported piperizinium chloride phosphonic acid;

activated carbon-supported pyrollizinium chloride phosphonic acid;

activated carbon-supported triphenyl phosphonium chloride phosphonic acid;

activated carbon-supported trimethyl phosphonium chloride phosphonic acid;

activated carbon-supported triethyl phosphonium chloride phosphonic acid;

activated carbon-supported tripropyl phosphonium chloride phosphonic acid;

activated carbon-supported tributyl phosphonium chloride phosphonic acid;

activated carbon-supported trifluoro phosphonium chloride phosphonic acid;

activated carbon-supported pyrrolium bromide phosphonic acid;

activated carbon-supported imidazolium bromide phosphonic acid;

activated carbon-supported pyrazolium bromide phosphonic acid;

activated carbon-supported oxazolium bromide phosphonic acid;

activated carbon-supported thiazolium bromide phosphonic acid;

activated carbon-supported pyridinium bromide phosphonic acid;

activated carbon-supported pyrimidinium bromide phosphonic acid;

activated carbon-supported pyrazinium bromide phosphonic acid;

activated carbon-supported pyridazinium bromide phosphonic acid;

activated carbon-supported thiazinium bromide phosphonic acid;

activated carbon-supported morpholinium bromide phosphonic acid;

activated carbon-supported piperidinium bromide phosphonic acid;

activated carbon-supported piperizinium bromide phosphonic acid;

activated carbon-supported pyrollizinium bromide phosphonic acid;

activated carbon-supported triphenyl phosphonium bromide phosphonic acid;

activated carbon-supported trimethyl phosphonium bromide phosphonic acid;

activated carbon-supported triethyl phosphonium bromide phosphonic acid;

activated carbon-supported tripropyl phosphonium bromide phosphonic acid;

activated carbon-supported tributyl phosphonium bromide phosphonic acid;

activated carbon-supported trifluoro phosphonium bromide phosphonic acid;

activated carbon-supported pyrrolium bisulfate phosphonic acid;

activated carbon-supported imidazolium bisulfate phosphonic acid;

activated carbon-supported pyrazolium bisulfate phosphonic acid;

activated carbon-supported oxazolium bisulfate phosphonic acid;

activated carbon-supported thiazolium bisulfate phosphonic acid;

activated carbon-supported pyridinium bisulfate phosphonic acid;

activated carbon-supported pyrimidinium bisulfate phosphonic acid;

activated carbon-supported pyrazinium bisulfate phosphonic acid;

activated carbon-supported pyridazinium bisulfate phosphonic acid;

activated carbon-supported thiazinium bisulfate phosphonic acid;

activated carbon-supported morpholinium bisulfate phosphonic acid;

activated carbon-supported piperidinium bisulfate phosphonic acid;

activated carbon-supported piperizinium bisulfate phosphonic acid;

activated carbon-supported pyrollizinium bisulfate phosphonic acid;

activated carbon-supported triphenyl phosphonium bisulfate phosphonic acid;

activated carbon-supported trimethyl phosphonium bisulfate phosphonic acid;

activated carbon-supported triethyl phosphonium bisulfate phosphonic acid;

activated carbon-supported tripropyl phosphonium bisulfate phosphonic acid;

activated carbon-supported tributyl phosphonium bisulfate phosphonic acid;

activated carbon-supported trifluoro phosphonium bisulfate phosphonic acid;

activated carbon-supported pyrrolium formate phosphonic acid;

activated carbon-supported imidazolium formate phosphonic acid;

activated carbon-supported pyrazolium formate phosphonic acid;

activated carbon-supported oxazolium formate phosphonic acid;

activated carbon-supported thiazolium formate phosphonic acid;

activated carbon-supported pyridinium formate phosphonic acid;

activated carbon-supported pyrimidinium formate phosphonic acid;

activated carbon-supported pyrazinium formate phosphonic acid;

activated carbon-supported pyridazinium formate phosphonic acid;

activated carbon-supported thiazinium formate phosphonic acid;

activated carbon-supported morpholinium formate phosphonic acid;

activated carbon-supported piperidinium formate phosphonic acid;

activated carbon-supported piperizinium formate phosphonic acid;

activated carbon-supported pyrollizinium formate phosphonic acid;

activated carbon-supported triphenyl phosphonium formate phosphonic acid;

activated carbon-supported trimethyl phosphonium formate phosphonic acid;

activated carbon-supported triethyl phosphonium formate phosphonic acid;

activated carbon-supported tripropyl phosphonium formate phosphonic acid;

activated carbon-supported tributyl phosphonium formate phosphonic acid;

activated carbon-supported trifluoro phosphonium formate phosphonic acid;

activated carbon-supported pyrrolium acetate phosphonic acid;

activated carbon-supported imidazolium acetate phosphonic acid;

activated carbon-supported pyrazolium acetate phosphonic acid;

activated carbon-supported oxazolium acetate phosphonic acid;

activated carbon-supported thiazolium acetate phosphonic acid;

activated carbon-supported pyridinium acetate phosphonic acid;

activated carbon-supported pyrimidinium acetate phosphonic acid;

activated carbon-supported pyrazinium acetate phosphonic acid;

activated carbon-supported pyridazinium acetate phosphonic acid;

activated carbon-supported thiazinium acetate phosphonic acid;

activated carbon-supported morpholinium acetate phosphonic acid;

activated carbon-supported piperidinium acetate phosphonic acid;

activated carbon-supported piperizinium acetate phosphonic acid;

activated carbon-supported pyrollizinium acetate phosphonic acid;

activated carbon-supported triphenyl phosphonium acetate phosphonic acid;

activated carbon-supported trimethyl phosphonium acetate phosphonic acid;

activated carbon-supported triethyl phosphonium acetate phosphonic acid;

activated carbon-supported tripropyl phosphonium acetate phosphonic acid;

activated carbon-supported tributyl phosphonium acetate phosphonic acid;

activated carbon-supported trifluoro phosphonium acetate phosphonic acid;

activated carbon-supported ethanoyl-triphosphonium sulfonic acid;

activated carbon-supported ethanoyl-methylmorpholinium sulfonic acid; and

activated carbon-supported ethanoyl-imidazolium sulfonic acid.

Methods to prepare the polymeric and solid-supported catalysts described herein can be found in WO 2014/031956, which is hereby incorporated herein specifically with respect to paragraphs [0345]-[0380] and [0382]-[0472].

c) Reaction Conditions for Catalytic Oligosaccharide Formation

In some embodiments, the feed sugar and catalyst (e.g., polymeric catalyst or solid-supported catalyst) are allowed to react for at least 1 hour, at least 2 hours, at least 3 hours, at least 4 hours, at least 6 hours, at least 8 hours, at least 16 hours, at least 24 hours, at least 36 hours, or at least 48 hours; or between 1-24 hours, between 2-12 hours, between 3-6 hours, between 1-96 hours, between 12-72 hours, or between 12-48 hours.

In some embodiments, the degree of polymerization of the one or more oligosaccharides produced according to the methods described herein can be regulated by the reaction time. For example, in some embodiments, the degree of polymerization of the one or more oligosaccharides is increased by increasing the reaction time, while in other embodiments, the degree of polymerization of the one or more oligosaccharides is decreased by decreasing the reaction time.

Reaction Temperature

In some embodiments, the reaction temperature is maintained in the range of about 25° C. to about 150° C. In certain embodiments, the temperature is from about 30° C. to about 125° C., about 60° C. to about 120° C., about 80° C. to about 115° C., about 90° C. to about 110° C., about 95° C. to about 105° C., or about 100° C. to 110° C.

Amount of Feed Sugar

The amount of the feed sugar used in the methods described herein relative to the amount solvent used may affect the rate of reaction and yield. The amount of the feed sugar used may be characterized by the dry solids content. In certain embodiments, dry solids content refers to the total solids of a slurry as a percentage on a dry weight basis. In some embodiments, the dry solids content of the feed sugar is between about 5 wt % to about 95 wt %, between about 10 wt % to about 80 wt %, between about 15 to about 75 wt %, or between about 15 to about 50 wt %.

Amount of Catalyst

The amount of the catalyst used in the methods described herein may depend on several factors including, for example, the selection of the type of feed sugar, the concentration of the feed sugar, and the reaction conditions (e.g., temperature, time, and pH). In some embodiments, the weight ratio of the catalyst to the feed sugar is about 0.01 g/g to about 50 g/g, about 0.01 g/g to about 5 g/g, about 0.05 g/g to about 1.0 g/g, about 0.05 g/g to about 0.5 g/g, about 0.05 g/g to about 0.2 g/g, or about 0.1 g/g to about 0.2 g/g.

Solvent

In certain embodiments, the methods of using the catalyst are carried out in an aqueous environment. One suitable aqueous solvent is water, which may be obtained from various sources. Generally, water sources with lower concentrations of ionic species (e.g., salts of sodium, phosphorous, ammonium, or magnesium) are preferable, as such ionic species may reduce effectiveness of the catalyst. In some embodiments where the aqueous solvent is water, the water has a resistivity of at least 0.1 megaohm-centimeters, of at least 1 megaohm-centimeters, of at least 2 megaohm-centimeters, of at least 5 megaohm-centimeters, or of at least 10 megaohm-centimeters.

Water Content

Moreover, as the dehydration reaction of the methods progresses, water is produced with each coupling of the one or more sugars. In certain embodiments, the methods described herein may further include monitoring the amount of water present in the reaction mixture and/or the ratio of water to sugar or catalyst over a period of time. In some embodiments, the method further includes removing at least a portion of water produced in the reaction mixture (e.g., by removing at least about any of 10%, 20%, 30%, 40%, 50%, 60%, 70%, 80%, 90%, 95%, 97%, 99%, or 100%, such as by vacuum distillation). It should be understood, however, that the amount of water to sugar may be adjusted based on the reaction conditions and specific catalyst used.

Any method known in the art may be used to remove water in the reaction mixture, including, for example, by vacuum filtration, vacuum distillation, heating, and/or evaporation. In some embodiments, the method comprises including water in the reaction mixture.

In some aspects, provided herein are methods of producing an oligosaccharide composition, by: combining a feed sugar and a catalyst having acidic and ionic moieties to form a reaction mixture, wherein water is produced in the reaction mixture; and removing at least a portion of the water produced in the reaction mixture. In certain variations, at least a portion of water is removed to maintain a water content in the reaction mixture of less than 99%, less than 90%, less than 80%, less than 70%, less than 60%, less than 50%, less than 40%, less than 30%, less than 20%, less than 10%, less than 5%, or less than 1% by weight.

In some embodiments, the degree of polymerization of the one or more oligosaccharides produced according to the methods described herein can be regulated by adjusting or controlling the concentration of water present in the reaction mixture. For example, in some embodiments, the degree of polymerization of the one or more oligosaccharides is increased by decreasing the water concentration, while in other embodiments, the degree of polymerization of the one or more oligosaccharides is decreased by increasing the water concentration. In some embodiments, the water content of the reaction is adjusted during the reaction to regulate the degree of polymerization of the one or more oligosaccharides produced.

Batch Versus Continuous Processing

Generally, the catalyst and the feed sugar are introduced into an interior chamber of a reactor, either concurrently or sequentially. The reaction can be performed in a batch process or a continuous process. For example, in one embodiment, method is performed in a batch process, where the contents of the reactor are continuously mixed or blended, and all or a substantial amount of the products of the reaction are removed. In one variation, the method is performed in a batch process, where the contents of the reactor are initially intermingled or mixed but no further physical mixing is performed. In another variation, the method is performed in a batch process, wherein once further mixing of the contents, or periodic mixing of the contents of the reactor, is performed (e.g., at one or more times per hour), all or a substantial amount of the products of the reaction are removed after a certain period of time.

In some embodiments, the method is repeated in a sequential batch process, wherein at least a portion of the catalyst is separated from at least a portion of the oligosaccharide composition produced (e.g., as described in more detail infra) and is recycled by further contacting additional feed sugar.

For example, in one aspect, provided is a method for producing an oligosaccharide composition, by:

a) combining feed sugar with a catalyst to form a reaction mixture;
wherein the catalyst comprises acidic monomers and ionic monomers connected to form a polymeric backbone, or
wherein the catalyst comprises a solid support, acidic moieties attached to the solid support, and ionic moieties attached to the solid support; and

b) producing an oligosaccharide composition from at least a portion of the reaction mixture;

c) separating the oligosaccharide composition from the catalyst;

d) combining additional feed sugar with the separated catalyst to form additional reaction mixture; and

e) producing additional oligosaccharide composition from at least a portion of the additional reaction mixture.

In some of embodiments wherein the method is performed in a batch process, the catalyst is recycled (e.g., steps (c)-(e) above are repeated) at least 1, at least 2, at least 3, at least 4, at least 5, at least 6, at least 7, at least 8, at least 9 or at least 10 times. In some of these embodiments, the catalyst retains at least 80% activity (e.g., at least 90%, 95%, 96%, 97%, 98%, or 99% activity) after being recycled 1, 2, 3, 4, 5, 6, 7, 8, 9 or 10 times, when compared to the catalytic activity under identical conditions prior to being recycled.

In other embodiments, the method is performed in a continuous process, where the contents flow through the reactor with an average continuous flow rate but with no explicit mixing. After introduction of the catalyst and the feed sugar into the reactor, the contents of the reactor are continuously or periodically mixed or blended, and after a period of time, less than all of the products of the reaction are removed. In one variation, method is performed in a continuous process, where the mixture containing the catalyst and one or more sugars is not actively mixed. Additionally, mixing of catalyst and feed sugar may occur as a result of the redistribution of catalysts settling by gravity, or the non-active mixing that occurs as the material flows through a continuous reactor. In some embodiments of the methods, the steps of combining the feed sugar with a catalyst and isolating the oligosaccharide composition produced are performed concurrently.

Reactors

The reactors used for the methods described herein may be open or closed reactors suitable for use in containing the chemical reactions described herein. Suitable reactors may include, for example, a fed-batch stirred reactor, a batch stirred reactor, a continuous flow stirred reactor with ultrafiltration, a continuous plug-flow column reactor, an attrition reactor, or a reactor with intensive stirring induced by an electromagnetic field. See e.g., Fernanda de Castilhos Corazza, Flavio Faria de Moraes, Gisella Maria Zanin and Ivo Neitzel, Optimal control in fed-batch reactor for the cellobiose hydrolysis, Acta Scientiarum. Technology, 25: 33-38 (2003); Gusakov, A. V., and Sinitsyn, A. P., Kinetics of the enzymatic hydrolysis of cellulose: 1. A mathematical model for a batch reactor process, Enz. Microb. Technol., 7: 346-352 (1985); Ryu, S. K., and Lee, J. M., Bioconversion of waste cellulose by using an attrition bioreactor, Biotechnol. Bioeng. 25: 53-65(1983); Gusakov, A. V., Sinitsyn, A. P., Davydkin, I. Y., Davydkin, V. Y., Protas, O. V., Enhancement of enzymatic cellulose hydrolysis using a novel type of bioreactor with intensive stirring induced by electromagnetic field, Appl. Biochem. Biotechnol., 56: 141-153(1996). Other suitable reactor types may include, for example, fluidized bed, upflow blanket, immobilized, and extruder type reactors for hydrolysis and/or fermentation.

In certain embodiments where the method is performed as a continuous process, the reactor may include a continuous mixer, such as a screw mixer. The reactors may be generally fabricated from materials that are capable of withstanding the physical and chemical forces exerted during the processes described herein. In some embodiments, such materials used for the reactor are capable of tolerating high concentrations of strong liquid acids; however, in other embodiments, such materials may not be resistant to strong acids.

It should also be understood that additional feed sugar and/or catalyst may be added to the reactor, either at the same time or one after the other.

d) Recyclability of Catalysts

The catalysts containing acidic and ionic groups used in the methods of producing oligosaccharide compositions as described herein may be recycled. Thus, in one aspect, provided herein are methods of producing oligosaccharide compositions using recyclable catalysts.

Any method known in the art may be used to separate the catalyst for reuse, including, for example, centrifugation, filtration (e.g., vacuum filtration), and gravity settling.

The methods described herein may be performed as batch or continuous processes.

Recycling in a batch process may involve, for example, recovering the catalyst from the reaction mixture and reusing the recovered catalyst in one or more subsequent reaction cycles. Recycling in a continuous process may involve, for example, introducing additional feed sugar into the reactor, without additional of fresh catalyst.

In some of embodiments wherein at least a portion of the catalyst is recycled, the catalyst is recycled at least 1, at least 2, at least 3, at least 4, at least 5, at least 6, at least 7, at least 8, at least 9 or at least 10 times. In some of these embodiments, the catalyst retains at least 80%, at least 90%, at least 95%, at least 96%, at least 97%, at least 98%, or at least 99% activity after being recycled 1, 2, 3, 4, 5, 6, 7, 8, 9 or 10 times, when compared to the catalytic activity under identical conditions prior to being recycled.

As used herein, the “catalyst activity” refers to the effective first order kinetic rate constant for the molar conversion of reactants, k=−ln(1−X(t))/t. The molar conversion of the reactant A at time t is defined as X_{A}(t)=1−mol(A,t)/mol(A,0), where mol(A,t) refers to the number of moles of species A present in the reaction mixture at time t and mol(A,0) refers to the number of moles of species A present at the start of the reaction, t=0. In practice, the number of moles of the reactant A is often measured at several points in time, t₁, t₂, t₃, . . . , tₙ during a single reaction cycle and used to calculate the conversions X_{A}(t₁), X_{A}(t₂), . . . X_{A}(tₙ) at the corresponding times. The first order rate constant k is then calculated by fitting the data for X_{A}(t).

As used herein, a reaction “cycle” refers to one period of use within a sequence of uses of the catalyst. For example, in a batch process, a reaction cycle corresponds to the discrete steps of charging a reactor system with reactants and catalyst, heating the reaction under suitable conditions to convert the reactants, maintaining the reaction conditions for a specified residence time, separating the reaction products from the catalyst, and recovering the catalyst for re-use. In a continuous process, a cycle refers a single reactor space time during the operation of the continuous process. For example, in a 1,000 liter reactor with a continuous volumetric flow of 200 liters per hour, the continuous reactor space time is two hours, and the first two hour period of continuous operation is the first reaction cycle, the next two hour period of continuous operation is the second reaction cycle, etc.

As used herein, the “loss of activity” or “activity loss” of a catalyst is determined by the average fractional reduction in the catalyst activity between consecutive cycles. For example, if the catalyst activity in reaction cycle 1 is k(1) and the catalyst activity in reaction cycle 2 is k(2), then the loss in catalyst activity between cycle 1 and cycle 2 is calculated as [k(2)−k(1)]/k(1). Over N reaction cycles, the loss of activity is then determined as

$$\frac{1}{( N - 1 )} {\sum }_{i = 2}^{N} \frac{k ( i ) - k ( i - 1 )}{k ( i )} ,$$

measured in units of fractional loss per cycle.

In some variations, the rate constant for the conversion of additional feed sugar is less than 20% lower than the rate constant for the conversion of the reactant feed sugar in the first reaction. In certain variations, the rate constant for conversion of the additional feed sugar is less than 15%, less than 12%, less than 10%, less than 8%, less than 6%, less than 4%, less than 2%, or less than 1% lower than the rate constant for the conversion of the reactant feed sugar in the first reaction. In some variations, the loss of activity is less than 20% per cycle, less than 15% per cycle, less than 10% per cycle, less than 8% per cycle, less than 4% per cycle, less than 2% per cycle, less than 1% per cycle, less than 0.5% per cycle, or less than 0.2% per cycle.

As used herein “catalyst lifetime” refers to the average number of cycles that a catalyst particle can be re-used before it no longer effectively catalyzes the conversion of additional reactant feed sugar. The catalyst lifetime is calculated as the reciprocal of the loss of activity. For example, if the loss of activity is 1% per cycle, then the catalyst lifetime is 100 cycles. In some variations, the catalyst lifetime is at least 1 cycle, at least 2 cycles, at least 10 cycles, at least 50 cycles, at least 100 cycles, at least 200 cycles, at least 500 cycles.

In certain embodiments, a portion of the total mass of the catalyst in a reaction may be removed and replaced with fresh catalyst between reaction cycles. For example, in some variations, 0.1% of the mass of the catalyst may be replaced between reaction cycles, 1% of the mass of the catalyst may be replaced between reaction cycles, 2% of the mass of the catalyst may be replaced between reaction cycles, 5% of the mass of the catalyst may be replaced between reaction cycles, 10% of the mass of the catalyst may be replaced between reaction cycles, or 20% of the mass of the catalyst may be replaced between reaction cycles.

As used herein, the “catalyst make-up rate” refers to the fraction of the catalyst mass that is replaced with fresh catalyst between reaction cycles.

e) Additional Processing Steps

With reference again to FIG. 1, process 100 may be modified to have additional processing steps. Additional processing steps may include, for example, polishing steps. Polishing steps may include, for example, separation, dilution, concentration, filtration, demineralization, chromatographic separation, or decolorization, or any combination thereof. For example, in one embodiment process 100 is modified to include a dilution step and a decolorization step. In another embodiment process 100 is modified to include a filtration step and a drying step.

Decolorization

In some embodiments, the methods described herein further include a decolorization step. The one or more oligosaccharides produced may undergo a decolorization step using any method known in the art, including, for example, treatment with an absorbent, activated carbon, chromatography (e.g., using ion exchange resin), hydrogenation, and/or filtration (e.g., microfiltration).

In certain embodiments, the one or more oligosaccharides produced are contacted with a color-absorbing material at a particular temperature, at a particular concentration, and/or for a particular duration of time. In some embodiments, the mass of the color absorbing species contacted with the one or more oligosaccharides is less than 50% of the mass of the one or more oligosaccharides, less than 35% of the mass of the one or more oligosaccharides, less than 20% of the mass of the one or more oligosaccharides, less than 10% of the mass of the one or more oligosaccharides, less than 5% of the mass of the one or more oligosaccharides, less than 2% of the mass of the one or more oligosaccharides, or less than 1% of the mass of the one or more oligosaccharides.

In some embodiments, the one or more oligosaccharides are contacted with a color absorbing material. In certain embodiments, the one or more oligosaccharides are contacted with a color absorbing material for less than 10 hours, less than 5 hours, less than 1 hour, or less than 30 minutes. In a particular embodiment, the one or more oligosaccharides are contacted with a color absorbing material for 1 hour.

In certain embodiments, the one or more oligosaccharides are contacted with a color absorbing material at a temperature from 20 to 100 degrees Celsius, 30 to 80 degrees Celsius, 40 to 80 degrees Celsius, or 40 to 65 degrees Celsius. In a particular embodiment, the one or more oligosaccharides are contacted with a color absorbing material at a temperature of 50 degrees Celsius.

In certain embodiments, the color absorbing material is activated carbon. In one embodiment, the color absorbing material is powdered activated carbon. In other embodiments, the color absorbing material is an ion exchange resin. In one embodiment, the color absorbing material is a strong base cationic exchange resin in a chloride form. In another embodiment, the color absorbing material is cross-linked polystyrene. In yet another embodiment, the color absorbing material is cross-linked polyacrylate. In certain embodiments, the color absorbing material is Amberlite FPA91, Amberlite FPA98, Dowex 22, Dowex Marathon MSA, or Dowex Optipore SD-2.

Demineralization

In some embodiments, the one or more oligosaccharides produced are contacted with a material to remove salts, minerals, and/or other ionic species. In certain embodiments, the one or more oligosaccharides are flowed through an anionic/cationic exchange column pair. In one embodiment, the anionic exchange column contains a weak base exchange resin in a hydroxide form and the cationic exchange column contains a strong acid exchange resin in a protonated form.

Separation and Concentration

In some embodiments, the methods described herein further include isolating the one or more oligosaccharides produced. In certain variations, isolating the one or more oligosaccharides comprises separating at least a portion of the one or more oligosaccharides from at least a portion of the catalyst, using any method known in the art, including, for example, centrifugation, filtration (e.g., vacuum filtration, membrane filtration), and gravity settling. In some embodiments, isolating the one or more oligosaccharides comprises separating at least a portion of the one or more oligosaccharides from at least a portion of any unreacted sugar, using any method known in the art, including, for example, filtration (e.g., membrane filtration), chromatography (e.g., chromatographic fractionation), differential solubility, and centrifugation (e.g., differential centrifugation).

In some embodiments, the methods described herein further include a concentration step. For example, in some embodiments, the isolated oligosaccharides undergo evaporation (e.g., vacuum evaporation) to produce a concentrated oligosaccharide composition. In other embodiments, the isolated oligosaccharides undergo a spray drying step to produce an oligosaccharide powder. In certain embodiments, the isolated oligosaccharides undergo both an evaporation step and a spray drying step.

f) Bond Refactoring

Feed sugars comprising non-monomeric sugars used in the methods described herein typically have α-1,4 bonds, and when used as reactants in the methods described herein, at least a portion of the α-1,4 bonds are converted into α-1,2 bonds, β-1,2 bonds, α-1,3 bonds, β-1,3 bonds, β-1,4 bonds, α-1,6 bonds, and β-1,6 bonds, as applicable. The feed sugars may comprise non-monomeric hexoses or non-monomeric pentoses, or a combination thereof. It should be clear to one of skill in the art that α-1,6 bonds and β-1,6 bonds may not be applicable to non-monomeric pentoses.

Thus, in certain aspects, provided is a method of producing an oligosaccharide composition, by:

combining feed sugar with a catalyst to form a reaction mixture,
wherein the feed sugar has α-1,4 bonds, and
wherein the catalyst has acidic monomers and ionic monomers connected to form a polymeric backbone, or wherein the catalyst comprises a solid support, acidic moieties attached to the solid support, and ionic moieties attached to the solid support; and

converting at least a portion of the α-1,4 bonds in the feed sugar to one or more non-α-1,4 bonds selected from the group consisting of β-1,4 bonds, α-1,3 bonds, β-1,3 bonds, α-1,6 bonds, and β-1,6 bonds to produce an oligosaccharide composition from at least a portion of the reaction mixture.

It should generally be understood that α-1,4 bonds may also be referred to herein as α(1→4) bonds, and similarly, β-1,4 bonds, α-1,3 bonds, β-1,3 bonds, α-1,6 bonds, and β-1,6 bonds may be referred to as β(1→4), α(1→3), β(1→3), α(1→6), and β(1→6) bonds, respectively. It should also generally be understood that α-1,4 bonds may also be referred to herein as α-(1,4) glycyosidic linkages, and similarly, β-1,4 bonds, α-1,3 bonds, β-1,3 bonds, α-1,6 bonds, and β-1,6 bonds may be referred to as β-(1,4), α-(1,3), β-(1,3), α-(1,6), and β-(1,6) glycosidic linkages, respectively.

The following examples are meant to illustrate the invention. They are not meant to limit the invention in any way.

### Example 1

### Preparation of Catalyst

This Example demonstrates the preparation and characterization of poly-(styrene sulfonic acid-co-vinylbenzylimidazolium sulfate-co-divinylbenzene).

To a 30 L jacketed glass reactor, housed within a walk-in fume hood and equipped with a 2 inch bottom drain port and a multi-element mixer attached to an overhead air-driven stirrer, was charged 14 L of N,N-dimethylformamide (DMF, ACS Reagent Grade, Sigma-Aldrich, St. Louis, Mo., USA) and 2.1 kg of 1H-imidazole (ACS Reagent Grade, Sigma-Aldrich, St. Louis, Mo., USA) at room temperature. The DMF was stirred to dissolve the imidazole. To the reactor was then added 7.0 kg of cross-linked poly-(styrene-co-divinylbenzene-co-vinylbenzyl chloride) to form a stirred suspension. The reaction mixture was heated to 90 degrees Celsius by pumping heated bath fluid through the reactor jacket, and the reaction mixture was allowed to react for 24 hours, after which it was gradually cooled.

Then, the DMF and residual unreacted 1H-imidazole was drained from the resin, after which the retained resin was washed repeatedly with acetone to remove residual heavy solvent or unreacted reagents. The reaction yielded cross-linked poly-(styrene-co-divinylbenzene-co-1H-imidazolium chloride) as off-white spherical resin beads. The resin beads were removed from the reactor and heated at 70 degrees Celsius in air to dry.

The cleaned 30 L reactor system was charged with 2.5 L of 95% sulfuric acid (ACS Reagent Grade) and then approximately 13 L of oleum (20% free SO₃ content by weight, Puritan Products, Inc., Philadelphia, Pa., USA). To the stirred acid solution was gradually added 5.1 kg of the cross-linked poly-(styrene-co-divinylbenzene-co-1H-imidazolium chloride). After the addition, the reactor was flushed with dry nitrogen gas, the stirred suspension was heated to 90 degrees Celsius by pumping heated bath fluid through the reactor jacket, and the suspension was maintained at 90 degrees Celsius for approximately four hours. After completion of the reaction, the mixture was allowed to cool to approximately 60 degrees Celsius and the residual sulfuric acid mixture was drained from the reactor. The resin was washed with 80 wt % sulfuric acid solution, followed by 60 wt % sulfuric acid solution. Then the resin was washed repeatedly with distilled water until the pH of the wash water was above 5.0, as determined by pH paper, to yield the solid catalyst. The acid functional density of catalyst was determined to be at least 2.0 mmol H+/g dry resin by ion-exchange acid-base titration.

### Example 2

### Preparation of Oligosaccharide Compositions

This Example demonstrates the preparation oligosaccharides from different feed sugars using a catalyst with acidic and ionic moieties, prepared according to the procedure as described in Example 1 above. Various oligosaccharides were prepared at 100 g scale starting from the feed sugars listed in Table 2 below.

TABLE 2
Feed sugars used in the preparation of oligosaccharides

| # | Label | Starting Sugars | Product Oligosaccharide |
|---|---|---|---|
| 2.1 | GLOS | glucose, 100% g/g | gluco-oligosaccharide |
| | (dextrose) | | |
| 2.2 | MOS | mannose, 100% g/g | manno-oligosaccharide |
| 2.3 | GGOS (50/50) | glucose, 50% g/g | gluco-galacto- |
| | | galactose, 50% g/g | oligosaccharide |
| 2.4 | XOS | xylose, 100% g/g | xylo-oligosaccharide |
| 2.5 | GLOS (starch) | malto-dextrin, | gluco-oligosaccharide |
| | | 100% g/g | |
| 2.6 | AGOS (50/50) | arabinose, 50% g/g | arabino-galacto- |
| | | galactose, 50% g/g | oligosaccharide |
| 2.7 | XGGOS | xylose, 33.3% g/g | gluco-galacto-xylo- |
| | (33/33/33) | glucose, 33.3% g/g | oligosaccharide |
| | | galactose, 33.3% g/g | |
| 2.8 | AXOS (50/50) | arabinose, 50% g/g | arabino-xylo- |
| | | xylose, 50% g/g | oligosaccharide |
| 2.9 | GXOS (75/25) | glucose, 75% g/g | gluco-xylo- |
| | | xylose, 25% g/g | oligosaccharide |
| 2.10 | GXOS (25/75) | glucose, 25% g/g | gluco-xylo- |
| | | xylose, 75% g/g | oligosaccharide |
| 2.11 | XGGOS | glucose, 12.5% g/g | xylo-gluco-galacto- |
| | (75/12.5/12.5) | galactose, 12.5% g/g | oligosaccharide |
| | | xylose, 75% g/g | |

For each preparation, a total of 100 dry grams of feed sugars were dispensed, according to the starting mass ratios provided in Table 1, into a 400 mL glass cylindrical reactor. The mixture was gradually heated to 105° C. by heating the walls of the reactor with a temperature-controlled oil bath. Mixing was provided by an overhead mechanical stirrer equipped with a stainless steel three-blade impeller, where the ratio of the diameter of the mixing element to the diameter of the reaction vessel was approximately 0.8. During the heating process, the minimum volume of water required to bring the feed sugar mixture into a viscous syrup was dispensed. Once at temperature, catalyst was dispensed into the reactor at a total loading of 0.2 g of dry catalyst per dry gram of feed sugar. With mixing at a stir rate of approximately 100 RPM, the catalyst formed a viscous suspension, which was maintained for approximately three hours at 105° C. Over the course of the reaction, the solution thickened as oligosaccharides formed and water evaporated from the reaction vessel. The final moisture content of the reaction mixture was determined to be approximately 5%. After three hours, 100 mL of de-ionized water was dispensed into the reactor to dilute the oligosaccharide composition to approximately 50 Brix. The mixture was cooled to room temperature and the resulting oligosaccharide syrup was separated from the catalyst by vacuum filtration through a coarse membrane (pore size 50-100 micron). During filtration, additional water was used to wash residual soluble species from the catalyst, resulting in further dilution of the oligosaccharide compositions to a concentration of approximately 25% solids.

For the recovered syrup from each preparation, the syrup was decolorized using powdered activated carbon at a loading of about 1%-2% g dry activated carbon per gram solids at 65° C. for one hour, after which the decolorized syrup was recovered by vacuum microfiltration through a 0.2 micron polyether sulfone membrane. The syrup was then de-ashed by ion exchange by passing it through a column containing a food grade strong acid cationic exchange resin followed by a column containing a weak-base anionic exchange resin. The mono-saccharide and di-saccharide content of the resulting oligosaccharide syrup was removed by loading the syrup to a 10 kD dialysis tube and placing the tube into a reservoir of distilled water. The residual DP1 and DP2 content of the dialyzed oligosaccharide product was confirmed to be below 1% by HPLC.

### Example 3

### Determination of Gut Microbe Growth on Carbohydrate Food Sources

This example demonstrates the ability of various oligosaccharide compositions prepared using a catalyst with acidic and ionic moieties to manipulate selectively the growth of various common gut microbes. This example further demonstrates the inability of a wide range of common fibers (hemicellulose, pectins, and gums) to enact selective growth.

A carbohydrate library in Table 3 was prepared using the oligosaccharide compositions prepared in Example 2, several common monosaccharides, and a variety of comparative examples provided by various forms of fiber.

TABLE 3
Library of Carbohydrate Sources

| Library Entry | Carbohydrate Type | Carbohydrate Source | Label |
|---|---|---|---|
| Example 3.1 | Oligosaccharide | Example 2.1 | GLOS (dextrose) |
| Example 3.2 | Oligosaccharide | Example 2.2 | MOS |
| Example 3.3 | Oligosaccharide | Example 2.3 | GGOS (50/50) |
| Example 3.4 | Oligosaccharide | Example 2.4 | XOS |
| Example 3.5 | Oligosaccharide | Example 2.5 | GLOS (starch) |
| Example 3.6 | Oligosaccharide | Example 2.6 | AGOS (50/50) |
| Example 3.7 | Oligosaccharide | Example 2.7 | XGGOS |
| | | | (33/33/33) |
| Example 3.8 | Oligosaccharide | Example 2.8 | AXOS (50/50) |
| Example 3.9 | Oligosaccharide | Example 2.9 | GXOS (75/25) |
| Example 3.10 | Oligosaccharide | Example 2.10 | GXOS (25/75) |
| Example 3.11 | Oligosaccharide | Example 2.11 | XGGOS |
| | | | (75/12.5/12.5) |
| Comparative | C6 monomeric | glucose (purified | glucose |
| Example 3.12 | sugar | reagent) | |
| Comparative | C6 sugar amide | N-acetylglucosamine | gluNAc |
| Example 3.13 | | (purified reagent) | |
| Comparative | Synthetic | Tate & Lyle Promitor | SCF-1 |
| Example 3.14 | soluble fiber | 85 (Soluble Corn | |
| | | Fiber) | |
| Comparative | Synthetic | ADM Fibersol-2 | SCF-2 |
| Example 3.15 | soluble fiber | (Soluble Corn Fiber) | |
| Comparative | Synthetic | ADM Premidex | SWF-1 |
| Example 3.16 | soluble fiber | (Soluble Wheat Fiber) | |
| Comparative | Synthetic | LiveLong P95 | XOS |
| Example 3.17 | soluble fiber | Enzymatic xylo- | |
| | | oligosaccharide | |
| Comparative | Synthetic | Polygalacturonic | PGA |
| Example 3.18 | oectin | acid | |
| Comparative | Pectin | Pectic galactan from | PG-1 |
| Example 3.19 | | potato extract | |
| Comparative | Pectin | Rhamnogalactouronan | RG-1 |
| Example 3.20 | | from potato extract | |
| Comparative | hemicellulose | arabinan from sugar beet | ARA |
| Example 3.21 | | pulp | |
| Comparative | Pectin | Rhamnogalactouronan | RG-2 |
| Example 2.22 | | from apple peal | |
| Comparative | gum | Arabino-galactan | AG-1 |
| Example 3.23 | | from Guar gum | |

The ability of common gut microbes to grow on carbohydrate food sources was measured using a custom carbohydrate array constructed in a 96-well format. See for example, Martens E C, et al, “Recognition and Degradation of Plant Cell Wall Polysaccharides by Two Human Gut Symbionts,” PLOS Biology, volume 9, issue 12, page e1001221 (2011) and Martens E C, Chiang H C, Gordon J I, “Mucosal glycan foraging enhances fitness and transmission of a saccharolytic human gut bacterial symbiont.” Cell Host Microbe 4: 447-457 (2008). Each well of a flat bottom 96-well plate was loaded with 100 μL of each sterilized carbohydrate stock at 2× concentration.

Each substrate was represented in triplicate on each assay plate in two non-adjacent wells. Two carbohydrate-free water wells were included as negative controls. Cultures for assay inoculations of Bacteroides eggerthii, Bacteroides intestinalis, Bacteroides vulgatus, Bacteroides caccae, Bacteroides finegoldii, Bacteroides salyersiae, Bacteroides thetaiotamicron, Bacteroides uniformis, Bacteroides cellulosilyticus, Bacteroides dorei, Bacteroides clarus, Bacteroides fragillis, Bacteroides Bacteroides ovatus, Bacteroides oleiciplenus, Bacteroides xylanisolvens, Dysgonomonas gadei, Dysgonomonas mossii Parabacteroides johnsonii, Parabacteroides goldsteinii, Parabacteroides merdae, Parabacteroides distasonis, Parabacteroides gordonii, and Odoribacter splanchnicus were grown for 24 h at 37° C. under an atmosphere of 10% H2, 5% CO2, and 85% N2 in suitable glucose-containing media. 1 mL aliquots, centrifuged to pellet bacteria, were then gently re-suspended in carbohydrate-free media and used to inoculate 50 mL of carbohydrate-free media at a ratio of 1:50. Each carbohydrate array was loaded with 100 μL of the inoculated medium to produce 96 individual 200 μL cultures. Assay plates were sealed in an anaerobic chamber under the atmosphere noted above with an optically clear gas-permeable polyurethane membrane. Plates were then loaded into an automated plate handling device coupled to an absorbance reader. Absorbance at 600 nm (A600) was measured for each well at 10-15 min intervals.

Absorption data versus time were averaged across replicate samples to produce a growth curve for each carbohydrate-organism pairing. For a given pairing, the organism was considered to be capable of growing on the food source if a measurable increase in absorption was observed in the growth curve (at least approximately 10% of the absorption measured for the same organism grown on glucose).

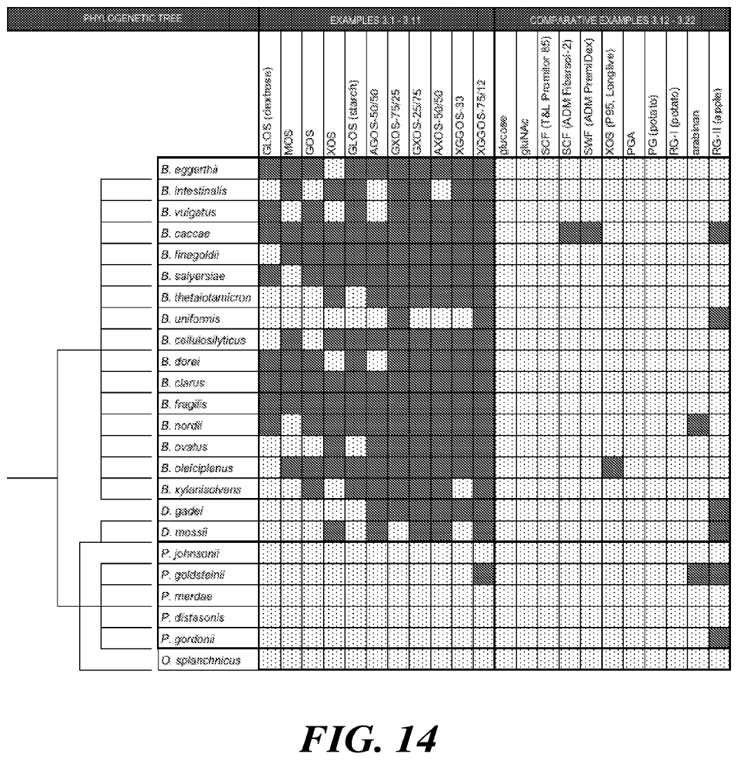

FIG. 14 provides a summary of the resulting growth data with the various gut microbes represented by rows and the various carbohydrate food sources represented by columns. Dark grey cells indicate organism-carbohydrate pairings in which growth did not occur and light grey-hashed cells indicate organism-carbohydrate pairings where growth was observed.

As expected, all of the gut microflora cultures grew well on both glucose and N-acetylglucosamine. Additionally, the vast majority of microbial species grew well on soluble fiber, pectic galactans, pectins, and hemicellulose, with no apparent selectivity.

Surprisingly, the oligosaccharide compositions exhibited selective growth among organisms with a common genus. Oligosaccharide 3.1 was consumed by Odoribacter, Parabacteroides, and Dysgonomonas, but only 50% of the Bacteroides species tested. Oligosaccharide 3.5 was similarly consumed by Odoribater, Parabacteroides and Dysgonomonas, but only 20% of the Bacteroides species tested. Conversely, Oligosaccharides 3.6, 3.8, 3.9 and 3.11 are not consumed by the Dysgonomonas species tested.

### ENUMERATED EMBODIMENTS

The present disclosure also includes the following enumerated embodiments.

### Embodiment 1

A method of enhancing growth in an animal, comprising:

providing a composition to the animal; and

enhancing growth in the animal, wherein the composition comprises:
(a) at least one C5 carbohydrate, at least one C6 carbohydrate, at least one C5 deoxy sugar, at least one C6 deoxy sugar, at least one C5 amino sugar, at least one C6 amino sugar, at least one C5 sugar alcohol, at least one C6 sugar alcohol, at least one C5 sugar acid, at least one C6 sugar acid, at least one C5 phosphate sugar, at least one C6 phosphate sugar, at least one C5 sulfate sugar, or at least one C6 sulfate sugar, or
a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or
any combinations thereof; and
(b) a delivery vehicle.

### Embodiment 2

The method of embodiment 1, wherein the growth of the animal is enhanced relative to an animal that is not administered the composition.

### Embodiment 3

A method of decreasing feed conversion ratio of feed provided to an animal, comprising:
providing feed to the animal; and
decreasing the feed conversion ratio (FCR) of feed provided to the animal,
wherein the feed comprises:
(a) a base feed;
(b) at least one C5 carbohydrate, at least one C6 carbohydrate, at least one C5 deoxy sugar, at least one C6 deoxy sugar, at least one C5 amino sugar, at least one C6 amino sugar, at least one C5 sugar alcohol, at least one C6 sugar alcohol, at least one C5 sugar acid, at least one C6 sugar acid, at least one C5 phosphate sugar, at least one C6 phosphate sugar, at least one C5 sulfate sugar, or at least one C6 sulfate sugar, or
a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or
any combinations thereof; and
(c) a delivery vehicle.

### Embodiment 4

The method of embodiment 3, wherein the feed conversion ratio (FCR) is between 0 to 4% higher than the performance target minimum

### Embodiment 5

The method of embodiment 3 or 4, wherein the feed conversion ratio is decreased by between 0 to 4%.

### Embodiment 6

A method of treating a disease or disorder in an animal in need thereof, comprising administering a composition to the animal,
wherein the composition comprises:
(a) at least one C5 carbohydrate, at least one C6 carbohydrate, at least one C5 deoxy sugar, at least one C6 deoxy sugar, at least one C5 amino sugar, at least one C6 amino sugar, at least one C5 sugar alcohol, at least one C6 sugar alcohol, at least one C5 sugar acid, at least one C6 sugar acid, at least one C5 phosphate sugar, at least one C6 phosphate sugar, at least one C5 sulfate sugar, or at least one C6 sulfate sugar, or
a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or
any combinations thereof; and
(b) a delivery vehicle.

### Embodiment 7

The method of embodiment 6, wherein the disease or disorder is necrotic enteritis, coccidiosis, nutrient malabsorption syndrome, intestinal barrier breakdown, colisepticemia, yolk sack infection, salmonella infection, or campylobacter infection.

### Embodiment 8

The method of embodiment 6 or 7, wherein the disease or disorder has a lower incidence in the animal compared to an animal that is not administered the composition.

### Embodiment 9

A method of modulating the gut microbiome of an animal, comprising:

administering a composition to the animal; and

modulating the gut microbiome of the animal,

wherein the therapeutic composition comprises a carbohydrate composition and a pharmaceutically acceptable excipient,

wherein the composition comprises:
(a) at least one C5 carbohydrate, at least one C6 carbohydrate, at least one C5 deoxy sugar, at least one C6 deoxy sugar, at least one C5 amino sugar, at least one C6 amino sugar, at least one C5 sugar alcohol, at least one C6 sugar alcohol, at least one C5 sugar acid, at least one C6 sugar acid, at least one C5 phosphate sugar, at least one C6 phosphate sugar, at least one C5 sulfate sugar, or at least one C6 sulfate sugar, or
a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or
any combinations thereof; and
(b) a delivery vehicle.

### Embodiment 10

A method of targeting a region of the gastrointestinal tract in an animal, comprising:

administering a composition to the animal; and

targeting a region of the gastrointestinal tract in the animal for modulation of gut microbiota,

wherein the composition comprises:
(a) at least one C5 carbohydrate, at least one C6 carbohydrate, at least one C5 deoxy sugar, at least one C6 deoxy sugar, at least one C5 amino sugar, at least one C6 amino sugar, at least one C5 sugar alcohol, at least one C6 sugar alcohol, at least one C5 sugar acid, at least one C6 sugar acid, at least one C5 phosphate sugar, at least one C6 phosphate sugar, at least one C5 sulfate sugar, or at least one C6 sulfate sugar, or
a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or
any combinations thereof; and
(b) a delivery vehicle.

### Embodiment 11

The method of embodiment 10, wherein the region of the gastrointestinal tract in the animal is ileum, cecum, or a combination thereof.

### Embodiment 12

The method of any one of embodiments 1 to 11, wherein the composition further comprises at least one pharmaceutically acceptable vehicle.

### Embodiment 13

The composition of any one of embodiments 1 to 12, wherein the composition is an aqueous solution, a liquid concentrate, a colloidal suspension, a syrup, a tablet, a capsule, a pill, a lozenge, a cream, a gel, a foam, a powder, or granulated.

### Embodiment 14

The composition of any one of embodiments 1 to 13, wherein the composition is administered orally to the animal.

### Embodiment 15

The method of any one of embodiments 1 to 14, wherein the composition is administered to the animal in an animal feed composition.

### Embodiment 16

The method of any one of embodiments 1 to 15, wherein the animal is other than a human.

### Embodiment 17

The method of any one of embodiments 1 to 16, wherein the animal is selected from the group consisting of poultry or swine.

### Embodiment 18

The method of embodiment 17, wherein the poultry is selected from the group consisting of chickens, geese, ducks, turkeys, quail, and Cornish game hens.

### Embodiment 19

An animal feed composition, comprising:
(a) a base feed;
(b) at least one C5 carbohydrate, at least one C6 carbohydrate, at least one C5 deoxy sugar, at least one C6 deoxy sugar, at least one C5 amino sugar, at least one C6 amino sugar, at least one C5 sugar alcohol, at least one C6 sugar alcohol, at least one C5 sugar acid, at least one C6 sugar acid, at least one C5 phosphate sugar, at least one C6 phosphate sugar, at least one C5 sulfate sugar, or at least one C6 sulfate sugar, or
   a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or
   any combinations thereof; and
(c) a delivery vehicle.

### Embodiment 20

An animal feed pre-mix, comprising:
(a) a carrier material;
(b) at least one C5 carbohydrate, at least one C6 carbohydrate, at least one C5 deoxy sugar, at least one C6 deoxy sugar, at least one C5 amino sugar, at least one C6 amino sugar, at least one C5 sugar alcohol, at least one C6 sugar alcohol, at least one C5 sugar acid, at least one C6 sugar acid, at least one C5 phosphate sugar, at least one C6 phosphate sugar, at least one C5 sulfate sugar, or at least one C6 sulfate sugar, or
   a compound comprising 2 to 5 units, wherein each unit is independently a C5 carbohydrate unit, a C6 carbohydrate unit, a C5 deoxy sugar unit, a C6 deoxy sugar unit, a C5 amino sugar unit, a C6 amino sugar unit, a C5 sugar alcohol unit, a C6 sugar alcohol unit, a C5 sugar acid unit, a C6 sugar acid unit, a C5 phosphate sugar unit, a C6 phosphate sugar unit, a C5 sulfate sugar unit, or a C6 sulfate sugar unit, or
   any combinations thereof; and
(c) a delivery vehicle.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. The disclosures of U.S. Provisional Application No. 62/255,348, filed Nov. 13, 2015 and U.S. Provisional Application No. 62/255,352, filed Nov. 13, 2015 are hereby incorporated by reference in their entirety.

Other embodiments are in the claims.

What is claimed is:

1. A method of treating a disease or disorder in an animal in need thereof, comprising administering a therapeutic composition to the animal, wherein the therapeutic composition comprises an oligosaccharide composition, and optionally at least one pharmaceutically acceptable vehicle,
wherein the oligosaccharide composition has a glycosidic bond type distribution of:
at least 10 mol % α-(1,3) glycosidic linkages; and
at least 10 mol % β-(1,3) glycosidic linkages, and
wherein at least 10 dry wt % of the oligosaccharide composition has a degree of polymerization of at least 3.

2. The method of claim 1, wherein the disease or disorder is necrotic enteritis, coccidiosis, nutrient malabsorption syndrome, intestinal barrier breakdown, colisepticemia, yolk sack infection, salmonella infection, or campylobacter infection.

3. The method of claim 1, wherein the disease or disorder has a lower incidence in the animal compared to an animal that is not administered the therapeutic composition.

4. A method of modulating the gut microbiome of an animal, comprising:
administering a therapeutic composition to the animal; and
modulating the gut microbiome of the animal,
wherein the therapeutic composition comprises an oligosaccharide composition, and optionally at least one pharmaceutically acceptable vehicle,
wherein the oligosaccharide composition has a glycosidic bond type distribution of:
at least 10 mol % α-(1,3) glycosidic linkages; and
at least 10 mol % β-(1,3) glycosidic linkages, and
wherein at least 10 dry wt % of the oligosaccharide composition has a degree of polymerization of at least 3.

5. A method of targeting a region of the gastrointestinal tract in an animal, comprising:
administering a therapeutic composition to the animal; and
targeting a region of the gastrointestinal tract in the animal for modulation of gut microbiota,
wherein the therapeutic composition comprises an oligosaccharide composition, and optionally at least one pharmaceutically acceptable vehicle,
wherein the oligosaccharide composition has a glycosidic bond type distribution of:
at least 10 mol % α-(1,3) glycosidic linkages; and
at least 10 mol % β-(1,3) glycosidic linkages, and
wherein at least 10 dry wt % of the oligosaccharide composition has a degree of polymerization of at least 3.

6. The method of claim 5, wherein the region of the gastrointestinal tract in the animal is ileum, cecum, or a combination thereof.

7. The method of claim 1, wherein the oligosaccharide composition has a glycosidic bond type distribution of less than 9 mol % α-(1,4) glycosidic linkages, and less than 19 mol % α-(1,6) glycosidic linkages.

8. The method of claim 1, wherein the oligosaccharide composition has a glycosidic bond type distribution of at least 15 mol % β-(1,2) glycosidic linkages.

9. The method of claim 1, wherein the oligosaccharide composition comprises an oligosaccharide selected from the group consisting of a gluco-oligosaccharide, a galacto-oligosaccharide, a fructo-oligosaccharide, a manno-oligosaccharide, a gluco-galacto-oligosaccharide, a gluco-fructo-oligosaccharide, a gluco-manno-oligosaccharide, a gluco-arabino-oligosaccharide, a gluco-xylo-oligosaccharide, a galacto-fructo-oligosaccharide, a galacto-manno-oligosaccharide, a galacto-arabino-oligosaccharide, a galacto-xylo-oligosaccharide, a fructo-manno-oligosaccharide, a fructo-arabino-oligosaccharide, a fructo-xylo-oligosaccharide, a manno-arabino-oligosaccharide, and a manno-xylo-oligosaccharide, or any combinations thereof.

10. The method of claim 1, wherein the oligosaccharide composition comprises an oligosaccharide selected from the group consisting of an arabino-oligosaccharide, a xylo-oligosaccharide, and an arabino-xylo-oligosaccharide, or any combinations thereof.

11. The method of claim 1, wherein the oligosaccharide composition has a glycosidic bond type distribution of:
between 0 to 20 mol % α-(1,2) glycosidic linkages;
between 0 to 45 mol % β-(1,2) glycosidic linkages;
between 1 to 30 mol % α-(1,3) glycosidic linkages;
between 1 to 20 mol % β-(1,3) glycosidic linkages;
between 0 to 55 mol % β-(1,4) glycosidic linkages; and
between 10 to 55 mol % β-(1,6) glycosidic linkages.

12. The method of claim 1, wherein at least 50 dry wt % of the oligosaccharide composition has a degree of polymerization of at least 3.

13. The method of claim 1, wherein between 65 to 80 dry wt % of the oligosaccharide composition has a degree of polymerization of at least 3.

14. The method of claim 1, wherein at least 50 dry wt % of the oligosaccharide composition comprises one or more gluco-oligosaccharides.

15. The method of claim 1, wherein at least 50 dry wt % of the oligosaccharide composition comprises one or more gluco-galacto-oligosaccharides.

16. The method of claim 1, wherein the oligosaccharide composition has a glycosidic bond type distribution of:
between 0 to 20 mol % α-(1,2) glycosidic linkages;
between 10 to 45 mol % β-(1,2) glycosidic linkages;
between 1 to 30 mol % α-(1,3) glycosidic linkages;
between 1 to 20 mol % β-(1,3) glycosidic linkages;
between 0 to 55 mol % β-(1,4) glycosidic linkages;
between 10 to 55 mol % β-(1,6) glycosidic linkages;
less than 9 mol % α-(1,4) glycosidic linkages; and
less than 19 mol % α-(1,6) glycosidic linkages.

17. The method of claim 1, wherein the oligosaccharide composition has a glycosidic bond type distribution of:
between 0 to 15 mol % α-(1,2) glycosidic linkages;
between 0 to 15 mol % β-(1,2) glycosidic linkages;
between 1 to 20 mol % α-(1,3) glycosidic linkages;
between 1 to 15 mol % β-(1,3) glycosidic linkages;
between 5 to 55 mol % β-(1,4) glycosidic linkages;
between 15 to 55 mol % β-(1,6) glycosidic linkages;
less than 20 mol % α-(1,4) glycosidic linkages; and
less than 30 mol % α-(1,6) glycosidic linkages.

18. The method of claim 1, wherein the oligosaccharide composition is a functionalized oligosaccharide composition.

19. The method of claim 1, wherein the therapeutic composition comprises at least one pharmaceutically acceptable vehicle.

20. The method of claim 1, wherein the therapeutic composition is an aqueous solution, a liquid concentrate, a colloidal suspension, a syrup, a tablet, a capsule, a pill, a lozenge, a cream, a gel, a foam, a powder, or granulated.

21. The method of claim 1, wherein the therapeutic composition is administered orally to the animal.

22. The method of claim 1, wherein the therapeutic composition is administered to the animal in an animal feed composition.

23. The method of claim 1, wherein the animal is other than a human.

24. The method of claim 1, wherein the animal is selected from the group consisting of poultry or swine.

25. The method of claim 24, wherein the poultry is selected from the group consisting of chickens, geese, ducks, turkeys, quail, and Cornish game hens.

26. An animal feed composition, comprising:
(a) a base feed;
(b) an oligosaccharide composition having a glycosidic bond type distribution of:
at least 10 mol % α-(1,3) glycosidic linkages; and
at least 10 mol % β-(1,3) glycosidic linkages, and
at least 10 dry wt % of the oligosaccharide composition has a degree of polymerization of at least 3; and
(c) at least one pharmaceutically acceptable vehicle.
